(19) United States
(12) Patent Application Publication
(10) Pub. No.: US 2003/0161894 A1
(43) Pub. Date: Aug. 28, 2003

(54) Precipitated aragonite and a process for producing it

(75) Inventor: Isaac Yaniv, Nesher (IL)

Correspondence Address: BROWDY AND NEIMARK, P.L.L.C., 624 NINTH STREET, NW, SUITE 300, WASHINGTON, DC, 20001-5303, US

(73) Assignee: 3P TECHNOLOGIES LTD., Nesher (IL)

(21) Appl. No.: 09/946,139

(22) Filed: Sep. 5, 2001

(51) Int. Cl. A61K 33/10; C01B 31/24; A61K 31/202

(52) U.S. Cl. 424/687; 514/560; 423/419.1

(57) ABSTRACT

Disclosed is a novel form of particulate precipitated aragonite, and a novel process for producing it.

### FIELD AND BACKGROUND OF THE INVENTION

The invention relates to a novel form of particulate precipitated calcium carbonate, and particularly to a novel form of particulate precipitated aragonite, and to a novel process for producing it.

Various routes are known for the production of calcium carbonate, which finds use as a thickening material, as a filler, as an extender, and most of all as a pigment, in a variety of industries such as pharmaceuticals, agrochemicals, plastics, adhesives, printing, coating (paint), paper, rubber and in filtration. For such purposes, there may be used ground calcium carbonate (GCC) or precipitated calcium carbonate (PCC). PCC in general possesses advantages over GCC, in that it is economical to produce and its precise composition, or purity, can be more strictly controlled.

The most frequently used chemical process for producing PCC is based on the carbonation of aqueous suspensions of calcium hydroxide (also known as “milk of lime” or “slaked lime”) with carbon dioxide gas, or with a carbon dioxide containing gas. This process gives rise to relatively pure precipitated calcium carbonate and is a preferred process, because there are no serious problems of contamination of the product with undesired salts, and moreover it can be controlled in order to adjust the properties of the final product. Thus, the process is based essentially on four stages: firstly, calcination of raw limestone to produce calcium oxide or “quicklime” and carbon dioxide gas or a carbon dioxide containing gas; secondly, “slaking” of the quicklime with water to produce an aqueous suspension of calcium hydroxide; thirdly, carbonation of the calcium hydroxide with carbon dioxide gas or a carbon dioxide containing gas; and finally, downstream operations such as dewatering, drying, deagglomeration, grinding, surface treatment, surface coating, mixing with other minerals (e.g. titanium dioxide, talc, kaolin, GCC, PCC—including aragonite PCC) and dyeing, which allow optimization of the properties of the precipitated calcium carbonate particles in order to be adapted to their intended uses.

Calcium carbonate can be precipitated from aqueous calcium hydroxide slurries or solutions in three different crystallographic forms (polymorphs): the vaterite form which is thermodynamically unstable, the aragonite form which is metastable under normal ambient conditions of temperature and pressure, and the calcite form which is the most stable and the most abundant in nature. These forms of calcium carbonate can be prepared by carbonation of slaked lime by suitable variations of the process conditions.

The calcite form is easy to produce on industrial scales, as precipitated calcium carbonate particles. It exists in several different shapes, of which the most common are the rhombohedral shape and the scalenohedral shape.

Aragonite forms crystals having a length/width ratio (hereinafter—“aspect ratio”) in the range between >1:1 and 100:1 of which a typical aspect ratio is 10, in which case the aragonite forms long, thin needles. Therefore, aragonite having a high aspect ratio may be denoted hereinafter—“acicular aragonite” or “needle-shaped aragonite”.

PCC particles are used as thickening materials, fillers, extenders and, most of all, as inexpensive pigments. The latter use implies that a particularly desirable property of this material is its light scattering characteristics, in order to be able to impart opacity to the products containing it. Such characteristics are optimized, when the pigment particles are very effectively dispersed and are apart by an average distance in the range between 0.2 μm and 0.4 μm in their final products, and their size distribution is in the range between 0.2 μm and 0.4 μm, namely, in the range of half a wavelength of the visible light. That means that either the production of the PCC should be adjusted to produce small particles in order to avoid expensive downstream particle size reduction operations and to cope with the expensive problems of dewatering and drying the product, or, alternatively, the process should be adjusted to produce large particles, and subsequently effect the downstream dewatering and grinding operations. In both cases, the production costs of precipitated calcium carbonate of pigment grades may be doubled or tripled just because of these unavoidable downstream steps.

High light scattering pigments currently available to the above-mentioned industries include titanium dioxide (TiO₂) particles, which are very effective to scatter the light due to their relatively high refractive index (2.76; for the rutile form) and their meticulously controlled particle size distribution of which median is in the range between 0.2 μm and 0.4 μm. However, this product is of a high specific gravity (˜4.0 g/cm³), of a high surface area due to its small particles, and most of all, is quite expensive. Fine kaolin particles are also being used as pigments, but this product, which has a much lower refractive index (1.56), is of limited opacity and is still relatively expensive. Particulate calcium carbonate could be the ideal least expensive pigment for replacing much more of the titanium dioxide and kaolin pigments in their respective present applications, if it would have improved light scattering properties.

Calcium carbonate pigments are produced in part by grinding coarse natural rocks and in part by precipitation processes. Of the precipitated calcium carbonate particles, the particulate precipitated aragonite is considered to be the most effective light scattering calcium carbonate pigment, and depending on the crystallographic surfaces its refractive indices are 1.530, 1.681 and 1.685, with a specific gravity that is substantially above 2.5 g/cm³. However, such refractive indices are too low to compete with the TiO₂ pigments (naturally, this is also true with respect to all the other forms of CaCO₃).

The basic equations that allow to compare most objectively (through hiding power, contrast ratio, and opacity measurements) the abilility of pigments to opacify the products in which they are included, show that in most of the practical cases (e.g. in coatings, paper and plastics) the refractive index of the respective pigment is the single most important factor, and therefore, all forms of CaCO₃ are by far inferior in their optical properties to TiO₂.

The light scattering effect of a pigment can be improved by trapping bubbles around or within the pigment particles. This phenomenon has been exploited very successfully, at least, by Rolim & Haas company in their organic polymeric pigment—Ropaque®, as it will be described in more detals hereinafter, but it has not been reported or exploited in the case of PCC and no such PCC pigment exists in the market today, yet.

While the majority of references, cited hereinafter, relate to the technology for producing a particulate precipitated aragonite, some of the references are included in order to better present the state of the art for the production of PCC more generally, including the downstream operations, which may be common to prior processes and may also be applicable as a downstream step that can follow the process of the invention.

### PRIOR ART

1. U.S. Pat. No. 2,081,112 (N. Statham et al.) describes a process for producing precipitated calcium carbonate by carbonating milk of lime with carbon dioxide containing gas, where the temperature in the gas absorber is maintained at 50-60° C., preferably around 55° C. It is recognized that the more violent the agitation in the gas absorber, the finer will be the product; the aim being to create a fine mist of calcium hydroxide slury.

2. U.S. Pat. No. 2,964,382 (G. E. Hall, Jr.) describes production of precipitated calcium carbonate by various chemical routes, in which calcium ions are contacted with carbonate ions in a precipitation zone, the process including also carbonation of milk of lime with carbon dioxide gas. A high shear stator/rotor agitator is used to provide turbulence by rotating at a peripheral speed of at least 1160 feet per minute (589 cm per second) in the precipitation zone. Also, this patent teaches that it is desirable to operate the process at pH values of at least 8.5 and that at temperatures above 60° C., needle-shaped precipitated aragonite particles are formed, which however produce an adverse flow property effect.

3. U.S. Pat. No. 3,320,026 (W. F. Waldeck) describes the production of various forms of precipitated calcium carbonate.

4. GB Patent No. 941,900 (assigned to Kaiser Aluminium & Chemical corporation) describes the production of precipitated aragonite particles, for use as a filter aid, by reacting continuously sodium carbonate solution and aqueous calcium hydroxide slurry at temperatures higher than 60° C. in a multistage system. The product and the solution are withdrawn at the third stage from the bottom of the reactor, the product is then separated from the solution and part of the crystals are recycled to the various stages of the process as seeds for farther precipitation of the precipitated aragonite particles.

5. CA Patent No. 765756 (J. Maskal et al.) describe the production of mixtures of aragonite and calcite PCC that contain from 15 to 60 weight percent of aragonite. The process is preferably conducted in a batchwise mode using Ca⁺⁺ solutions that contain CaCO₃ “seeds” (which were produced previously) and Ca(OH)₂/Mg(OH)₂ in molar ratios of between 0.5 and 2.0.

6. U.S. Pat. No. 3,669,620 (M. C. Bennett et al.) describes a continuous process for the production of a particulate precipitated aragonite by carbonating aqueous calcium hydroxide slurry in sucrose solutions. However, due to the cost of the sucrose, the solution had to be recycled and detrimental materials had to be removed by anion exchange resin. The preferred temperature range was between 60° C. and 90° C.; the pH values were in the range between 7 and 9; and the concentration of the calcium hydroxide was quite low—in the range between one-half and one-twentieth molar.

7. U.S. Pat. No. 4,018,877 (R. D. A. Woode) describes carbonation of calcium hydroxide slurry, wherein a complexing agent for heavy metals is added to the suspension in the gas absorber, after the calcium carbonate primary nucleation stage and before completion of the carbonation step, the complexing agent being carboxylic acids such as citric acid, ethylenediamine tetraacetic acid (EDTA), aminotriacetic acid, aminodiacetic acid or a hydroxy polycarboxylic acid. Optionally, long-chain fatty acids or their salts can be added, preferably, after the final carbonation stage.

8. U.S. Pat. No. 4,157,379 (J. Arika et al.) describes the production of a chain-structured precipitated calcium carbonate by the carbonation of calcium hydroxide suspended in water in the presence of chelating agents, such as aliphatic carboxylic acids, and water-soluble metal salts.

9. U.S. Pat. No. 4,244,933 (H. Shibazaki et al.) describes a multi-stage production process for producing a particulate precipitated aragonite, using aqueous calcium hydroxide slurry and carbon dioxide gas or a carbon dioxide containing gas, in the presence of phosphoric acids and water-soluble salts thereof.

10. U.S. Pat. No. 4,420,341 (T. H. Ferrigno) describes inorganic fillers (including calcium carbonate) surface modified with carboxylic acids, antioxidants and high-boiling non-reactive liquid agents.

11. GB Patent No. 2,145,074 (T. Shiraishi et al.) describe the process for producing the aragonite PCC. The specific gravity of the product was determined in this patent to be 2.75-2.93 g/cm³, which is a well known value for aragonite. However, no connection was made, in any way, between the measured specific gravity of the aragonite and its quality as a pigment. The carboxylic acids that are being used therein are usually being exploited to produce PCC with less heavy metal contaminants, and which have been mentioned quite often in the literature.

12. JP Patent Publication No. 63260815 (H. Shibata et al.) describes the production of a particulate precipitated aragonite, by reacting carbon dioxide gas with an aqueous calcium hydroxide slurry in presence of phosphoric acid, a phosphoric acid compound, a barium compound and a strontium compound.

13. JP Patent No. 1261225 (H. Shibata et al.) describes reacting carbon dioxide gas with an aqueous calcium hydroxide slurry, in order to produce a particulate precipitated aragonite, which is stated to have improved properties compared with particulate precipitated calcite.

14. U.S. Pat. No. 4,824,654 (Y. Ota et al.) describes a process for producing precipitated needle-shaped (5-100 μm) particulate precipitated aragonite, in which a relatively dilute aqueous calcium hydroxide solution (0.04-0.17 wt. %) and carbon dioxide gas or a carbon dioxide-containing gas are reacted together at a temperature of not less than 60° C., in a continuous or semi-continuous (intermittent) manner.

15. U.S. Pat. No. 5,043,017 (J. D. Passaratti) describes a process for producing acid-stabilized precipitated calcium carbonate particles.

16. U.S. Pat. No. 5,164,172 (H. Katayama et al.) describes a process for producing a particulate precipitated aragonite, in which a mixture of aqueous calcium hydroxide slurry, aragonite calcium carbonate particles and a water-soluble phosphoric acid compound are premixed prior to the addition of carbon dioxide gas.

17. U.S. Pat. No. 5,342,600 (I. S. Bleakley et al.) describes a process of producing particulate precipitated calcium carbonate, in which aqueous calcium hydroxide slurries of varying concentrations are reacted with carbon dioxide-containing gas under a controlled mixing speed. It is recommended therein to prepare the aqueous calcium hydroxide suspension under high shear mixing and subsequently to lower the energy and shear agitation in the reaction mixture in which the precipitated calcium carbonate particles are formed.

18. U.S. Pat. No. 5,376,343 (P. M. Fouche) describes a process for producing various forms PCC using clear solutions of Ca⁺⁺ ions. In the case of aragonite, a mixture of very dilute aqueous calcium hydroxide solution (<1%) and a water-soluble source of specific anions (e.g. ammonium nitrate) are premixed prior to addition of CO₂ gas. In this patent it is recommended to introduce fatty acids into the carbonation reactor as “an anti-caking flocculation aiding agent” for the PCC (Calcite; Aragonite and Vaterite).

19. U.S. Pat. No. 5,380,361 (R. A. Gill) describes inter alia calcium carbonate particles coated with C₁₂-C₂₂ fatty acid salts.

20. U.S. Pat. No. 5,593,489 (K -T. Wu) describes a process for producing acid-resistant calcium carbonate particles for making neutral to weakly acid paper.

21. U.S. Pat. No. 5,833,747 (I. S. Bleakley et al.) describes a process for producing a particulate precipitated aragonite, in which an aqueous calcium hydroxide slurry (148 g Ca(OH)₂ per liter of suspension) is reacted with carbon dioxide gas at an exceptionally slow rate of 0.0026 moles per minute per mole of Ca(OH)₂ in a batch operation.

22. WO 9852870 (B. Jackson et al.) describes a multi-stage commercial process for producing a particulate precipitated aragonite, using coarse-grained precipitated aragonite particles as a seeding material. Though the process is claimed to be industrially applicable, it is quite slow and thus of very limited economical value.

23. U.S. Pat. No. 5,846,500 (J. W. Bunger et al.) describes a process for producing a particulate precipitated aragonite, in which an aqueous calcium hydroxide solution is reacted with CO₂ gas in a plug-flow reaction system.

24. U.S. Pat. No. 5,846,382 (A. von Raven) describes a process for producing inorganic fillers and pigments, including particulate calcium carbonate, of improved whiteness, brightness and chromaticity.

25. U.S. Pat. No. 5,861,209 (W. J. Haskins et al.) describes a process for producing a particulate precipitated aragonite, for printing, in which an aqueous calcium hydroxide slurry is first mixed with precipitated aragonite particles for seeding and then it is reacted quite slowly with carbon dioxide gas in a batch operation. After dewatering the product to a cake containing about 70% solids, it is mixed with a typical dispersant, e.g. sodium polyacrylate, and it is further dispersed. This patent discloses the use of mixtures of a particulate precipitated aragonite, with TiO₂ and other inorganic fillers, pigments and flame retardants.

26. U.S. Pat. No. 5,939,036 (A. L. Porter et al.) describes a process for producing a particulate precipitated aragonite, in which aqueous mixtures of organic compounds and acids (e.g. ethanolamine and HCl) are used to dissolve impure CaO and to form a calcium hydroxide mixture, which is then reacted with carbon dioxide gas to yield various forms of PCC, depending on the temperature. Controlling the temperature of the carbonation at about 95° C. leads to aragonite.

27. U.S. Pat. No. 6,022,517 and U.S. Pat. No. 6,071,336 (G. H. Fairchild et al.; both assigned to Minerals Technologies, Inc.) describe a process for producing mixtures of precipitated acicular calcite and acicular aragonite particles in the ratio of 75:25 to 25:75, by reacting carbon dioxide gas or a carbon dioxide containing gas and aqueous calcium hydroxide in the presence of a water soluble aluminum compound, by controlling the specific conductivity in a range >4.0 and up to about 7.0, milliSiemens/cm, at a reaction temperature of from 25-60° C.

28. U.S. Pat. No. 6,156,286 (S. Fortier et al.) describes a process for preparing aragonite PCC by seeding the carbonation reaction with aragonite crystals, which are formed by interrupting the CO₂ feed, intermittently.

In addition:

29. “TiO₂ versus alternative white minerals”, Industrial Minerals, May 2001 (A. Cole, Assistant Editor), gives an overview of the present state of the art of industrial white minerals. According to this recent paper there is no white mineral that challenges yet the TiO₂ pigments, even though the latter are quite expensive.

30. Pigment Handbook (Vol. I-III; Edited by T. C. Patton; John Wiley & Sons, New York (1973)) describes the properties, the production processes and various uses of aragonite calcium carbonate pigment (c.f. Vol. I; Pages 119-128), as well as those of other pigments that compete in the same market like titanium dioxide, kaolin, GCC, etc. The discussion concerning the influence of the film porosity (the percentage of air in the space surrounding the pigment particles) on the hiding power (H.P.) or opacity of a coating film (c. f. Vol. III; Pages 203-217 and especially on Page 212) may help in understanding some of the aspects associated with the present invention.

31. U.S. Pat. No. 4,427,836 (A. Kowalski et al.), U.S. Pat. No. 4,469,825 (A. Kowalski et al.), and U.S. Pat. No. 4,985,064 (G. H. Redlich et al.), all assigned to Rohm & Haas, disclose an organic polymeric pigment that is produced in such ways that allow the formation of “cores” or “voids” or “microvoids” within the polymeric particles, in which water is introduced deliberately. After mixing this pigment in paint formulations or in paper and drying them, the water in the “cores” are replaced by trapped air. This, in turn, leads to a dramatic enhancement of the hiding power of paint or paper products that contain Ropaque®.

### SUMMARY OF THE INVENTION

In accordance with the present invention, a novel composition of matter is provided comprising particulate precipitated aragonite calcium carbonate having a specific gravity below about 2.5 g/cm³. Particulate precipitate aragonite calcium carbonate with these properties is characterized by its high hiding power (a result of high effective refractive index), low bulk density (apparent (loose) bulk density (L.B.D.) and tapped bulk density (T.B.D.)). It was further found by the invention that such a particulate precipitate aragonite calcium carbonate can be prepared by a process in which an aqueous calcium hydroxide slurry is reacted with a gas medium that comprises carbon dioxide. In order to obtain a composition of matter having the above parameters, the process operational parameters including the composition of the aqueous medium, the pH of the medium, the shear mixing speed, and others, are controlled to obtain this desired product. In accordance with one specific embodiment of the process, the product so formed becomes floated.

The term “effective refractive index”, is used herein to reflect the ability of a pigment to scatter light assuming that this property is determined only by its refractive index. It is a very useful term to describe cases at which the matrix around tested pigments is similar, or seems to be similar, and therefore any change in the ability of the tested pigment to scatter the light is contributed only by the pigment, irrespective of the real facts that caused it. The use of this term will become apparent by the Lorentz & Lorentz equation and the experimtal results in Example 19, hereinafter.

The particulate precipitated aragonite calcium carbonate can sorb substantial amounts of water or contain organic material. In order to obtain a true sense of the correct specific gravity, the particulate precipitated aragonite calcium carbonate of the invention may be dried, e.g. for 12 hours at about 120° C. Such dried product may then be ignited for about 8 hours at 500° C. Thus, according to a preferred embodiment, the particulate precipitated aragonite calcium carbonate of the invention, has a specific gravity below about 2.5 g/cm³, when determined under the following conditions:

(One) after drying for 12 hours at 120° C.; or

(Two) after drying for 12 hours at 120° C. and subsequently ignited for 8 hours at 500° C.

A product having the above characteristics has a hiding power that is not less than 90, which is an acceptable measure of a pigment's ability to disperse light or to opacity the medium into which it is immersed. A hiding power of above 90 is comparable to that of TiO₂ pigments. An example on the manner of determining the hiding power is given n Example 19A..

Said specific gravity is typically less than 2.3 g/cm³ and preferably even below about 2.1 g/cm³. A composition of matter of the invention having a specific gravity of less than 2.3 g/cm³ has a hiding power of at least 92 and that having a specific gravity of less than about 2.1 g/cm³ has a hiding power of at least 94.

According to one preferred embodiment of the invention, the process is carried out in the presence of or comprising the addition of a substance into an aqueous medium, said substance being selected from nonanoic acid, decanoic acid, undecanoic acid, dodecanoic acid, tetradecanoic acid, octadecanoicacid, and undecylenic acid, their carboxylate salts, their acid anhydrides, their esters, their acyl halides and their ketenes.

In the following, the term “reaction medium” will be used to denote the aqueous calcium hydroxide slurry used in the above process. Furthermore, an organic substance added into the reaction medium will be referred to herein as “active agent”.

In accordance with one embodiment, said active agent comprises one or more carboxylic acids of the formula RCOOH, wherein R may be a saturated or unsaturated, optionally substituted aliphatic group, e.g. a hydrocarbon group, that contains 7-21 carbon atoms or carboxylate salts, esters, anhydrides, acyl halides or ketenes thereof. By one example, the active agents comprise one or more carboxylic acids of formula CₙH_{2n±1}COOH, wherein n is 8-17, or their carboxylate salts, esters, anhydrides, acyl halides or their ketenes. By another example, the active agent comprises at least one carboxylic acid of the formula CH₃(CH₂)ₙCOOH, wherein n is 7-16, or their carboxylate salts, esters, anhydrides, acyl halides or their ketenes of the formula CH₃(CH₂)ₙ₋₁C═C═O.

The concentration of the active agent is typically within the range of 0.2 wt/ % and 10 wt. %, with the weight, in case the active agent is one of said carboxylate salts, esters, anhydrides, acyl halides or ketenes, based on the weight of the carboxylic acid with the formula RCOOH from which they are derived. The concentration of the calcium hydroxide in the reaction medium is typically within the range of about 3 to 30 wt. %, more preferably 4 to 20 wt. %.

The pH of the reaction medium is typically about 8 to about 11, preferably between about 9 to about 10. The process is typically carried out at a temperature within the range of about 60° to the boiling temperature of the reaction medium, preferably between about 80° C. and the boiling temperature of the reaction medium.

The process may be carried out in a semi-continuous (intermittent) mode, or, preferably, may be carried out in a continuous mode. The process is typically carried out under a high shear mixing, for example, with a mixture that comprises a rotor/stature or a rotor only, with the mixer peripheral speed (the tip speed) being preferably at least 5 m/sec.

In accordance with a particularly preferred embodiment of the invention, the process is carried out in a continuous mode of operation, with high shear mixing using a mixer that comprises a rotor/stature or a rotor only, and at a temperature that is about 90° C. In this preferred process, the active agent is included in a concentration ranging between about 0.2 to 10 wt. % and with the calcium hydroxide concentration being within the range of about 5 to about 15 wt. %. By a typical sequence, said active agent is premixed with the calcium hydroxide slurry prior to reaction with the carbon dioxide.

The novel composition of matter of the invention typically contains a carboxylic acid calcium salt in an amount between about 0.2 to about 10 wt. %, based on the weight of the carboxylic acid moiety. The specific gravity, while being typically less than about 2.5 g/cm³, is preferably less than about 2.0 g/cm³, more preferably less than about 1.8 g/cm³ and even more preferably less than about 1.5 g/cm³. A further characteristic of the composition of matter in accordance with one embodiment of the invention is that after having been previously dried, at about 120° C. for about 12 hours, has a further loss on drying at 300° C. for 8 hours of less than 10%, based on the weight of the calcium carbonate. Another characterizing feature of the composition of matter in accordance with the embodiment of the invention is that after having been previously dried, at about 120° C. for about 12 hours, it has a loss in weight after drying at about 300° C. for about 8 hours and/or after ignition at about 500° C. for about 8 hours, of less than about 10%.

The calcium salt of the carboxylic acid is typically a salt of the carboxylic acid having the formula RCOOH of which R contains, amongst other atoms, 7-21 carbon atoms, and more specifically the carboxylic acid having the formula CₙH_{2n±1}COOH, wherein n=8-17, in an amount between about 0.2 to about 10 wt. %, calculated based on the weight of the carboxylic acid moiety compared to the weight of the CaCO₃.

In accordance with another embodiment, said salt is a salt of the carboxylic acid having the following formula CH₃(CH₂)ₙCOOH. In accordance with some specific embodiments, the calcium salt is salt of a carboxylic acid being one or more of nonanoic acid, decanoic acid, undecanoic acid, dodecanoic acid, tetradecanoic acid, octadecanoic acid and undecylenic acid.

The composition of matter of the present invention can be used as a builder, an anticaking material, an encapsulant, an adsorbent, a thickening material, a sunscreen, a filler, an extender and particularly as a pigment for the detergent, pharmaceuticals, agrochemicals, plastics, adhesives, printing, coating (paint), paper, rubber, filtration, toiletries and many other industries. Thus, in accordance with further aspects of the present invention, there is provided a coating composition, a paper composition, a plastics composition, a rubber composition, an adsorbent composition, a powder detergent composition, a pharmaceutical composition, an agrochemical composition, a flavor composition, a fragrance composition, a food composition, a feed composition, a conductive composition, and a sunscreen composition, each of which comprises a particulate precipitated aragonite in accordance with the invention. For this purpose, such compositions may comprise, for example, substantially dry particulate precipitated aragonite, or particulate precipitated aragonite in aqueous dispersion.

The PCC of the present invention can be used in most (if not all) of the applications that the prior art particulate calcium carbonate is being used or proposed to be used (and quite probably in all of them). However, the PCC of the present invention manifests some advantages and unique properties over the prior art in the application that exploit its “porous” nature as an adsorbent for liquids, erg. in powders or detergent powders, in pharmaceuticals, in agrochemicals and in various household products like food and feed formulations; as an encapsulating agent for flavors and fragrances, pharmaceuticals and agrochemicals, and/or an anticaking agent, e.g. in powders or detergent powders; as an additive in pharmaceuticals, agrochemicals, food, and feed formulations; as a “light” component to reduce the bulk density of products, e.g. as a filler and/or a builder in powders or detergent powders; as a thickening material, e.g. in glues, sealants, adhesives, coatings (paints), and in paper); as a filler, as an extender; and, particularly, as a pigment e.g. in sunscreen formulations, plastics, adhesives, printing (inks), paints, paper (especially formulations for coating paper, and particularly for high gloss paper products), rubber, filtration, and many others).

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a schematic flow chart for production of particulate precipitated calcium carbonate according to the prior art.

FIG. 2 shows a schematic flow chart for production of a particulate precipitated aragonite, in accordance with an embodiment of the present invention.

FIG. 3 shows in schematic vertical section, a reactor/flotation cell for producing a particulate precipitated aragonite, in accordance with an embodiment of the present invention.

FIG. 4 shows a SEM picture of a substantially pure particulate precipitated aragonite, in accordance with an embodiment of the present invention.

FIG. 5 shows an XRD spectrum of a substantially pure particulate precipitated aragonite, in accordance with an embodiment of the present invention.

FIG. 6 shows a SEM picture of, ARP-76, a substantially pure particulate precipitated aragonite, in accordance with an embodiment of the present invention.

FIG. 7 shows an XRD spectrum of, ARP-76, a substantially pure particulate precipitated aragonite, in accordance with an embodiment of the present invention.

FIG. 8 shows a SEM picture of, ARP-70, a mixture of ˜50% particulate precipitated aragonite and ˜50% particulate precipitated calcite, in accordance with an embodiment of the present invention.

FIG. 9 shows an XRD spectrum of, ARP-70, a mixture of ˜50% particulate precipitated aragonite and ˜50% particulate precipitated calcite, in accordance with an embodiment of the present invention.

FIG. 10 shows the dependence of the hiding power of coatings made with two commercial TiO₂ pigments, and with the product of the presence invention vs the concentration of the pigments, respectively.

FIG. 11 shows a SEM picture (magnified ×100,000) of a substantially pure particulate precipitated aragonite, in accordance with an embodiment of the present invention.

FIG. 12 shows a SEM picture (magnified ×200,000) of a substantially pure particulate precipitated aragonite, in accordance with an embodiment of the present invention.

FIG. 13 shows a SEM picture (magnified ×110,000) of OPACARB A40 a commercial product of SMI.

FIG. 14 shows a SEM picture (magnified ×200,000) of OPACARB A40 a commercial product of SMI.

### DETAILED DESCRIPTION OF SPECIFIC EMBODIMENTS

In the process of the present invention, a slurry of calcium hydroxide in water and carbon dioxide gas or a carbon dioxide containing gas is reacted together in the presence of the active agent under stringent process conditions, to generate a particulate precipitated aragonite having unique properties.

The product of the present invention is characterized by its low production cost and by its unique physical properties (high opacity (namely, high effective refractive index), at least one of and preferably all of, low L.B.D. (<0.55 g/cm³), low T.B.D. (<0.70 g/cm³) and low specific gravity (<2.5 g/cm³)) and by its excellent chemical properties (hydrophobicity and resistance to weak acids), which make it particularly suitable as an adsorbent for liquids, an anticaking material, a thickening material, a builder, a filler, an extender and most of all as a pigment for the printing, coating (paint), paper, rubber, plastics, filtration, adhesives, sealants, pharmaceuticals, agrochemicals, food, feed, detergents and many other industries.

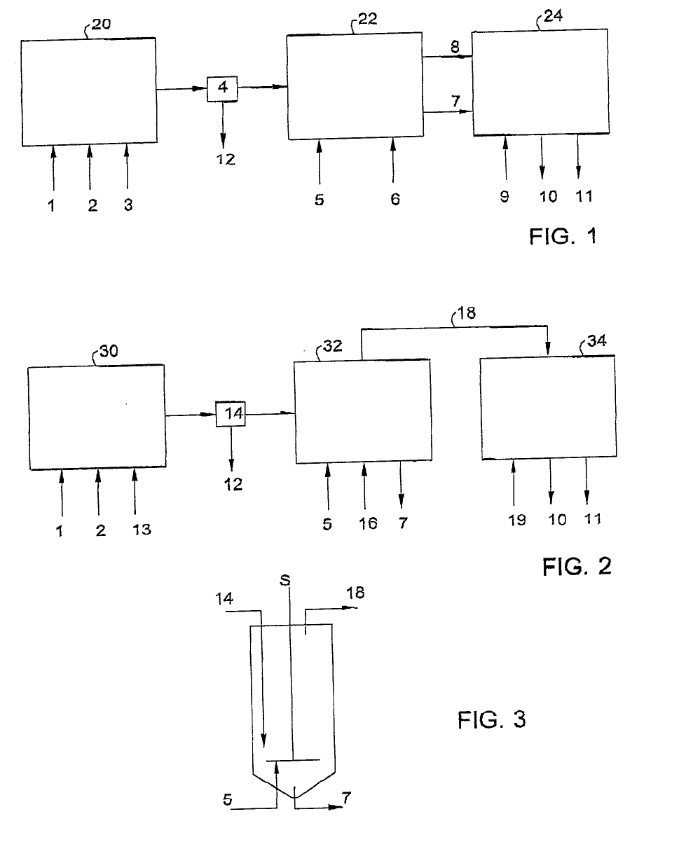

FIG. 1 shows a flow chart for production of particulate precipitated calcium carbonate according to the prior art. By contrast, in order to define the most suitable conditions to operate the carbonation stage of the present invention, a detailed description of parameters for the present process is given below. These also include some details of how to operate the upstream and downstream stages of the carbonation stage, as these may affect the final outcome (c.f. FIGS. 2 and 3).

In FIG. 1, which is a schematic representation of a prior art procedure for making a precipitated calcium carbonate, quicklime (CaO) and water, which react together giving slaked lime, are fed to reactor 20 via respective conduits 1 and 2, and optional additives such as aragonite calcium carbonate particles for seeding, phosphoric acids and salts, aluminum salts, oxides and hydroxide (other than CaO/Ca(OH)₂), chelating agents, dispersants, and surface active agents, may also be added at this stage via conduit 3. The initial product “milk of lime” (calcium hydroxide) is fed via filter or hydrocyclone 4 (large solid particles being removed at 12) into carbonator 22, to which there is also fed gaseous carbon dioxide (or a gas containing it) via conduit 5 and the aforementioned optional additives via conduit 6. The reaction product including any contaminants exits carbonator 22 as an runderflow via conduit 7 and/or an overflow via conduit 8, to further operations (at site 24) such as dewatering, grinding and coating; for such further operations there may be added optionally via conduit 9, e.g. dispersants, surface active agents, greases, silicon greases, long-chain carboxylic acids and their salts and esters, organic and inorganic pigments, powder metals, coal, carbon black or activated carbon, and/or dyeing agents. The filtrate and water vapors exit the system via conduit(s) 10, while the final product (which may be wet or dry and optionally post-treated) exits via conduit 11.

In FIG. 2, which is a schematic representation of a procedure for making a particulate precipitated aragonite in accordance with the present invention, quicklime (CaO) and water which react together giving slaked lime are fed to reactor 30 via respective conduits 1 and 2, and the present active agent (and optionally also additives such as phosphoric acids and salts, chelating agents, dispersants, and surface active agents) may be added at this stage via conduit 13. The initial product “milk of lime” (calcium hydroxide) together with active agent if added to 30 (and optional additives) is fed via filter or hydrocyclone 14 (large solid particles being removed at 12) into carbonator 32, to which there is also fed gaseous carbon dioxide (or a gas containing it) via conduit 5 and the active agent (and possibly the aforementioned optional additives) via conduit 16. It will be appreciated that the active agent may be added either to reactor 30 or to carbonator 32, or to both. Contaminants and liquid exit carbonator 32 as an underflow via conduit 7, whereas—owing to the fact that an embodiment of the present process includes simultaneous flotation—the desired product exits as an overflow via conduit 18, to further operations (at site 34) such as dewatering, grinding and coating; for such further operations there may be added optionally via conduit 19, e.g. dispersants, surface active agents, greases, silicon greases, long-chain carboxylic acids and their salts (including if desired those within the definition of the present active agents) and esters, organic and inorganic pigments, powder metals, coal, carbon black or activated carbon, and/or dyeing agents. The filtrate and water vapors exit the system via conduit(s) 10, while the final product (which may be wet or dry and optionally post-treated) exits via conduit 11.

Slaking of Quicklime

Though this operation is well known in the prior art, it is worthwhile to choose a preferred mode of operation, which is best, adapted to the process of the present invention. Thus, fresh slaked lime is preferably prepared in a continuous mode of operation, which enables operation of the downstream carbonation stage using low inventories and exploiting to its maximum the energy that is liberated in the reaction between the water and the CaO, before this precious energy is lost to the surroundings. The present invention desirably makes use of hiss energy to effect the step of carbonation of the aqueous calcium hydroxide slurry at relatively high temperatures, more preferably without cooling or heating, or in other words, without adding or subtracting energy, and thus utilizing only the energy liberated by the carbonation reaction together with the energy produced by a powerful mixing system. Once again, use of fresh and still warm milk of lime is preferred in the carbonation stage and this is more preferably effected, as mentioned above, in a continuous mode of operation, the temperature of the slaked lime being preferably maintained at about the temperature of the carbonation stage. However, in the alternative, a batch mode of operation may also be used for process of the present invention or the CaO can be introduced directly into the carbonator, as is demonstrated in the prior art.

Mixing of Quicklime

In some prior art processes it is recommended to use high shear mixers to slake the CaO with water. The process the present invention is quite tolerant to the kind of mixing, as long as the slaking reaction is complete and the maximum energy is liberated. Mixers that comprise rotor/stator mixing systems and mixers that comprise rotors only are suitable.

Purification of Slaked Lime Prior to Carbonation

There are numerous methods of purifying slaked lime before its utilization in the carbonation stage. Filtration by filters to remove large insoluble particles and/or separation of these particles by hydrocyclones are two efficient methods for this purpose. Usually, particles of greater diameter than 40 μm (up to 70 μm) are removed prior to the carbonation stage and the coarse particles can then be discarded or used in the construction industry, for example. The fine slurry is then ready for carbonation in the subsequent downstream stage Naturally, feeding CaO directly into the carbonator, as mentioned above, does not allow to make use of such purification methods.

Sources of CaCO₃/CaO

Many sources of CaCO₃/CaO are too contaminated to be used to produce, by known methods, a particulate precipitated aragonite for the printing, (inks), coating (paint), paper, rubber, plastics, filtration, adhesives and sealants, pharmaceuticals, household and personal care and other industries, and their main use is, as very inexpensive materials, in the construction industry. In accordance with the present invention many of these “impure” CaCO₃/CaO sources may be utilized to produce the particulate precipitated aragonite of the invention, of filler, extender and pigment grade. The present invention, as is manifested in the carbonation stage, is superior over any state of the art technology in salvaging CaCO₃ mines and turning them to profitable use, without changing greatly the state of the art methods for preparing the slaked lime.

Use of Additives

The state of the art technology for slaking quicklime includes adding a variety of additives into the milk of lime prior to the carbonation stage. According to the present invention, one of the preferred modes of operation is to add the active agent into the milk of lime prior to the carbonation reaction. As may readily be appreciated by those skilled in the art of producing precipitated calcium carbonate, it must be carefully checked that the other additives, if any are present in the milk of lime, do not interfere with the ability of the active agent to enhance formation of the particulate precipitated aragonite and to cause its flotation in the carbonation reactor. For instance, the use of 1 wt. % (based on the calcium carbonate) of phthalic acid or trimelitic acid with about 1 wt. % (based on the calcium carbonate) of one of the most potent active agents of the present invention, n-decanoic acid, cause the formation of mostly the particulate calcite polymorph in the carbonation stage, under the specific conditions that are described in the experimental section, instead of obtaining mostly the aragonite polymorph. In other cases, the additives may cause the formation of mixtures of various concentrations of particulate precipitated calcite and aragonite, instead of quite pure particulate precipitated aragonite calcium carbonate.

The Reaction/Carbonation Stage

As this stage, that is one of the characterizing features of the present invention, it is worthwhile to choose the mode of operation that suits it best. For example, although the use of aragonite particles for seeding is a recommended procedure in accordance with the prior art, it seems at the present time that this practice is unlikely to have any particular utility in the process of the present invention, since use of the active agent enables all desired product properties to be achieved.

As the most important functions of the active agent in the present invention are to catalyze the production of particulate precipitated aragonite, of improved physical and chemical properties and to cause its flotation in the carbonation reactor, all necessary measures should be taken in order to maximize these functions.

The Nature of the Active Agent and Its Origin

While the scope of the present invention is not to be regarded as limited by any theory, nevertheless, it is believed that the calcium salts of the carboxylic acids operate in practice as the functioning active agent in the present process. It should not be ruled out, however, that for example, other derivatives of such acids within the scope of the invention may participate in similar activity.

The above-mentioned calcium salts of the relevant acids may be used as raw materials in the present invention. However, other compounds, which undergo chemical transformations to form the active agent under the process conditions, also serve this purpose as raw materials in the production of the desired particulate precipitated aragonite.

In a particular embodiment of the invention which will serve here as an example, the active agent is selected from carboxylic acids of the general formula: CH₃(CH₂)ₙCOOH, where n=7-16, and including mixtures thereof. All these acids can be quite easily introduced into any of the production facilities. Pumping of these acids when their temperature is held above their melting points (e.g. above 60° C.) seems to be a very useful method to deliver the acids into the suitable production units. Under such conditions, these thermally stable acids are immediately converted into their respective calcium salts when they are mixed with the hot aqueous calcium hydroxide slurry or with the hot carbonation mixture at a pH above 7. As water is the only by-product of the reaction between the calcium hydroxide and the respective carboxylic acids, the use of these acids, as raw materials in the process of the present invention, seems to have no harmful side effect.

The respective acid anhydrides of the general formula: (CH₃(CH₂)ₙCO)₂O, including mixtures thereof, where n=7-16, are as good a source for the active agent, as the corresponding acids. However, the anhydrides are much less safe to handle and they are much more expensive than the respective acids.

The carboxylate salts of the acids of the general formula: CH₃(CH₂)ₙCOOH, including mixtures thereof, where n=7-16, can serve as raw materials in the process of the present invention, e.g. where the cations are selected from Na⁺, K⁺, NH₄⁺, Li⁺, Mg⁺⁺ and especially Ca⁺⁺, but, generally, the use of these salts does not appear to have any advantage over the free acids. On the contrary, the salts are usually more expensive, they are not as easy to handle on an industrial scale as the respective acids and, except the Ca⁺⁺ salts, all the other salts add cations that, so far as is presently known, are not required in the present process. The Mg⁺⁺ salts present a special case, as they leads to the formation of hydromagnesite and thereby to a dramatic rise of the surface area of the product, to its contamination and to a large increase in the water content in the wet filter cake. Therefore, in the process of the present invention only limited concentrations of this cation are allowed, i.e. <1 wt. %, based on the calcium hydroxide (this limitation is removed if it is desired to exploit the process of the present invention to produce hydromagnesite or mixtures of hydromagnesite and PCC of the present invention. On the contrary, then Mg⁺⁺ can also be introduced as other Mg salts or, preferably, as MgO/Mg(OH)₂).

Esters of the following general formula: CH₃(CH₂)ₙCOOR, where n=7-16 and R′ is an esterification radical such as alkyl, e.g. CH₃, C₂H₅, C₃H₇, etc., are also suitable candidates for the active agent in the process of the present invention. However, in order for these compounds to generate e.g. the corresponding calcium salts, they have to undergo a basic hydrolysis, which may preferably be done by premixing them in the hot and basic aqueous calcium hydroxide slurry in which they are hydrolyzed and thus converted to the respective Ca⁺⁺ salts. However, the use of these esters in the process of the present invention appears to be inferior to the use of the respective acids, for reasons, which will be self-evident to the skilled person.

Chemically equivalent to the other preferred active agents specifically mentioned above, are ketenes of the general formula: CH₃(CH₂)ₙ₋₁C═C═O, wherein n=7-16, and including mixtures thereof, behave in a similar manner and entail similar drawbacks, as for the acid anhydrides, as mentioned above.

Therefore, the acids of the general formula: CH₃(CH₂)ₙCOOH, wherein n=7-16, including mixtures thereof, are the presently preferred source for the active agent to be used in the process of the present invention. More specifically, decanoic acid (wherein n=8) is presently one of the most potent and preferred acids, as it leads to products of the present invention of which the content of the aragonite isomorph is the highest, under comparable conditions. Lauric acid (wherein n=10), myristic acid ((wherein n=12) or even stearic acid (wherein n=16), relatively abundant and less expensive raw materials, may be preferred in some other cases, in which the maximum content of the aragonite ismorph in the product is not critical or in cases in which controlled concentrations of the calcite isomorph in the product of the present invention may even be desirable.

It was also found out that undecylenic (or 10-undecenoic) acid (CH₂═CH(CH₂)₈COOH) is also a very potent active agent in the process of the present invention. Additionally a very large number of other carboxylic acids may be employed in the process of the invention. A person versed in the art should be able with simple and routine experimentation to bind other carboxylic acids to those mentioned above that may be used in accordance with the invention.

The Reactor/Carbonator/Flotation Cell

As already mentioned above, the carbonation stage can be conducted in any well-stirred reactor. However, due to the fact that the active agent is a unique material that can enhance the formation of the particulate precipitated aragonite of the present invention, in the reaction between aqueous calcium hydroxide slurries and carbon dioxide gas or a carbon dioxide containing gas, and also due to the fact that the active agent can cause this product to float, the presently preferred carbonators to be used in the process of the present invention are flotation cells.

These cells may be operated somewhat differently from the regular carbonators and the regular flotation cells, as both functions (carbonation and flotation) take place in the same production unit of the particulate precipitated aragonite, of the present invention. The exact set-up of these flotation cells can vary, as this will depend on, for example, the preferences of the skilled designer, the precise nature of the desired product, the quality of the aqueous calcium hydroxide slurries, etc. For example, a flotation cell like that depicted in FIG. 3, containing stator/rotor or rotor only S, is suitable for carrying out the inventive process, and of which the main features are as follows:

A. The stream of slaked lime (14) is preferably introduced near the inner circumference of the reactor and above the stirring blades.

B. The stream (5) of carbon dioxide gas or carbon dioxide containing gas is preferably introduced through suitable spargers at a point below the stirring blades, but still not too close to the bottom of the cell, to avoid excessive mixing near the outlet stream (7) of the contaminants and liquid.

C. The wet product and the gas are preferably discharged from the top (18) of the cell. The customary skimmer for skimming the product out of the flotation cell, and hydrocyclones for efficient product/gas separation, are not shown in FIG. 3.

Mode of Operation in the Carbonation Step

Continuous reaction/carbonation of the aqueous calcium hydroxide slurry with carbon dioxide gas or a carbon dioxide containing gas is the most suitable mode of operation for the present invention, especially because of the huge potential market for the produced particulate precipitated calcium carbonate, and particularly particulate precipitated aragonite.

Semi-continuous (intermittent) operations may also be used. However, as may be understood from the desirability of operating the process at its utmost efficiency, e.g. as a flotation operation, it is unlikely that an intermittent mode of operation can compete economically with the continuous mode of operation.

A “real” batch mode of operation, in which the milk of lime and the active agent are mixed together and carbon dioxide gas or a carbon dioxide containing gas is introduced to precipitate the desired product until the reaction mixture turns neutral (at about pH˜7), is less desirable, as the active agent is not efficient in catalyzing the formation of desired product, at the high initial pH characteristic of the batch mode of operation in this case, and/or because the active agent is adsorbed onto the surface of the first formed crystals of particulate precipitated calcium carbonate, where it is then “buried” under the subsequent PCC. In such circumstances, the active agent is very quickly depleted from the reaction zone, and the process of the invention, as such, is likely to become inoperable.

Temperature of the Carbonation Step

The prior art teaches producing a particulate precipitated aragonite, at a temperature range between 60° C. and the boiling temperature of the reaction mixture, at ambient pressure, and the present process is preferably conducted similarly, because lower temperatures favor the formation of calcite.

On the other hand, operating the process at a temperature as close as possible to the boiling point of the reaction mixture is presently particularly preferred, since these conditions give a product of relatively lower water content in the wet filter cake, which is a great advantage in many applications of the product.

While the present process may be operated at higher temperatures and pressures (since the active agent is stable under such conditions), this kind of operation is associated with serious technological problems that may adversely affect the whole economics of the process.

Concentration of Ca(OH)₂ Slurry in the Carbonation Step

The prior art method for producing a particulate precipitated aragonite, may be classified into three principle modes of operation. The first mode is operated at very low concentrations of the calcium hydroxide in water, and in some cases a clear solution of <1 wt. % calcium hydroxide is used. In the second mode, there are used aqueous calcium hydroxide slurries and additives to induce the formation of the desired particulate precipitated aragonite, albeit, at very low production rates. In the third mode, particulate precipitated aragonite is used for seeding, in order to improve production rates.

The present invention requires relatively high concentrations in the aqueous calcium hydroxide slurries and the production rates are very fast. Actually, at the range of very low concentrations of <2 wt. % (based on the calcium hydroxide) the present process may not “ignite” right away and under these circumstances no desirable “porous” product of the present invention is obtained, but rather, only precipitated calcite calcium carbonate particles, or mixtures of mainly such particles.

The present invention can use quite dense aqueous calcium hydroxide slurries of up to about 30 wt. % calcium hydroxide, but such dense slurries are very viscous and are very difficult to handle. Therefore, the preferred range of concentrations of the aqueous calcium hydroxide slurries, according to the present invention, are in the range between 4% and 20 wt. %, and more preferably between 5% and 15 wt. % calcium hydroxide. In these ranges, the viscosity of the reaction mixture permits smooth operation, while the energy maintained already in the feed of aqueous calcium hydroxide slurry (as discussed above), plus the energy liberated by the carbonation reaction, as well as the energy liberated by the mixing system, are sufficient to maintain the desired reaction temperature without any external heating or cooling.

Concentration of Active Agent in the Carbonation Step

To simplify the calculations of how much active agent is needed in the process and how much of it may be included in the product of the present invention, it is preferred to use the weights of the respective acids, since the carboxylate moieties differ from their respective acids by less than 1%. Therefore, in cases that suitable ketenes, esters, carboxylate salts, acid anhydrides and/or acyl halides are being used, the equivalent weight of the respective acid should be calculated, unless otherwise indicated. Moreover, there may be differences between the activities of the acids of the general formula e. g. CH₃(CH₂)ₙCOOH, wherein n=7-16, including mixtures thereof, their individual contribution to the total weight of the active agent may be calculated arithmetically, namely by adding the weight of each individual acid, as if these are of the same chemical entity. The difference between the molecular weights of the different acids (˜±30%) would not confuse a person skilled in the art who will be able to easily determine what is the exact amount of the relevant carboxylic acids that is necessary to operate the process in a manner, which is not sensitive to even larger variations of the concentrations of the active agent, namely, a preparation at above 30 wt. %, based on CaCO₃.

To determine the concentration range of the active agent in the present invention, it is important to be aware of the various functions of this agent in the production process and the effects that it produces in the final product.

Since the aqueous calcium hydroxide slurry is usually quite contaminated and the impurities are liable to affect performance of the active agent, the threshold (minimum) concentration of the active agent will vary, but is within the competence of a skilled person to determine, under any particular set of circumstances. Moreover, the threshold concentration will also vary with the kind of active carboxylic acids that will be used. In any case, it is desirable to avoid this threshold concentration at the carbonation stage, as this is a point of instability and would involve unnecessary risk to the desired objective. When considering use of a new feedstock of CaCO₃/CaO, laboratory experiments will reveal the minimum concentration of the active agent, which is necessary to start the production of the desirable particulate precipitated calcium carbonate, and particularly particulate precipitated aragonite, without any faults (vis-a-vis the pertinent CaCO₃/CaO feedstock). This value is expected to be in most cases above 0.2 wt. %, preferably within the range 0.4% to 3 wt. %, based on the calcium carbonate.

It is very important to note that this threshold concentration, discussed above, for catalyzing the production of particulate precipitated aragonite, of the present invention (˜0.2% wt. %, based on CaCO₃) is substantially above the threshold concentration that is required to cause the flotation of this product in aqueous solutions (˜0.02% wt. %, based on CaCO₃) and that by operating in the concentration range merely for a “proper” flotation process, the result achieved in accordance with the present invention is not achieved. Actually, the optimal physical and chemical properties of the particulate precipitated aragonite calcium carbonate, of the present invention, are attained at above 100 fold of this concentration (˜2-3 wt. %, based on CaCO₃).

Other factors may indicate use of even higher concentrations of the active agent in the production process of the present invention. For instance, coating the surface of the particulate precipitated aragonite, with a predetermined rather thick layer of the active agent, in situ, in a carbonator/flotation cell, may require quite high concentrations of this material, which may exceed 5%, 10% and even 15 wt. %, based on CaCO₃, in order to produce good surface coated hydrophobic and acid resistant particulate precipitated aragonite (e.g. for master batches). Naturally, at such high active agent concentrations, the cost component of the coating should then be compared to the alternative possibilities of downstream coating, which are also available in the prior art, as well as in the present invention (c.f. FIGS. 1 and 2, respectively). Another serious reason to avoid operating the process at too low concentrations, is the fact that the chemical and physical properties of the product, and especially its optical properties and specific gravity, which are quite interdependent, are dramatically affected by the concentration of the active agent.

In between the upper limit and the threshold limit of the concentration of the active agent in the process of the present invention, the optimum concentration should also be determined by one skilled in this art, either vis-a-vis the quality of the CaCO₃/CaO, or whenever the properties of the product are to be changed. The active agent is not an expensive material, but still it may throw an economical burden on the total cost of the final product due to the fact that even quite pure particulate precipitated aragonite is a relatively inexpensive material.

Intuitively, the concentration of 10 wt. %, based on the calcium carbonate, seems to be an economical upper limit of the active agent, while 0.2 wt. %, wt; based on the CaCO₃, seems to be its threshold (minimum) concentration.

Carbon Dioxide in the Carbonation Step

Use of carbon dioxide gas or a carbon dioxide containing gas is well known in the prior art methods for producing precipitated calcium carbonate particles. The process of the present invention is similar in this respect to the prior art processes that operate with substantially pure carbon dioxide gas as well as with mixtures of carbon dioxide with up to about 92 v % inert gases (e.g. air). At lower concentrations of the carbon dioxide in the feed gas (<8 v %), however, the efficiency of the process may be too low, mainly, due to the cooling effect of the excessive gas.

In order to understand how to control the process of the present invention, It is worthwhile to describe the major effects that are observed at the two limits, namely, when using “rich” feed gas of about 100% carbon dioxide on the one hand and using “lean” feed gas of about 8 v % carbon dioxide on the other hand. It was found that “rich” carbon dioxide gas feed leads to a PCC, of the present invention, that gives rise to products of much higher gloss than those that are produced from the PCC, of the present invention, that are produced with a “lean” carbon dioxde feed under similar production conditions. Namely, the gloss of the final (consumer) products can easily be fine-tuned by just choosing the right CO₂/inert gas (air) ratio. This fact may be exploited, especially, in using the product of the present invention in formulations that are intended to be used e. g. in the coating industry, which requires quite often low gloss products and in the paper industry for coating paper and obtaining a desirable product of high gloss.

Another phenomenon that is observed when using “lean” feed gas is that it leads to a PCC, of the present invention, of lower specific gravity and of higher hiding power, compared to a PCC, of the present invention, that is produced with “rich” feed gas under similar conditions. That, in turn, allows to include carboxylic acids within the patent range, which otherwise could not meet the constrains that were set up to determine which carboxylic acid is within the borders of this invention and can be used as an active agent to produce the product of the present invention. For instance, under the conditions of the screening test (Example 1; that is described hereinafter), Lauric acid could not be considered active agents (its product was considered “Calcite” as its crystallographic purity (aragonite/(aragonite+calcite)) was only 20%-25% and its specific gravity (in tall oil; after drying it at 120° C. for twelve hours) was only 2.54 g/cm³. When using a “lean” feed gas of 26% CO₂ (by volume) the specific gravity of the product decrease dramatically to 1.78 g/cm³. Therefore, based on the too high specific gravity values that were obtained for lauric acid, for palmitic acid and for stearic acid, myristic acid was not considered, at that time, to be a viable candidate to catalyze the process of the present invention and, therefore, due to constraints of time, it was not tested and not included in Table 1. The use of very “lean” gas feed allows to sort out and use much more carboxylic acids (lauric acid, myristic acid and even stearic acid) to serve as the active agents of the process of the present invention, using the simple and straight forward methods that were developed herein

Additives in the Process

The process of the present invention is quite self-sufficient and requires only the active agent in suitable quantities, as discussed above. The active agent can be introduced preferably already premixed with the aqueous calcium hydroxide slurry, or alternatively (or additionally) it can be introduced directly into the carbonator. The active agent can also be used downstream the carbonation stage, but that, naturally, has no effect on the production of the particulate precipitated calcium carbonate, and particularly the particulate precipitated aragonite, in the carbonator.

It appears that the active agent has a surprising affinity to the aragonite, which is unlikely to be adversely affected by the presence of other additives. Consequently, additives like phosphoric acids and water soluble salts thereof, can be used in the present invention to modify the product properties by increasing the aspect ratio of the thus formed acicular crystals; polyacrylates, polyacrylamides and some short-chain carboxylic acids can be used to modify the rheology of the product mixtures and allow operation at higher calcium hydroxide concentrations and, consequently, at higher throughputs; chelating agents can be used to convert heavy metals into water-soluble species and once again lead to super-pure products; metal powders and carbon black may be introduced to obtain electrically conductive powders; soluble aluminum salts may affect the shape of the calcite particles; and magnesium salts or preferably MgO/Mg(OH)₂ may lead to hydromagnesite. The prior art has many examples of additives that are used to achieve improved particulate calcium carbonate products. These additives and many others may, potentially, be used in the product (process) of the present invention. Some of the additives, when used under the right process conditions, may serve as said active agents.

It is nevertheless prudent to check carefully the effect that well known additives of the prior art may have on the action of the active agent, but in most cases the active agent will be the dominant catalyst for the purpose of the present invention and, therefore, such additives can usually be introduced at various stages of the process, as is customary in the prior art (c.f. FIGS. 1 and 2).

The Mixing System

The preference for high shear mixing in this process is well known in the relevant art. The mixers may be a rotor/stator type or a rotor only type. Usually, the latter one is used to produce relatively larger product particles, while the rotor/stator type leads to much higher attrition of the acicular crystals. On the other hand, the rotor/stator type may allow a more efficient dispersion of the gas bubbles, thereby improving the quality of the product. The skilled operator will utilize the preferred mixing system for working or enhancing the present process. The type of mixers and the rotor speed should be optimized according to the desired carbonation performance and the desired product characteristics.

The lower limit of the rotor speed (hereinafter—“Tip Speed” or “Peripheral Speed”) is known in the prior art. A preference for a minimum tip speed of about 5 m/sec., to effect the formation of desired product is not unusual in this field.

The upper limit of the rotor speed is determined by the mixer technology, cost of the specific mixer, the nature of the desired product and the energy that is to be used. For instance, the higher the rotor speed, the lower may be the reaction time (in a continuous process, the reaction time is termed HUT (Hold Up Time) and it is calculated as follows: HUT=V (the carbonator volume)/F(the discharge rate of the product mixture out of the carbonator)). This in turn may lead to small particles. A skilled person in this art will know how to optimize the kind of mixers and rotor speeds above the minimal peripheral speed, which is preferably 5 m/sec.

The Reaction Duration in the Reactor/Carbonator/Flotation Cell

As already mentioned above, the carbonation step is preferably conducted in a continuous mode of operation. In such a case, “reaction duration” is hardly relevant, but we can calculate the HUT (Hold Up Time), which lies essentially within the range between 5 minutes and 180 minutes. At below the lower limit of the HUT the yields may be too low and the PSD (Particle Size Distribution) of the product may be too small, while at the upper limit of the HUT the process throughput may be too low, the yields may be excellent and the PSD may be too small, because of excessive attrition of the product in the flotation cell. Once again, the skilled person will be able to determine by experiment, suitable working parameters vis-a-vis the desired product properties and to optimize its quality and cost.

The Specific Gravity and the Hiding Power (H.P.) of the Particulate Precipitated Calcium Carbonate of the Present Invention

While the present invention is not limited by any theory, it seems that trapped gas (air) in the product account for the unusual optical properties (hiding power, contrast ratio and opacity that can be used interchangeably) observed in the present product. The specific gravity (S.G.) and the Hiding Power (H.P.) of the PCC of the present invention are measured for the following three major reasons: (a) to distinguish the product of the present invention from the products of the prior art; (b) to distinguish the process of the present invention from the processes of the prior art; and (c) to control and optimize the process and the product of the present invention.

Specific Gravity (S.G.)

The specific gravity of calcite and of aragonite are well documented in the literature and are always well above 2.5 g/cm³. However, measurement of the S.G. of the present product, as well as the PCC/GCC products of the prior art which may be coated with hydrophobic coatings (e.g. calcium salts of long-chain carboxylic acids), may lead to erroneous results, if it is not done properly. On one hand, superficially adhering air bubbles should be thoroughly removed, and on the other hand, it should not be conducted by evacuation of most of the air from the tiny “pores”, “voids” or “microvoids” that are deliberately produced so that the gas will stay trapped in these small voids and manifest the creation of a novel particulate PCC, and more specifically, a novel particulate aragonite PCC.

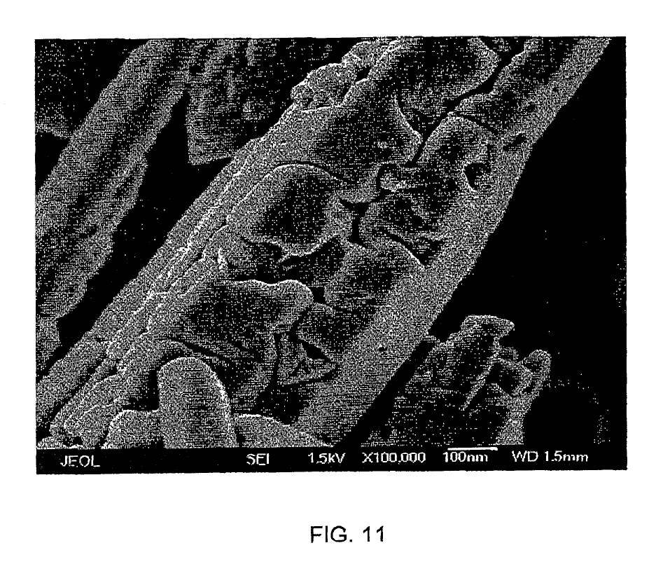

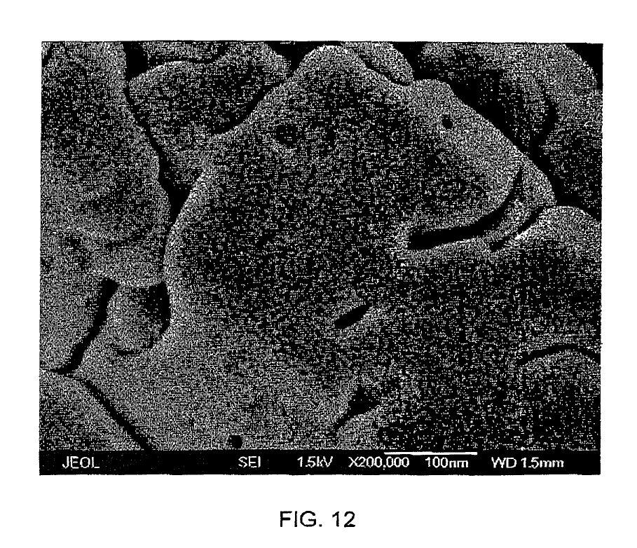

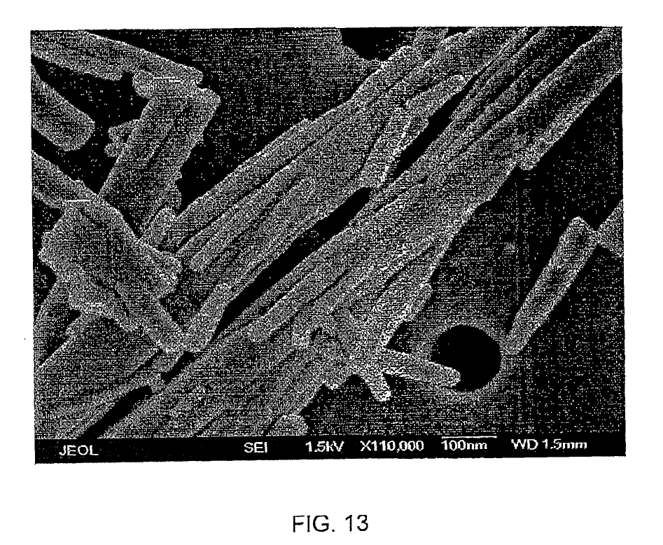

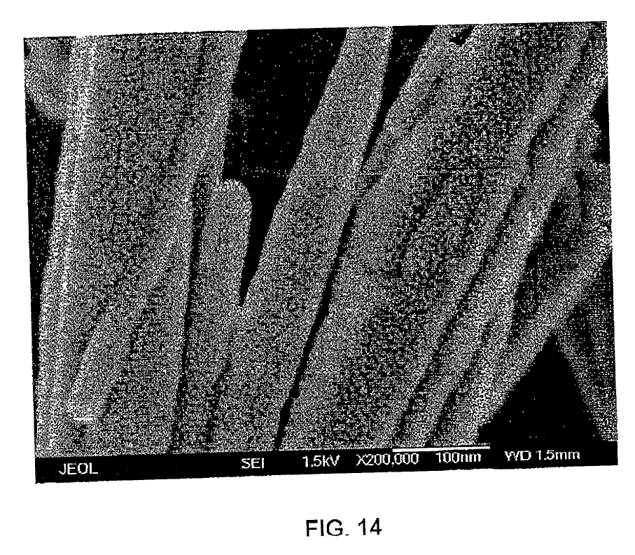

Example 14 (D) is presented in order to show an incorrect way to determine the S.G. of the product of the present invention. The SEM FIGS. 11 and 12 of a product of the present invention furnish the detailed microstructure of the product of the present art and makes it clear now that a unique and novel product was created and that this product deserves to be handled by suitable or new “tools”. In comparison, the SEM FIGS. 13 and 14 of OPACARB A40, a commercial product of SMI distributor demonstrate why routine determination methods of S.G., as well as the methods that are described in e.g. Examples 14(A) and 14(C) will lead to similar results—definitely S.G. values >2.5 g/cm³.

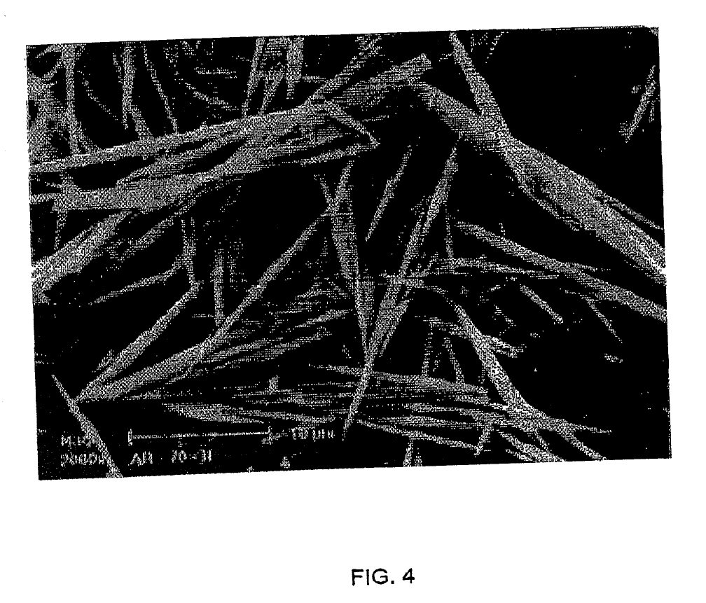

In order to better differentiate the products of the present invention from those of the prior art, while using very simple and inexpensive methods, the S.G. of the dry products may be determined in various oils, which simulate the practical environment in which the PCC/GCC particles are customarily used, at least in their major applications. This determination of S.G. may be carried out on the dry products as produced, e.g. as is described in Example 14(A), and/or after igniting them at 500° C. for eight hours, e.g. as is described in Example 14(C) herein. The S.G. values of the dried PPC/GCC particles should reflect their real properies under conditions in which they are to be used in most cases, while the S.G. values determined after calcination should reveal whether the S.G. values of the dried products indicate significant structural differences from prior art products. However, now that the SEM FIGS. 11 and 12 revealed that indeed a novel product with a unique microstructure was created, which was hidden in the SEM FIGS. 4, 6 and 8, there is not much need for the S.G. values after calcination.

Similar considerations apply to the determination as to whether the process that produced such a product is a process according to an embodiment of the present invention, as the specific gravity values for products of the prior art (calcite, as well as, aragonite) are always >2.5 g/cm³ (even >2.65 g/cm³), while the products according to a particular embodiment of the present invention are characterized by their specific gravity values <2.5 g/cm³ (preferably <2.3 g/cm³ and even more preferably <2.1 g/cm³).

As already mentioned, bubbles of gas may adhere superficially to the surface of the PCC particles, but these bubbles are forced to leave by mixing and sonicating so that the S.G. may be disputed only in the vicinity of 2.5 g/cm³. However, in the “real” cases, at which the S.G. values are below 2.3 g/cm³, there is no doubt anymore whether these values reflect the product of the present invention. At any rate, this situation lasts only until these PCC particles are subjected to high shear forces (e. g. in the processes of making coatings, inks and papers), which causes the separation of these gas bubbles, unless they are sealed or hidden in tiny and narrow “pores”, “voids” or “microvoids” and they can not leave their positions during their entire downstream processing steps.

As the microstructure of the PCC particles of the present invention can now be observed by SEM at a magnification of ×100,000 to ×200,000, it is quite clear why the movement of trapped air bubbles is slow and can happen under severe forces, only. Any attempt to use the regular gas phase pycnometer measurements to determine the specific gravity of commercial PCC particles, as well as the PCC of the present invention, will lead to values well above 2.5 g/cm³, irrespective of the kind and source of these calcium carbonate products or the kind of treatment that these samples received prior to the specific gravity analyses (c.f Example 14(D)). Namely, using such a practice would have resulted in totally overlooking the present invention. However, conducting specific gravity analyses of the PCC particles of the present invention in liquids like water, oleic acid (>97%), cold pressed edible olive oil, refined edible sunflower oil, refined edible corn oil, refined edible soybeans oil, refined canola oil, and tall oil, leads to values <2.5 g/cm³ (preferably <2.3 g/cm³ and more preferably <2.1 g/cm³), as shown e.g. in Example 14(H). Similar measurements of the specific gravity of commercially available GCC (calcite) and PCC (calcite and aragonite) gave always rise to values that were >2.5 g/cm³ (even >2.6 g/cm³ and even >2.7 g/cm³). When the measurements of the specific gravity were conducted in water, the specific gravity of the products of the present invention were <2.5 g/cm³, but however, when a small amount of sodium dioctylsulfosuccinate (2%) was added to the slurry, the specific gravity of the PCC particles increased quite fast and ended up at values >2.7 g/cm³ (such a phenomenon could not be observed in the case of the prior art GCC or PCC products). This phenomenon of penetration of liquids into the “pores” of the product of the present invention has a lot of benefits, but when this happens on preparing the usual stable slurries of PCC (of >50 wt %) by mixing the PCC, water and a suitable dispersants, it results in a very thick, high viscose mass. At the preparation stage, PCC slurries made of a product of the prior art look, superficially, quite similar to those that are made of the product of the present invention, but this superficial appearance may mislead. For instance, measuring the specific gravity (S.G.) of the PCC particles of the prior art will result in values that are >2.5 g/cm³, while the S.G. values of the PCC particles of the present invention will be considerably lower. Moreover, the addition of a wetting agent, like the sodium dioctylsulfosuccinate, to these slurries will reveal a much more dramatic behavior. Namely, such slurries of the PCC particles of the prior art may not be affected much, but the slurries made of the PCC particles of the present invention will turn into a dense and thick high-viscose mass and the S.G. values of the particles therein will increase to >2.7 g/cm³, due to the penetration of the aqueous phase into the “pores” of the PCC particles.

Due to the fact that the “pores” in the products of the present invention are not closed to gases and to some liquids, it is important to avoid the customary gas pycnometer specific gravity (S. G.) measurements when analyzing the product of the present invention, and rather follow the exact instructions of how to do it (c.f. in Example 14(A) and especially in Example 14(C)). These measurements reveal best the desired properties of the novel products of the present invention and of the process of the present invention and give a close picture of how these PCC particles are going to improve the final (consumer) products, due to their unique property—“porosity”.

Hiding Power (H.P.)

The refractive index is the most important parameter of a pigment when comparing its ability to opacity e.g. coatings, paper and plastics to other pigments. The hiding power, the contrast ratio and the opacity (contrary to whiteness and brightnes) serve best to correlate the refractive indices of different pigments, as their measurements take care to minimize the the optical effects that are being introduced by their respective different particle size distribution (PSD) and their different shapes.

The H.P. of coatings that are made with single commercial pigments in Example 19(A) are compared with that of the product of the present invention. The results are given in Example 19(B). Pigments in this experiment include top quality commercial TiO₂ pigments, top quality commercial CaCO₃ pigments and a precipitated particulate CaCO₃ of the present invention. As the coatings in this Example and the H.P. measurements are done under similar conditions, the differences among the various H.P. values reflect, mainly, the differences among the refractive indices of the respective pigments (the Lorentz-Lorentz expression of M=[(nₚ/nₒ²−1]/[(nₚ/nₒ)²+2]; where nₚ is the refractive index of the respective pigment and nₒ is the refractive index of the medium in which the respective pigments are immersed, is probably one of the best ways to correlate the H.P. of coatings—c.f. Pigment Handbook (Vol. I-III; Edited by T. C. Patton; John Wiley & Sons, New York (1973); Vol. III; Pages 289-290. A graphic illustration of the linear relation H.P. vs M² is given in FIG. 2 on Page 290).

Accordingly, the H.P. values of the coatings, in Example 19(B), that contain top quality TiO₂ pigment are expected to be much higher than any of those coatings that are made with CaCO₃ pigment, only (for TiO₂ (n=2.76—Rutile; in Vol. I; Page 3 of the above Handbook) >> for CaCO₃ (n=1.530, 1.681 and 1.685—Orthorombic Aragonite; in Vol. I; Page 119 of the above Handbook)≅for CaCO₃ (n=1.486, 1.658; Calcite; in Vol. I; Page 119 of the above Handbook)).

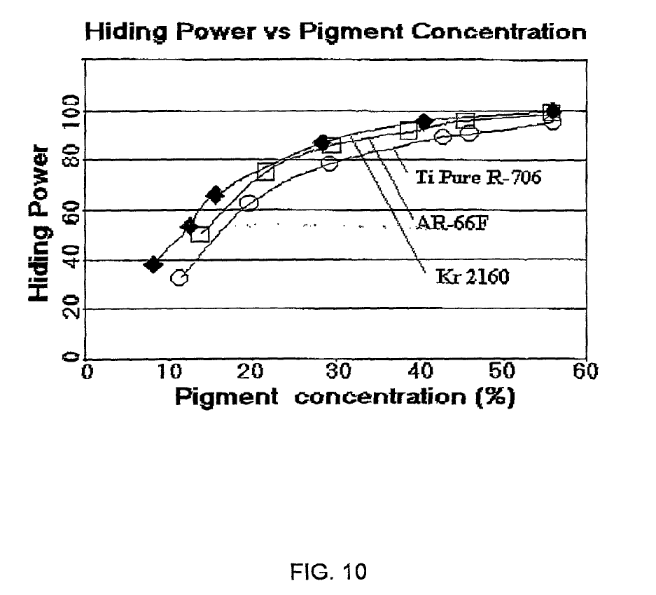

It is surprising that the hiding power of the coating made with the product of the present invention is very close to the results obtained for the TiO₂ of Kronos and DuPont (FIG. 10), even though no optimization has been done yet to get the best of the present invention. It is even more surprising to see that the H.P. of the coating made with the product of the present invention is higher than that of the DuPont product (c.f the graphic presentation of these results in FIG. 10).

A similar comparison of the top quality commercial CaCO₃ pigments to the TiO₂ pigments in Example 19 justifies the summary article of A. Cole (mentioned above), claiming that there was no white mineral powder that challenged TiO₂ pigments (at the time that the article was published on May, 2001).

This is a manifestation of the effect of trapped gas (air) in the “pores”, “voids”, “microvoids” or just “indentations” that are present in the product of the present invention and which are clearly shown in the SEM FIGS. 11 and 12. It is worth noting that no such “indentations” and not such a microstructure can be observed in the SEM FIGS. 13 and 14 of OPACARB A40, and therefore, it is quite clear that this top quality commercial product of SM, as well as the other CaCO₃ pigments, of which the specific gravity values are higher than 2.5 g/cm³ can not compete with the TiO₂ pigments (the S.G. results can be found in Example 14).

The outstanding optical properties of the product of the present invention are attributed to the trapped air bubbles, which can be measured by the simple methods that are given in Example 14(A), 14(C) and 14(E) and that there is not yet a CaCO₃ pigment that can challenge now either the TiO₂ pigments or the product of the present invention.

The present invention will now be described in more detail by way of Examples, which are presented for illustration purposes only and are not be construed restrictively.

Experimental:

Raw Materials:

A. All raw materials were purchased from Aldrich, unless otherwise specified.

B. Ethyl decanoate was prepared by reacting decanoyl chloride with ethanol in the presence of triethylamine at about 50° C. After about 3 hours the product was washed with water to remove water-soluble residues and it was then dried at about 50° C. under a vacuum of about 30 mm/Hg.

C. Sodium decanoate was prepared by thoroughly mixing decanoic acid with 2% aqueous NaOH at about 70° C. until the pH passed 10.

D. Potassium decanoate was prepared by thoroughly mixing decanoic acid with 2% aqueous KOH at about 70° C. until the pH passed 10.

E. CaO(1)—of Arad, Israel.

F. CaO(2)—of Shfeya, Israel.

G. Commercial PCC—Aragonite; of Specialty Minerals Inc. (SMI); Opacarb® A40.

H. CO₂—Cylinders of 100% pure compressed gas of Mifalay Hamzan Ltd., Haifa.

I. Tall Oil (Sylvatal 20S) of Arizona Chemical, USA.

J. Ultrafine stearic acid coated GCC—Omya UFT 95 ex Omya-a-Pluess-Staufer—Switzerland.

K. A commercial ultrafine stearic acid coated—Ultrapflex PCC ex SMI—USA.

L. A commercial ultrafine talc—Ultratalc 609 ex SMI—USA.

M. Isostearic Acid—Emersol 875 ex Henkel—Germany.

N. Anise Alcohol (ex Koffolk—Israel).

O. Hexabromocyclododecane (Syntex HBCD ex Albermarle—USA).

P. NeendX (ex Albermarle—USA).

Q. Diazinon (Diazol ex Makhteshim-Agan—Israel).

R. The Paint Constituents:

Nopco NDW of Henkel

Cellosize QP 15000 (hydroxy ethyl cellulose) of Union Carbide

Disperse One (45% N.V.) of Tambour, Israel

Synperonic NP10 of ICI

TiO₂ (Ti Pure R-706; a product of Du Pont (organic treated)).

TiO₂ (Kronos 2160) of Kronos (However, similar TiO₂ pigments, like Tioxide R-TC90 and Tioxide TR92, of which their D₅₀=220 nm±20 nm may serve equally well)

Synthetic sodium aluminum silicate (p820) of Degussa

Kaolin clay (D₅₀=3.1 micron) of Engelhard

CaCO₃ powder (d₅₀=3.5 microns) of Polychrom, Israel—“Girulite-8”

Talc (D₅₀=12.3 micron) of Lusenac Val Chisone

Copolymer vinyl acetate acrylate emulsion (55% N.V.) of Cerafon, Israel

Butyl diglycol acetate of Union Carbide

Kathon LXE of Rohm & Haas

Ammonia (25%) of Frutarom, Israel

Antioxidant (irganox B225 ex Ciba Specialty Chemicals—Switzerland)

Lubricant (Wax PE 520 ex Hoechst-Celanese—USA)

Polypropylene copolymer (Capilene-TR50 ex Carmel Olefins—Israel)

Dispex N-40 of Allied Colloids

Thickener (TT 615; a product of Akzo)

Resin (Acronal 290D; a product of BASF)

Opacarb A40, Uncoated; a top quality PPC Aragonite product of SMI

Instruments and Accessories:

1. pH meter/controller; Jenco; Model 3671; Made in China.

2. pH electrode; Hanna Industries; type HI 1131B (Glass Probe).

3. Thermometer; Jenco Model 3671; Made in China.

4. Peristaltic pump; Watson-Marlow; Model 505u (variable speed).

5. Agitator; Ika; Model RW-20 (variable speed).

6. Dissolver; Hsiangtal; Model HD-550; Made in Taiwan

7. Ultra-turrax® T50; Ika; rotor d=3.8 cm; stator d=4 cm.

8. Disk type rotor of d=12 cm.

9. Disk type rotor of d=8 cm.

10. Saw-blade type rotor of d=9 cm.

11. Saw-blade type rotor of d=4.8 cm.

12. Hydrocyclone 2″; Mozely; P=50 psi; vortex finder=11 mm; spigot=6.4 mm.

13. Vacuum pump; Vacuumbrand GmbH; Model MD 4C.

14. Buchner+filter cloth with 8-10 μm pores.

15. XRD (X-Rays Diffractometer); Siemens D-500 for the crystallographic phases.

16. SEM (Scanning Electron Microscope); Jeol 5400 for the shapes of the particles.

17. Colorimeter; Hunterlab D25-PC2 for whiteness measurements.

18. Colorimeter; ACS instrument (Applied Color Systems).

19. Ultrasonic bath (10 l); Selecta, Spain—“ULTRASONS”.

20. Ultrasonic cleaners (baths) of limited power (<100 Amp.Volt.) e.g. P-08890-01/06 ex Cole Parmer—USA.

21. Analytical Balance; Shekel Ltd., Israel.

22. BPLC Analyzer; Waters HPLC Analyzer (Detector 486+Autosampler 717+Pump 510+millenium Software).

23. HPLC Column; Phenomenex C18(250 mm×4.3 mm; 5 μm Particle size).

24. AccPyc 1330 ex Micromeritics—USA.

25. Glossmeter (Minigloss 101N ex Sheen Instruments—England).

26. Reflectometer (Ref. 310 Sheen-Opac ex Sheen Instrument—England).

27. Hiding Power chart (Ref 301/2A ex Sheen instruments Ltd.)

28. Twin-screw compounder (L/D=24 ex Dr. Collin—Germany).

29. Injection machine (25 t ex Dr. Boy—Germany).

30. Screen-shaker (Rotap Model RX-29-10 ex W. S. Tyler Inc.—USA).

31. GC-MS for trace analysis—of HP Model 5890/5971

32. Hegmann (Sheens apparatus for fine grinding measurement gauge ref 501/100).

33. Stormer (Sheen 480 ex Sheen instruments Ltd.).

34. The high resolution SEM pictures, FIGS. 11-14, were taken on a JEOL, JSM-6700 FESEM, a high resolution scanning electron microscope with a field emission (FE) source, after depositing Pt onto the PCC samples at high vacuum.

PREPARATION I—Preparation of Aqueous Calcium Hydroxide Slurries:

The aqueous calcium hydroxide slurry was prepared in the laboratory in a batch mode of operation as follows: 40 kg of tap water were introduced into a 50 l. stainless steel 316 reactor that was equipped with a steam heated jacket, a thermometer and with the Hsiangtal Dissolver with a rotor of d=12 cm. The Dissolver was operated at 200 rpm, 4 kg of CaO (Shfeya) were added to the reactor during less than 10 minutes and the slurry was allowed to stir for 10-80 minutes. At that time the temperature rose to above 60° C. and when it reached its maximal temperature at 80-90° C., the mixture was ready for its purification prior to the carbonation stage, as follows:

One. The slurry passed a stainless steel 316 screen to remove particles of d>2 mm, and

Two. The filtered slurry passed a hydrocyclone to remove particles of d>50 μm.

Notes:

At this point the warm aqueous calcium hydroxide slurry was ready for its use in the carbonation stage and its temperature was maintained at a preset value by heating the slurry in the above reactor in order to control the temperature in the carbonator.

The potential active agent(s) and any optional additives could be blended into the warm slurry at a preset concentration before the purification steps a. and b. or thereafter.

This batch mode of operation is used only in the laboratory tests. The production plant is intended to be operated under a continuous mode of operation, as is discussed herein.

PREPARATION II—Preparation of Aqueous Calcium Hydroxide Slurries:

PREPARATION I was repeated using CaO of Arad, a substantially purer raw material than that of Shfeya (the respective whitenesses are >95% and ˜88%).

### EXAMPLE 1

### Screening Test for the Potential Active Agents

Possible active agents were investigated by producing particulate precipitated calcium carbonate according to the following procedure:

2 kg tap water were added to a 3.2 1. stainless steel 316 reactor (of inner diameter d=15 cm and length ˜18 cm), equipped with a steam heated jacket, a pH electrode, a thermometer and the Hsiangtal Dissolver with a saw-blade rotor of d=4.8 cm (c.f. FIG. 3). The Dissolver was operated at a preset speed and carbon dioxide gas or a carbon dioxide containing gas and the aqueous calcium hydroxide slurry of PREPARATION I, containing already the active agent, were fed simultaneously into the reactor, while maintaining the pH, the temperature and the production rate at preset values. The product was collected at the top of the reactor, and the impurities were discharged from the bottom of the reactor (naturally, the product exited from the bottom of the reactor when the experimental active agent did not lead to a particulate precipitated aragonite and to its flotation).

The first 10 l. of resulting slurry were discarded. The residual slurry was collected and it was filtered through a filter-cloth on the Buchner using a vacuum pump to dewater the product. The filter cake was dried for 12 hours at 120° C. and the crystallographic morphologies and the shapes of the crystals of the precipitated calcite and/or aragonite calcium carbonate particles were determined using XRD and SEM analyses, respectively. The results are shown in the Table 1, below.

The Process Set Points—Continuous Mode of Operation:

1. Rotor Speed=4000 rpm (Tip Speed˜10 m/sec.).

2. pH=9.5.

3. Temperature=85° C.

4. Carbon dioxide flow rate=180 L.P.H. (liters/hour).

5. Aqueous calcium hydroxide slurry (of Shfeya) −10% (wt)=˜6 L.P.H. (to maintain the preset pH value).

6. Potential active agent concentration=1 wt. %, based on CaCO₃.

TABLE 1
the results of EXAMPLE 1

| | | Number of | |
|---|---|---|---|
| Test # | Active Agent | Carbons | Product (Isomorph) |
| 1 | ropionic acid | 3 | Calcite |
| 2 | actic acid | 3 | Calcite |
| 3 | yruvic acid | 3 | Calcite |
| 4 | crylic acid | 3 | Calcite |
| 5 | ethoxyacetic acid. | 3 | Calcite |
| 6 | ethacrylic acid. | 4 | Calcite |
| 7 | utanoic acid | 4 | Calcite |
| 8 | entanoic acid | 5 | Calcite |
| 9 | exanoic acid | 6 | Calcite |
| 10 | eptanoic acid | 7 | Calcite |
| 11 | ctanoic acid | 8 | Calcite |
| 12 | hthalic acid | 8 | Calcite |
| 13 | erephthalic acid | 8 | Calcite |
| 14 | -Ethylhexanoic acid | 8 | Calcite |
| 15 | onanoic acid | 9 | Aragonite |
| 16 | onanoic acid* | 9 | Aragonite |
| 17 | zelaic acid | 9 | Calcite |
| 18 | rimelitic acid | 9 | Calcite |
| 19 | ecanoic acid | 10 | Aragonite |
| 20 | ecanoic acid* | 10 | Aragonite |
| 21 | odium decanoate | 10 | Aragonite |
| 22 | otassium decanoate | 10 | Aragonite |
| 23 | thyl decanoate | 12 | Aragonite |
| 24 | ecanoyl chloride | 10 | Aragonite |
| 25 | ecanoic acid anhydride | 20 | Aragonite |
| 26 | ndecanoic acid | 11 | Aragonite |
| 27 | ndecanoic acid* | 11 | Aragonite |
| 28 | -Butylbenzoic acid | 11 | Calcite |
| 29 | odecanoic acid** | 12 | Calcite |
| 30 | almitic acid | 16 | Calcite |
| 31 | tearic acid | 18 | Calcite |
| 32 | leic acid | 18 | Calcite |
| 33 | gCl₂ | — | Calcite |
| 34 | 1Cl₃ | — | Calcite |
| 35 | ₁₂H₂₅C₆H₄SO₃H (LABSA) | 18 | Calcite |
| *Was pumped continuously and directly into the carbonator. | | | |
| **This experiment led to mostly calcite (of a crystallographic purity (aragonite/(aragonite + calcite)) ˜20%-25%) and to a specific gravity (S. G.) = 2.54 g/cm3 (measured according to Example 14A), which is outside the limits of the present invention (S. G. < 2.5 g/cm3). However, | | | |
| # the use of CO2 containing gas (26% by volume CO2 and 74% by volume Air) and 1.5% dodecanoic acid in an experiment similar to that described in Example 10, led to a product of mostly aragonite (of a crystallographic purity (aragonite: (aragonite + calcite)) > 50%) and to a specific | | | |
| # gravity (S. G.) = 1.78 g/cm3 (measured according to Example 14A). | | | |

### EXAMPLE 2

### A Screening Test for Interfering Compounds

EXAMPLE 1 was repeated, except that in all the experiments 1% (wt; based on the calcium carbonate) decanoic acid was premixed in the aqueous calcium hydroxide slurry feed and in each experiment an additional experimental active agent was added to study its effect on the activity of the decanoic acid. The results are shown in Table 2, below.

The Process Set Points—Continuous Mode of Operation

1. Rotor Speed=4000 rpm (Tip Speed˜10 in/sec.)

2. pH=9.5.

3. Temperature=85° C.

4. Carbon dioxide flow rate=180 L.P.H. (liters/hour).

5. Aqueous calcium hydroxide slurry (of Shfeya) −10% (wt)=˜6 L.P.H. (to maintain the preset pH value).

6. Active agents concentrations=1 wt. % decanoic acid+1 wt. % potential active agent based on CaCO₃.

TABLE 2
the results of EXAMPLE 2

| | | Number of | |
|---|---|---|---|
| Test # | Active Agent | Carbons | Product (Isomorph) |
| 1 | Propionic acid | 3 | Aragonite |
| 2 | Lactic acid | 3 | Aragonite |
| 3 | Pyruvic acid | 3 | Aragonite |
| 4 | Acrylic acid | 3 | Aragonite |
| 5 | Methoxyacetic acid | 3 | Aragonite |
| 6 | Methacrylic acid | 4 | Aragonite |
| 7 | Butanoic acid | 4 | Aragonite |
| 8 | Pentanoic acid | 5 | Aragonite |
| 9 | Hexanoic acid | 6 | Aragonite |
| 10 | Heptanoic acid | 7 | Aragonite |
| 11 | Octanoic acid | 8 | Aragonite |
| 12 | Phthalic acid | 8 | Calcite |
| 13 | 2-Ethylhexanoic acid | 8 | Aragonite |
| 14 | Nonanoic acid | 9 | Aragonite |
| 15 | Azelaic acid | 9 | Aragonite |
| 16 | Trimelitic acid | 9 | Calcite |
| 17 | Decanoic acid | 10 | Aragonite |
| 18 | Undecanoic acid | 11 | Aragonite |
| 19 | 4-ButylBenzoic acid | 11 | Aragonite |
| 20 | Dodecanoic acid | 12 | Aragonite |
| 21 | Palmitic acid | 16 | Aragonite |
| 22 | Stearic acid | 18 | Aragonite |
| 23 | Oleic acid | 18 | Aragonite |
| 24 | MgCl₂ | — | Aragonite |
| 25 | AlCl₃ | — | Aragonite |
| 26 | C₁₂H₂₅C₆H₄SO₃H (LABSA) | 18 | Aragonite |

### EXAMPLE 3

### A Batch Mode of Operation

A batch mode of operation, of which parameters were as close as possible to those of EXAMPLE 1, was attempted. Only particulate precipitated calcite of rhombohedral shape was obtained. No particulate precipitated aragonite could be obtained when using decanoic acid or any other active agent that was mentioned as being effective in EXAMPLE 1. The experiment was conducted as follows:

The active agents were investigated by producing precipitated calcium carbonate particles according to the following procedure:

2 kg aqueous calcium hydroxide slurry, containing already the respective active agent (c.f. EXAMPLE I) were added to the 3.2 1. stainless steel 316 reactor of EXAMPLE 1. The Dissolver was operated at 4000 rpm, the temperature was maintained at 85° C. and the production rate was determined by controlling the feed rate of the carbon dioxide gas. The carbonation was stopped after about 20-30 minutes, when the pH reached 7. The product mixture was then removed from the reactor through its bottom outlet.

The resulting slurry was filtered through a filter cloth on the Buchner using a vacuum pump to dewater the product. The filter cake was dried for 12 hours at 120° C. and the crystallographic morphologies and the shapes of the crystals of the precipitated calcite particles were determined using XRD and SEM analyses, respectively. As mentioned above, no precipitated aragonite particles were obtained.

The Process Set Points—Batch Mode of Operation:

1. Rotor Speed 4000 rpm (Tip Speed˜10 m/sec.).

2. pH=A˜14→7.

3. Temperature=85° C.

4. Carbon dioxide flow rate=180 L.P.H. (liters/hour).

5. Aqueous calcium hydroxide slurry (of Shfeya) −10% (wt)=2 kg.

6. Potential active agent concentration 1 wt. %, based on CaCO₃.

### EXAMPLE 4

### Parametric Studies—The Effect of the Temperature

Similar experiments to EXAMPLE 1 were conducted using decanoic acid only. The results are as follows:

The Process Set Points—Continuous Mode of Operation:

1. Rotor Speed=4800 rpm (Tip Speed˜12 m/sec.).

2. pH=9.5.

3. Temperature=variable.

4. Carbon dioxide flow rate=180 L.P.H. (liters/hour).

5. Aqueous calcium hydroxide slurry (of Shfeya) −10 wt. %=˜6 L.P.H. (to maintain the preset pH value).

6. Active agent concentration=decanoic acid; 0.5 wt. %, based on CaCO₃.

TABLE 4
the results of EXAMPLE 5

| | | Mineralogical Phase |
|---|---|---|
| Test # | pH | XRD |
| 1 | 10 | Aragonite |
| 2 | 9.5 | Aragonite |
| 3 | 9 | Aragonite |
| 4 | 8.5 | Aragonite |
| 5 | 8.0 | Calcite |
| 6 | 7.0 | Calcite |

### EXAMPLE 6

### Parametric Studies—Concentration Effect of the Active Agent

Similar experiments to EXAMPLE 1 were conducted using decanoic acid only. The results are as follows:

The Process Set Points—Continuous Mode of Operation:

1. Rotor Speed=4800 rpm (Tip Speed˜12 m/sec.).

2. pH=9.5

3. Temperature=87° C.

4. Carbon dioxide flow rate=180 L.P.H. (liters/hour).

5. Aqueous calcium hydroxide slurry (of Shfeya) −10% (wt)=˜6 L.P.H. (to maintain the preset pH value).

6. Active agent concentration=decanoic acid; variable wt. %; based on CaCO₃.

TABLE 5
the results of EXAMPLE 6

| | Decanoic acid | Mineralogical Phase |
|---|---|---|
| Test # | % (wt) | XRD |
| 1 | 1.0 | Aragonite |
| 2 | 0.5 | Aragonite |
| 3 | 0.3 | Aragonite + Calcite* |
| 4 | 0.2 | Aragonite + Calcite* |
| 5 | 0.1 | Calcite |
| *A crystallographic purity (aragonite/(aragonite + calcite)) <90%. | | |
| Note: | | |
| Though the present invention is especially aimed at obtaining substantially pure particulate precipitated aragonite calcium carbonate of crystallographic purity (aragonite phase/(aragonite phase + calcite phase)) ≧90% and even >95%, there are still applications that can utilize mixtures of these isomorphs where such crystallographic purity is <90%, and such mixtures are within the scope of the present invention. In such | | |
| # cases the boundary conditions of the present invention (c. f. Tests #3 and #4, above) may still be used. | | |

### EXAMPLE 7

### Parametric Studies—Concentration Effect of the Ca(OH)₂

Similar experiments to EXAMPLE 1 were conducted using decanoic acid only. The results are as follows:

The Process Set Points—Continuous Mode of Operation:

1. Rotor Speed=4800 rpm (Tip Speed˜12 m/sec.).

2. pH=9.5.

3. Temperature=87° C.

4. Carbon dioxide flow rate=180 L.P.H. (liters/hour)

5. Aqueous calcium hydroxide slurry (of Shfeya) −variable wt. %=˜variable L.P.H. (to maintain the preset pH value).

6. Active agent concentration=decanoic acid; 0.5 wt. % based on CaCO₃.

TABLE 6
the results of EXAMPLE 7

| | Solids in | |
|---|---|---|
| | Slaked Lime | Mineralogical Phase |
| Test # | % (wt) | XRD |
| 1 | 8 | Aragonite |
| 2 | 4 | Aragonite |
| 3 | 3 | Aragonite + Calcite* |
| 4 | 2 | Aragonite + Calcite* |
| 5 | 1 | Calcite |

*A crystallographic purity (aragonite: (aragonite + calcite)) < 90% and c.f. the above note at the end of Example 6.

### EXAMPLE 8

### Parametric Studies—Rotor Speed Effect

Similar experiments to EXAMPLE 1 were conducted using decanoic acid only. The results are as follows:

The Process Set Points—Continuous Mode of Operation

1. Rotor Speed=variable rpm (Tip Speed˜variable).

2. pH=9.5.

3. Temperature 87° C.

4. Carbon dioxide flow rate 180 L.P.H (liters/hour).

5. Aqueous calcium hydroxide slurry (of Shfeya) −10 wt. %=˜6 L.P.H. (to maintain the preset pH value).

6. Active agent concentration=decanoic acid; 0.5 wt. % based on CaCO₃.

TABLE 7
the results of EXAMPLE 8

| | Rotor Speed | Tip Speed | Mineralogical Phase |
|---|---|---|---|
| Test # | rpm | m/sec. | XRD |
| 1 | 10000 | 25 | Aragonite |
| 2 | 4800 | 12 | Aragonite |
| 3 | 2000 | 5 | Aragonite + Calcite* |
| 4 | 1000 | 2.5 | Calcite |

*A crystallographic purity (aragonite: (aragonite + calcite)) < 90% and c.f. the above note at the end of Example 6.

### EXAMPLE 9

### Parametric Studies—Effect of the CO₂ Flow Rate (F.R.)

Similar experiments to EXAMPLE 1 were conducted using decanoic acid only. The results are as follows:

The Process Set Points—Continuous Mode of Operation:

1. Rotor Speed=4800 rpm (Tip Speed˜12 m/sec.).

2. pH=9.5.

3. Temperature 87° C.

4. Carbon dioxide flow rate=variable L.P.H. (liters/hour)

5. Aqueous calcium hydroxide slurry (of Shfeya) −variable wt. %=˜variable L.P.H. (to maintain the preset pH value).

6. Active agent concentration=decanoic acid; 0.5 wt. % based on CaCO₃.

TABLE 8
the results of EXAMPLE 9

| | CO₂ Flow Rate | Mineralogical Phase |
|---|---|---|
| Test # | L.P.H. | XRD |
| 1 | 240 | Aragonite |
| 2 | 180 | Aragonite |
| 3 | 120 | Aragonite |

### EXAMPLE 10

### Parametric Studies—Effect of the CO₂/Air Ratio

Similar experiments to EXAMPLE 1 were conducted using decanoic acid only. The results are as follows:

The Process Set Points—Continuous Mode of Operation:

1. Rotor Speed 4800 rpm (Tip Speed˜12 m/sec.).

2. pH=9.5.

3. Temperature=87° C.

4. Carbon dioxide flow rate=180 L.P.H. (liters/hour).

5. Aqueous calcium hydroxide slury (of Shfeya) −10% (wt)=˜6 L.P.H. (to maintain the preset pH value).

6. Active agent concentration=decanoic acid; 0.5 wt. %; based on CaCO₃.

7. Air=variable.

TABLE 9
the results of EXAMPLE 10

| | | Mineralogical Phase |
|---|---|---|
| Test # | Air/CO₂ | XRD |
| 1 | 0 | Aragonite |
| 2 | 0.33 | Aragonite |
| 3 | 0.66 | Aragonite |

### EXAMPLE 11

### The Effect of Active Agent on Content of the Wet Filter Cake

Similar experiments to EXAMPLE 1 were conducted using decanoic acid only. The content of CaCO₃ in the wet filter cake was determined after drying 12 hours at 120° C. Relatively pure (aragonite phase/(aragonite phase+calcite phase)) ≧95% and dry precipitated acicular aragonite calcium carbonate particles were obtained. The results are as follows:

The Process Set Points—Continuous Mode of Operation:

1. Rotor Speed=4800 rpm (Tip Speed˜12 m/sec.).

2. pH=9.5.

3. Temperature=90° C.

4. Carbon dioxide flow rate=180 L.P.H. (liters/hour).

5. Aqueous calcium hydroxide slurry (of Shfeya) −10 wt. %=˜6 L.P.H. (to maintain the preset pH value).

6. Active agent concentration=decanoic acid; 0.7; 1.0; 2.0 wt. %; based on CaCO₃.

TABLE 10
the results of EXAMPLE 11

| | Dosage | Product | CaCO₃ | Crystallographic |
|---|---|---|---|---|
| Test # | % (wt) | (Isomorph) | % (wt)* | Purity** |
| 1 | 0.7 | Aragonite | >80 | ≧95% |
| 2 | 1.0 | Aragonite | >80 | ≧95% |
| 3 | 2.0 | Aragonite | >80 | ≧95% |

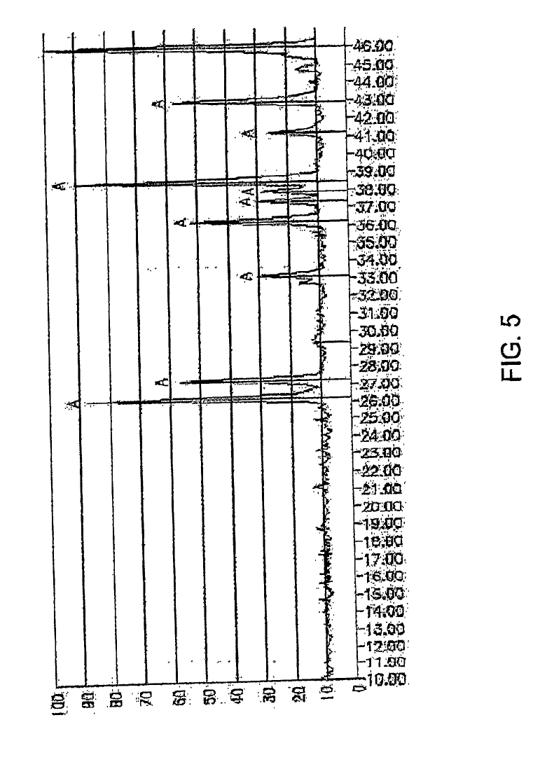

*% (wt.) CaCO₃ = 100 × wt. of dry filter cake/wt. of wet filter cake
**As determined by the XRD analyses (for Test #2 - c.f. FIGS. 4 and 5)

Note:

By choosing relatively standard conditions for the present process, it is possible to reduce the water content in the wet filter cake below 20 wt. %.

### EXAMPLE 12

### The Effect of the Active Agent on the Resistivity to Acids

Similar experiments to EXAMPLE 1 were conducted using decanoic acid only, the resistivity of the dry samples to acidic aqueous solutions being determined as follows. A 5 l. solution of HCl in water at pH=3.5 was prepared for all the following experiments, so as to assure equal starting experimental conditions. 100 ml of this HCl solution were poured into a 100 ml graduated cylinder, 5 g of precipitated CaCO₃ particles were added and the pH was measured after 20 minutes. Evolution of CO₂ was observed visually, as was the behavior of the commercial sample #C, the calcite #BM-37, and it was found that the aragonite samples of the present invention (#12-3, #12-4, #12-5) were markedly different. It is worthwhile to note that sample #12-5 produced few bubbles that did not detach from the surface of the precipitated aragonite particles. The results are as follows:

The Process Set Points—Continuous Mode of Operation:

1. Rotor Speed=5200 rpm (Tip Speed˜13 m/sec.).

2. pH=9.5.

3. Temperature 90° C.

4. Carbon dioxide flow rate=180 L.P.H. (liters/hour).

5. Aqueous calcium hydroxide slurry (of Shfeya) −10 wt. %=˜6 L.P.H. (to maintain the preset pH value).

6. Active agent concentration=decanoic acid; 0.7%; 1.0%; 2% wt. % based on CaCO₃.

TABLE 11
the results of EXAMPLE 12

| | | Active agent | Product | pH after 20 | |
|---|---|---|---|---|---|
| Test # | Sample # | (wt. %) | (Isomorph) | mins. | Note |
| 1 | C* | Unknown | Aragonite | >6 | Violent |
| | | | | | Evolution of CO₂ |
| 2 | BM-37** | 1.0 | Calcite | >5 | Evolution of CO₂ |
| 3 | 12-3 | 0.7 | Aragonite | <4 | Slight Evolution of CO₂ |
| 4 | 12-4 | 1.0 | Aragonite | <4 | Slight Evolution of CO₂ |
| 5 | 12-5 | 2.0 | Aragonite | <4 | No Evolution of CO₂ |

*Commercial PCC - Aragonite; of Specialty Minerals Inc. (SMI); Opacarb ® A40
**This sample was taken from the batch mode of operation in EXAMPLE 3
Note:
By choosing relatively standard conditions for the present process, it is possible to increase the resistance of the product towards acids by using quite low concentrations of the active agent and obtain excellent product for the paper industry of which processes are acidic and for the coating industry for durable paints for acidic environments.

### EXAMPLE 13

### Effect of Raw Material/Process on Whiteness of the Product

EXAMPLE 1 and EXAMPLE 3 were conducted using the aqueous calcium hydroxide slurries of PREPARATION I and of PREPARATION II for comparison. The whitenesses of the products are compared.

The results are as follows:

The Process Set Points—Continuous Mode of Operation:

1. Rotor Speed 4000 rpm (Tip Speed˜10 m/sec.).

2. pH=9.5.

3. Temperature=85° C.

4. Carbon dioxide flow rate=180 L.P.H. (liters/hour)

5. Aqueous calcium hydroxide slurry (of Arad/Shfeya) −10 wt. %=˜6 L.P.H. (to maintain the preset pH value).

6. Active agent concentration=decanoic acid; 1 wt. % based on CaCO₃.

The Process Set Points—Batch Mode of Operation:

1. Rotor Speed=4000 rpm (Tip Speed˜10 m/sec.).

2. pH=14→7.

3. Temperature=85° C.

4. Carbon dioxide flow rate=180 L.P.H. (liters/hour).

5. Aqueous calcium hydroxide slurry (of Arad/Shfeya) −10 wt. %=2 kg.

6. Active agent concentration=decanoic acid 1 wt. % based on CaCO₃.

TABLE 12
the results of EXAMPLE 13

| CaO of Arad (whiteness = >95%) | | CaO of Shfeya (whiteness = ˜88%) | |
|---|---|---|---|
| Continuous | Batch | Continuous | Batch |
| AR-83A | BM37A | AR-83 | BM37 |
| Aragonite | Calcite | Aragonite | Calcite |
| CaCO₃ | CaCO₃ | CaCO₃ | CaCO₃ |
| Whiteness = | Whiteness = | Whiteness = | Whiteness = |
| 98-9% | 97-8% | 97-9% | 92-5% |

Notes:
1. When the raw material (CaO) is relatively pure, the whiteness of the products (AR-83A and BM37A) is not (and should not be) much different. However, when the CaO is relatively impure, the whiteness of the precipitated aragonite particles (AR-83) is dramatically higher than the corresponding calcite (BM37), due to the unique effect of the process of the present invention.
2. The whiteness of the precipitated particulate aragonite obtained according to the present process attains top quality, independently of the calcium hydroxide source.

### EXAMPLE 14

### Effect of the Active Agent/Process on the Specific Gravity (S.T.) of Precipitated Particulate Calcium Carbonate

EXAMPLE 1 was repeated using the aqueous calcium hydroxide slurry of PREPARATION I, except that the concentration of decanoic acid was gradually increased.

(A) Determination of the Specific Gravity (S.G.) in Tall Oil of a Product Dried at 120° C.

1. The wet filter cake of the CaCO₃ sample was dried for 12 hours at 120° C. to remove all free water.

2. A weighed quantity of the dry CaCO₃ sample (Wc)+a weighed quantity of tall oil (Wo) (Density of 0.93 g/cm³) were introduced into a 1 1. glass beaker.

3. The mixture was stirred with the Hasiangtal HD-550 Dissolver for 10 minutes, at 4000 rpm (using a saw-blade rotor of d=4.8 cm).

4. The slurry was poured into a 250 ml graduated glass settling column and was sonicated gently in an ultrasound bath for 20 minutes, until all the visible trapped bubbles were released from the surface of the PCC particles. In order not to destroy the structure of the “porous” product of the present invention while sonicating it, thereby leading to higher S.G. values, the use of ultrasonic cleaners (baths) of limited power (<100 Amp.Volt.) e.g. P-08890-01/06 ex Cole Panner—USA, is recommended.

5. The settling column was then evaluated at 20-22° C. for:

(a) the volume of the slurry—V

(b) the total net weight of the slurry—W

Based on the above measurements, the following was calculated:

(1) from the equation: D=W/V g/cm³, the density of the slurry;

(2) from the equation

1/D=[Wc(Wo+Wc)]/S.G.+[Wo(Wo+Wc)]/0.93,

the S.G. of the CaCO₃ sample was calculated.

6. The loose bulk density (L.B.D.) of the dry powder was measured using a balance and a graduated cylinder (c. f. the exact procedure in EXAMPLE 14(F)).

The Process Set Points—Continuous Mode of Operation:

1. Rotor Speed=4000 rpm (Tip Speed˜10 m/sec.)

2. pH=9.5.

3. Temperature=85° C.

4. Carbon dioxide flow rate=180 L.P.H. (liters/hour).

5. Aqueous calcium hydroxide slurry (of Shfeya) −10 wt. %=˜6 L.P.H. (to maintain the preset pH value).

6. Active agent concentration=decanoic acid; 0.5; 1; 2; 5; 2; 1 wt. % based on CaCO₃.

The results are as follows:

TABLE 13
the results of EXAMPLE 14 (A)

| | | | | Mineralogical | | Loose |
|---|---|---|---|---|---|---|
| Test | Sample | Active | Dosage | Phase | S.G.^{†} | B.D.^{†} |
| # | Code | agent | (wt. %) | XRD | g/cm³ | g/cm³ |
| | Natural | — | — | Calcite | 2.63 | 0.65 |
| | CaCO₃ | | | | | |
| 2 | BM-37* | decanoic | 1 | Calcite | 2.54 | 0.37^{BM} |
| | | acid | | | | |
| 3 | C** | N.A. | N.A. | Aragonite | 2.56 | 0.54 |
| 4 | AR-81 | decanoic | 0.5 | Aragonite | 2.02 | 0.31 |
| | | acid | | | | |
| 5 | AR-83 | decanoic | 1 | Aragonite | 1.90 | 0.30 |
| | | acid | | | | |
| 6 | AR-118 | decanoic | 2 | Aragonite | 1.75 | 0.25 |
| | | acid | | | | |
| 7 | AR-119 | decanoic | 5*** | Aragonite | 1.67 | 0.29 |
| | | acid | | | | |
| 8 | AR-135 | Nonanoic | 1 | Aragonite | 1.88 | 0.31 |
| | | acid | | | | |
| 9 | AR-120 | Decanoic | 2\hat{ } | Aragonite | 1.72 | 0.23 |
| | | acid | | | | |
| *The sample was taken from the batch mode of operation in - EXAMPLE 3. | | | | | | |
| **Commercial PCC - Aragonite; of Specialty Minerals Inc. (SMI); Opacarb ® A40. | | | | | | |
| ***5 g of AR-119 were dissolved in a 10% HCl solution. The decanoic acid was extracted with 1,2-dichloroethane. HPLC analysis using a C18 colunm revealed 4.93% (wt.; based on the calcium carbonate) of this acid in the sample. | | | | | | |
| \hat{ }50 ppm of phosphoric acid were used in addition to the decanoic acid to increase the aspect ratio of the acicular aragonite. | | | | | | |
| ^{†}A dry powder after drying for 12 hours at 120° C. | | | | | | |
| ^{BM}The relatively low L.B.D. of this product should not be compared to the other L.B.D. of Tests = 3-9, as BM-37 is a calcite form, while the others are acicular aragonite form. | | | | | | |
| N.A.—Not available. | | | | | | |
| Notes: | | | | | | |
| 1. The determination of a specific gravity (S.G.) of particulate precipitated aragonite calcium carbonate of the present invention, in a range below 2.5 g/cm³ (after drying at 120° C. for twelve hours as described above, as well as after ignition of the dried material at 500° C. for eight hours) is actually an important and decisive test to consider if the technology that was used is under the domain of the present invention. | | | | | | |
| 2. Only the inclusion of gas (probably, as tiny bubbles or “blisters”) in closed pores can account for the dramatic reduction of the S.G. of particulate precipitated aragonite calcium carbonate of the present invention. The L.O.D. and the L.O.I. in the latter tests (c. f. (B) and (C), respectively) do not leave many logical choices to account for this phenomenon. Also, this is in accordance with the facts i. that the product of the present invention is obtained | | | | | | |
| #under flotation conditions, and ii. paints, in which the particulate precipitated aragonite calcium carbonate of the present invention was used, are probably due to the high effective refractive index of this product of the present invention, which is much higher than that expected of similar products that are produced according to the prior art (c. f. data collected in EXAMPLE 15 and the comparison made to paint formulations that were based on raw materials of the prior art). | | | | | | |

3. The idea of using porous particles, to increase their effective refractive index in coatings, is not new. For instance, Rohm & Haas produces a series of such products, e. g. Ropaque ® OP96 and Ropaque ® OP3000. However, these particles are of an organic polymeric nature of which cost and adaptation to the environment is not to be compared with precipitated calcium carbonate particles.

(B) Determination of the Specific Gravity (S.G.) in Tall Oil of a Product Calcined at 300° C.,

1. The wet filter cake of the CaCO₃ sample was dried for 12 hours at 120° C. to remove all the free water.

2. The weighed dry sample was heated for 8 hours at 300° C. The loss on drying (L.O.D.) was then determined.

3. The S.G. of the heated powder was measured as above (c. f. (A)).

4. The loose bulk density (L.B.D.) of the dry powder was measured using a balance and a graduated cylinder (c. f. the exact procedure in EXAMPLE 14(F)).

The results are as follows:

TABLE 14
the results of EXAMPLE 14 (B)

| | | | | L.O.D. | | Loose |
|---|---|---|---|---|---|---|
| Test | Sample | Active | Dosage | wt. % | S.G.^{†} | B.D.^{†} |
| # | Code | agent | (wt. %) | 300° C. | g/cm³ | g/cm³ |
| 10 | Natural | — | — | 0.10 | 2.63 | 0.651 |
| | CaCO₃ | | | | | |
| 11 | BM-37* | decanoic | 1 | 2.21 | 2.64 | 0.372 |
| | | acid | | | | |
| 12 | C** | N.A. | N.A. | 1.3 | 2.63 | 0.54 |
| 13 | AR-81 | decanoic | 0.5 | 0.83 | 2.19 | 0.255 |
| | | acid | | | | |
| 14 | AR-83 | decanoic | 1 | 0.89 | 2.11 | 0.265 |
| | | acid | | | | |
| 15 | AR-118 | decanoic | 2 | 2.32 | 2.03 | 0.200 |
| | | acid | | | | |
| 16 | AR-119 | decanoic | 5 | 5.73 | 2.01 | 0.200 |
| | | acid | | | | |
| 17 | AR-135 | nonanoic | 1 | 0.95 | 2.12 | 0.238 |
| | | acid | | | | |
| 18 | AR-120 | decanoic | 2\hat{ } | 2.27 | 2.02 | 0.235 |
| | | acid | | | | |

*The sample was taken from the batch mode of operation in - EXAMPLE 3.
**Commercial PCC - Aragonite; of Specialty Minerals Inc. (SMI); Opacarb ® A40.
\hat{ }50 ppm of phosphoric acid were used in addition to the decanoic acid.
^{†}A dry powder after heating for 8 hours at 300° C.
N.A.—Not available.

(C) Determination of the Specific Gravity (S.G.) in Tall Oil of a Product Calcined at 500° C.

1. The wet filter cake of the CaCO₃ sample was dried for 12 hours at 120° C. to remove all the free water.

2. The dry sample was calcined for 8 hours at 500° C. The loss on ignition (L.O.I.) was then determined.

3. The S.G. of the calcined powder was measured as in EXAMPLE 14(A), above.

4. The loose bulk density (L.B.D.) of the dry powder was measured using a balance and a graduated cylinder (c.f. the exact procedure in EXAMPLE 14(F)).

The results are as follows:

TABLE 15
the results of EXAMPLE 14 (C)

| | | | L.O.I. | | Loose |
|---|---|---|---|---|---|
| | Sample | Dosage | % (wt) | S.G.^{†} | B.D.^{†} |
| Test # | Code | % (wt) | 500° C.\hat{ }\hat{ } | g/cm³ | g/cm³ |
| 19 | Natural | — | 0.18 | 2.70 | 0.75 |
| | CaCO₃ | | | | |
| 20 | BM-37 | 1* | 2.58 | 2.60 | 0.38 |
| 21 | C** | N.A. | 2.02 | 2.71 | 0.55 |
| 22 | AR-81 | 0.5 | 1.32 | 2.13 | 0.23 |
| 23 | AR-83 | 1 | 1.44 | 2.03 | 0.22 |
| 24 | AR-118 | 2 | 2.07 | 2.01 | 0.18 |
| 25 | AR-119 | 5*** | 5.25 | 1.93 | 0.19 |
| 26 | AR-135 | 1 | 1.44 | 2.01 | 0.24 |
| 27 | AR-120 | 2\hat{ } | 2.37 | 1.91 | 0.18 |

*The sample was taken from the batch mode of operation in - EXAMPLE 3.
**Commercial PCC - Aragonite; of Specialty Minerals Inc. (SMI); Opacarb ® A40.
***5 g of AR-119 were dissolved in a 10% HCl solution. No decanoic acid could be extracted or detected, as is expected of such molecules when they are subjected to heating for 8 hours at 500° C.
\hat{ }50 ppm of phosphoric acid were used in addition to the decanoic acid.
\hat{ }\hat{ }Aragonite is converted into calcite at T > 400° C.
^{†}Table 13 - the results of EXAMPLE 14 (A)hours at 500° C.
N.A.—Not available.

(D) Determination of the Specific Gravity (S.G.)—by a Gas Pycnometer

CaCO₃ powder (d₅₀=3.5 microns) of Polychrom, Israel—“Girulite-8” and AR-118, a product of the present invention, were tested in a AccPyc 1330 ex Micromeritics—USA. The results are given in Table 15a, as follows:

TABLE 15a
the results of EXAMPLE 14 (D):

| Sample | | S.G. | Average S.G. |
|---|---|---|---|
| Code | Test # | (g/cm³) | (g/cm³) |
| G-8 | 1 | 2.7544 | 2.7538 |
| | 2 | 2.7549 | |
| | 3 | 2.7547 | |
| | 4 | 2.7529 | |
| | 5 | 2.7523 | |
| AR-118 | 1 | 2.9070 | 2.8903* |
| | 2 | 2.8918 | |
| | 3 | 2.8932 | |
| | 4 | 2.8846 | |
| | 5 | 2.8750 | |

*This experiment demonstrates that: (i) the product of the present invention is permeable to air (especially under vacuum and pressure) and (ii) the product of the present invention would not have been discovered had this routine technique used to determine the S.G. of the products of the present invention and even those skilled in the art could overlook the whole phenomenon of “porous” PCC, which is the basis of this invention.

(5th) Final Determination of the Specific Gravity (S.G.) of a Product Calcined at 500° C.

In most practical instances the use of EXAMPLE 14(A) and EXAMPLE 14(C) to determine the specific gravity of the PCC products, may not cause an dispute, and a person of the art can observe quite easily that a product of the present invention is quite different from a prior art product, merely by observing the considerable differences between the apparent (loose) bulk density (L.B.D.) of the aragonite particles of the present invention, compared with those of proper art aragonite particles (Tables 13, 14 and 15). However, when the specific gravity (S.G.) of the PCC particles is quite close to 2.5 g/cm³, the accuracy of the analytical method may be of prime importance. In such cases especially, determination of the S.G. values should be conducted as follows:

(a) The S.G. should be determined according to (i) EXAMPLE 14(A) and EXAMPLE 14(C), and (ii) EXAMPLE 14(C) only (i.e. the dewatered sample of the product may be ignited at 500C without drying it first, or under the conditions customarily practiced in the prior art). These tests should be conducted three times and the determined average value will represent the final result in each case, (i) and (ii).

(b) The lowest of the S.G. results obtained for a particular product in (a) according to both (i) and (ii), independently, will determine whether the product (and the process) falls within the scope of the present invention (according to the embodiment where the S.G. is determinative).

(F) Determination of the Loose Bulk Density (L.B.D.) of a Product Dried at 120° C.

A sample, dried at 120° C. for twelve hours, was de-agglomerated gently using a mortar/pestle and sieved through a 0.6 mm screen. The L.B.D. of the fine powder that passed the screen was determined, separately and independently of the S.G analyses (c. f. EXAMPLE 14(E)) and the T.B.D. analyses (c. f. EXAMPLE 14(G)), according to the ASTM D1895. The average results, of three repetitions of the test, are reported already in Table 13 (above) and now in Table 15b as follows:

TABLE 15b
the results of EXAMPLE 14 (F)

| | | | | | L.B.D. | L.B.D.⁸ |
|---|---|---|---|---|---|---|
| | Sample | | CO₂ | Dosage | 120° C. | 500° C. |
| Test # | Code | Active agent | (%) | (wt. %) | (g/cm³) | (g/cm³) |
| 28 | Natural | — | — | — | 0.65 | 0.75 |
| | CaCO₃ | | | | | |
| 29 | GCC-8¹ | — | — | — | 0.65 | 0.56 |
| 30 | GCC-8² | Decanoic A. | N.A. | 2.0 | 0.64 | 0.56 |
| 31 | PCC³ | N.A. | N.A | N.A | 0.71 | 0.55 |
| 32 | PCC⁴ | Decanoic A. | N.A. | 2.0 | 0.70 | 0.53 |
| 33 | OM-95^{A} | — | — | — | 0.66 | 0.49 |
| 34 | UPCC^{B} | N.A. | N.A | N.A. | 0.50 | 0.37 |
| 35 | AR-81 | Decanoic A. | 100.0 | 0.5 | 0.31 | 0.23 |
| 36 | AR-83 | Decanoic A. | 100.0 | 1 | 0.30 | 0.22 |
| 37 | AR-118 | Decanoic A. | 100.0 | 2 | 0.25 | 0.18 |
| 38 | AR-119 | Decanoic A. | 100.0 | 5** | 0.29 | 0.19 |
| 39 | AR-135 | Decanoic A. | 100.0 | 1 | 0.31 | 0.24 |
| 40 | AR-120 | Decanoic A. | 100.0 | 2*** | 0.23 | 0.18 |
| 41 | ARP-35 | Decanoic A. | 26.0 | 1.5 | 0.33 | 0.18 |
| 42 | ARP-36 | Decanoic A. | 26.0 | 2.0 | 0.33 | 0.20 |
| 43 | ARP-61 | Decanoic A. | 100.0 | 2.0 | 0.38 | 0.25 |
| 44 | ARP-62-1 | Decanoic A. | 100.0 | 3.0 | 0.31 | 0.28 |
| 45 | ARP-51 | Lauric A. | 26.0 | 1.5 | 0.31 | 0.17 |
| 46 | ARP-65 | Lauric A. | 50.0 | 1.5 | 0.38 | 0.21 |
| 47 | ARP-76⁶ | Undecylenic A. | 50.0 | 1.5 | 0.38 | 0.18 |
| 48 | ARP-77 | Myristic A. | 50.0 | 1.5 | 0.37 | 0.18 |
| 49 | ARP-70⁷ | Stearic A. | 50.0 | 1.5 | 0.37 | 0.19 |
| 50 | ARP-71 | Isostearic A. | 50.0 | 1.5 | 0.69 | 0.53 |
| 51 | ARP-72 | Oleic A. | 50.0 | 1.5 | 0.92 | 0.53 |
| 52 | ARP-83 | Palmitic A. | 50.0 | 1.5 | 0.86 | 0.54 |

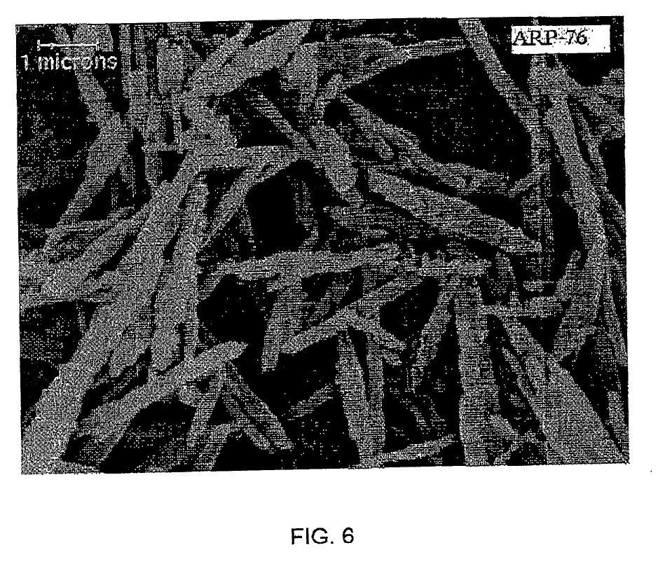

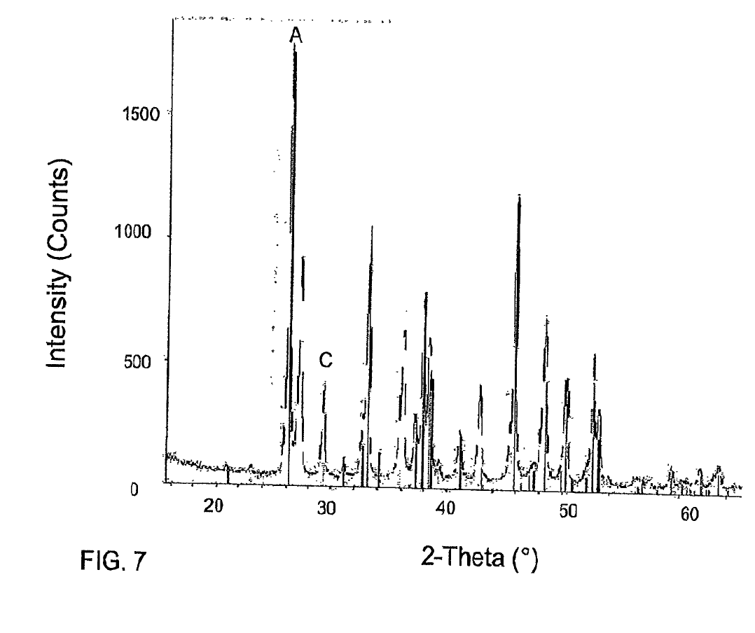

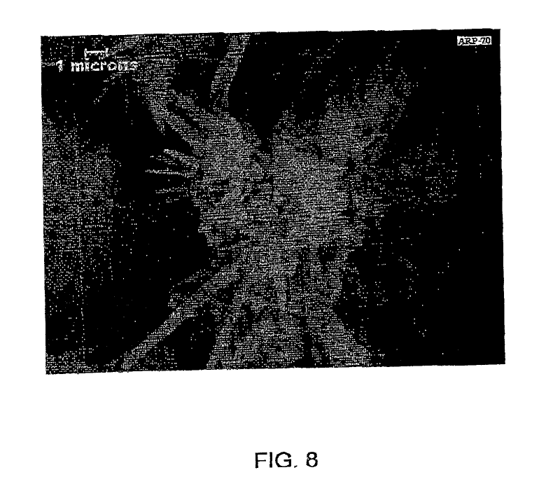

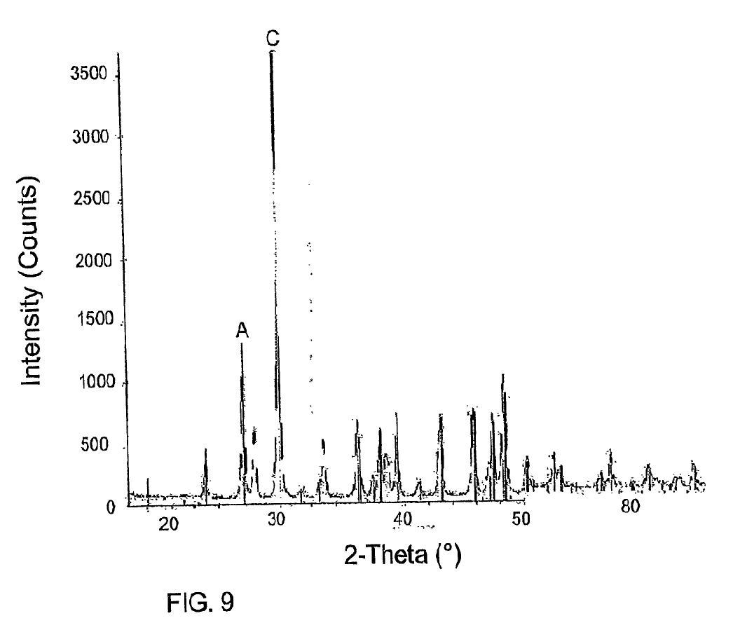

**5 g of AR-119 were dissolved in a 10% HCl solution. No decanoic acid could be extracted or detected, as is expected of such molecules when they are subjected to cheating for 8 hours at 500° C.
***50 ppm of phosphoric acid were used in addition to the decanoic acid.
¹“Girulite-8” CaCO3 powder (d50 = 3.5 microns) ex Polychrom - Israel.
²“Girulite-8” CaCO3 powder (d50 = 3.5 microns) ex Polychrom - Israel, which was coated using 2 wt. % n-decanoic acid.
³Commercial PCC - Aragonite (SSA = 12 m²/g); of Specialty Minerals Inc. (SMI); Opacarb ® A40.
⁴Commercial PCC - Aragonite; of Specialty Minerals Inc. (SMI); Opacarb ® A40, which was coated using 2 wt. % n-decanoic acid.
⁵50 ppm of phosphoric acid were used in addition to the decanoic acid.
⁶The XRD spectrum and SEM of ARP-76 are presented in FIG. 6 and FIG. 7, respectively.
⁷The XRD spectrum and SEM of ARP-70 are presented in FIG. 8 and FIG. 9, respectively.
⁸The L.B.D. of the calcined powder was determined in a similar manner.
^{A}Commercial ultrafine stearic acid coated - UFT 95 GCC natural calcite (95 wt. % pass 2μ size) ex Omya-Pluess-Staufer - Switzerland.
^{B}Commercial ultrafine (SSA = 19 m²/g) stearic acid coated - Ultraflex PCC calcite ex SMI - U.S.A.
N.A.—Not Available.

The results in Tables 13 and in Table 15b represent the products of the present invention if they have a L.B.D. <0.55 g/cm³. However, those results count, if the SSA (BET) of the specific samples in test are <15 m²/g and they are coated by the respective active agents that were used (in order to minimize the variations of surface interactions). Those samples that do not meet this requirement, can only be tested according to EXAMPLE 14(E).

Conclusion: the product (and process) in question will belong to the present invention, if it passes either this test (i.e. L.B.D. <0.55 g/cm³) or the T.B.D. test (i.e. T.B.D. <0.70 g/cc³). Should the product in question fail to pass both (T.B.D. & L.B.D.) tests, its S.G. values (according to EXAMPLE 14(E)) will determine if it is the product (the process) according to an embodiment of the present invention.

It may be noted that dramatic L.B.D. changes occur when the products of the present invention are subjected to high temperature treatment at 300° C. (c.f. Table 4) and especially at 500° C. (c. f. Table 15), which are probably due to the thermal collapse of the “porous” structure of these particles.

(G) Determination of the Tapped Bulk Density (T.B.D.) of a Product Dried at 120° C.

A dry sample (at 120° C. for twelve hours) was de-agglomerated gently using a mortar/pestle and sieved through a 0.6 mm screen. The T.B.D. of the fine powder that passed the screen was determined, separately and independently of the S.G. analyses (c. f. EXAMPLE 14(E)) and the L.B.D. analyses (c. f. EXAMPLE 14(F)) analyses. The fine powder is introduced into a 250 ml caliberated plastic graduate cylinder, which is then mounted on a screen-shaker (e. g. Rotap Model RX-29-10 ex W. S. Tyler Inc.—USA). The apparatus is then operated and the volume of the powder is inspected intermittently (e. g. after 5, 10, 20, 30 and 40 minutes) until no change is observed. The highest T.B.D. value is the final result of the test. This test is repeated three times for each sample and the reported T.B.D. being the average of these three tests. The results are as follows:

TABLE 15c
the results of EXAMPLE 14 (G)

| | | | | T.B.D. | T.B.D. | T.B.D. |
|---|---|---|---|---|---|---|
| | | | T.B.D. | 120° C. | 120° C. | 120° C. |
| | | L.B.D. | 120° C. | (g/cm³) | (g/cm³) | (g/cm³) |
| | Sample | 120° C. | (g/cm³) | 10 | 20 | 30 |
| # | Code* | (g/cm³) | 5 mins. | mins. | mins. | mins. |
| 53 | GCC-8¹ | 0.65 | 0.94 | 0.93 | 0.99 | 0.99 |
| 54 | GCC-8² | 0.64 | 1.09 | 1.17 | 1.27 | 1.27 |
| 55 | PCC³ | 0.71 | 0.76 | 0.78 | 0.82 | 0.82 |
| 56 | PCC⁴ | 0.70 | 0.86 | 0.86 | 0.97 | 0.97 |
| 57 | OM-95⁵ | 0.66 | 0.88 | 0.92 | 0.92 | 0.92 |
| 58 | UPCC⁶ | 0.50 | 0.64 | 0.65 | 0.66 | 0.66 |
| 59 | ARP-33 | 0.34 | 0.53 | 0.56 | 0.62 | 0.62 |
| 60 | ARP-35 | 0.33 | 0.49 | 0.57 | 0.63 | 0.63 |
| 61 | ARP-36 | 0.33 | 0.49 | 0.57 | 0.61 | 0.61 |
| 62 | ARP-51 | 0.31 | 0.42 | 0.44 | 0.45 | 0.45 |
| 63 | ARP-65 | 0.38 | 0.53 | 0.59 | 0.62 | 0.62 |
| 64 | ARP-70⁷ | 0.37 | 0.53 | 0.58 | 0.60 | 0.60 |
| 65 | ARP-76⁸ | 0.38 | 0.54 | 0.61 | 0.69 | 0.69 |
| 66 | ARP-77 | 0.37 | 0.45 | 0.52 | 0.59 | 0.59 |
| 67 | ARP-71 | 0.69 | 0.90 | 1.10 | 1.11 | 1.11⁹ |
| 68 | ARP-72 | 0.70 | 0.87 | 1.10 | 1.12 | 1.12⁹ |
| 69 | ARP-83 | 0.86 | 1.05 | 1.16 | 1.22 | 1.22⁹ |
| ¹“Girulite-8” CaCO3 powder (d50 = 3.5 microns) ex Polychrom - Israel. | | | | | | |
| ²“Girulite-8” CaCO3 powder (d50 = 3.5 microns) ex Polychrom - Israel, which was coated using 2 wt. % n-decanoic acid. “Girulite-8” CaCO3 powder (d50 = 3.5 microns) ex Polychrom - Israel, which was coated using 2 wt. % n-decanoic acid (the coating is conducted by adding slowly the carboxylic acid (e.g. decanic acid) into a well stirred aqueous slurry of the GCC at 90° C. The product is then dewatered and dried at 120° C. for twelve hours). | | | | | | |
| #It is worthwhile noting that the final T.B.D. value of this coated product is now 1.27 g/cm³ (>0.99 g/cm³ before coating¹). | | | | | | |
| ³Commercial PCC - Aragonite (SSA = 12 m²/g); of Specialty Minerals Inc. (SMI); Opacarb ® A40. | | | | | | |
| ⁴Commercial PCC - Aragonite; of Specialty Minerals Inc. (SMI); Opacarb ® A40, which was coated using 2 wt. % n-decanoic acid (the coating is conducted by adding slowly the carboxylic acid (e.g. decanic acid) into a well stirred aqueous slurry of the PCC at 90° C. The product is then dewatered and dried at 120° C. for twelve hours). It is worthwhile noting that the final T.B.D. value of this coated product is | | | | | | |
| #now 0.97 g/cm³ (0.82 g/cm³ before coating³). | | | | | | |
| ⁵A commercial ultrafine stearic acid coated - UFT 95 GCC natural calcite (95 wt. % pass 2μ size) ex Omya-Pluess-Staufer - Switzerland. | | | | | | |
| ⁶A commercial ultrafine (SSA = 19 m²/g) stearic acid coated - Ultraflex PCC calcite ex SMI - U.S.A. | | | | | | |
| ⁷The XRD spectrum and SEM of ARP-70 are presented in FIG. 8 and FIG. 9, respectively. | | | | | | |
| ⁸The XRD spectrum and SEM of ARP-76 are presented in FIG. 6 and FIG. 7, respectively. | | | | | | |
| ⁹It is worthwhile noting that the T.B.D. values of products that were produced according to all the | | | | | | |
| #parameters of the process of the present invention, except the active agents that were changed, are quite similar to those of the products of the prior art, and especially to coated calcite GCC². | | | | | | |

The results in Table 15c represent the products of an embodiment of the present invention if they have a T.B.D. <0.70 g/cm³. However, those results count, if the SSA (BET) of the specific samples in test are <15 m²/g and they are coated by the respective active agents that were used (in order to minimize the variations of surface interactions). Those samples that do not meet this requirement, can only be tested according to EXAMPLE 14(E).

Conclusion: the product (and process) in question will belong to the present invention, if it passes either this test (i.e. T.B.D. <0.70 g/cm³) or the L.B.D. test (i. e. L.B.D. <0.55 g/cc³). Should the product in question fail to pass both (T.B.D. & L.B.D.) tests, its S.G. values (according to EXAMPLE 14(E)) will determine if it is the product (the process) of an embodiment of the present invention.

(H) Determination of the Specific Gravity (S.G.) in Oils of Products Dried at 120° C.

The specific gravity of various calcium carbonate particles was measured in various oils, as described in Example 14(A). The results indicate that the low S.G. values are not due to the tall oil that was used, but it is rather common to many similar liquids. In cases at which the S.G. values are close to 2.5 g/cm³, the well-defined oleic acid (of purity >97%) can be used to resolve any dispute, and in any case, the instructions in EXAMPLE 14(A) and EXAMPLE 14(E), still prevail.

The Process Set Points—Continuous Mode of Operation:

1. Rotor Speed=2500 rpm (Rotor Diameter=8.5 cm)

2. pH=9.5±0.2

3. Temperature=85° C.±3

4. Carbon dioxide flow rate=2 m³/hr.

5. Aqueous calcium hydroxide slurry (of Shfeya) −10 wt. %=˜50-70 L.P.H. (to maintain the preset pH value).

6. Active agent concentration decanoic acid; 0-2 wt. % based on CaCO₃.

7. Reactor Volume=30 l. (Diameter=8.7 cm).

The results are as follows:

TABLE 15d
the results of EXAMPLE 14 (H):

| | | | | | | S.G.⁷ |
|---|---|---|---|---|---|---|
| | | | | | | g/cm³ |
| Test | Sample | Active | Dosage | CO2 | | 120 |
| # | Code | Agent | (wt %) | (%) | Liquid^{6,7} | ° C. |
| 70 | GCC-8¹ | — | — | — | Sylvatal 20S | 2.63 |
| 71 | PCC³ | — | — | N.A. | Sylvatal 20S | 2.56 |
| 72 | GCC-8² | Decanoic A. | 2.0 | — | Sylvatal 20S | 2.32 |
| 73 | PCC⁴ | Decanoic A. | 2.0 | N.A. | Sylvatal 20S | 2.31 |
| 74 | AR-118⁵ | Decanoic A. | 2.0 | 100.0 | Sylvatal 20S | 1.77 |
| 75 | AR-118⁵ | Decanoic A. | 2.0 | 100.0 | Sylvatal 20S | 1.75 |
| 76 | AR-118⁵ | Decanoic A. | 2.0 | 100.0 | Oleic (>97%) | 1.79 |
| 77 | ARP-29 | Decanoic A. | 0.7 | 26.0 | Sylvatal 20S | 1.98 |
| 78 | ARP-31 | Decanoic A. | 1.0 | 26.0 | Sylvatal 20S | 1.65 |
| 79 | ARP-35 | Decanoic A. | 1.5 | 26.0 | Sylvatal 20S | 1.57 |
| 80 | ARP-35 | Decanoic A. | 1.5 | 26.0 | Oleic (>97%) | 1.64 |
| 81 | ARP-35 | Decanoic A. | 1.5 | 26.0 | Oleic (>97%) | 1.62 |
| 82 | ARP-35 | Decanoic A. | 1.5 | 26.0 | Canola Oil | 1.66 |
| 83 | ARP-35 | Decanoic A. | 1.5 | 26.0 | Soybean Oil | 1.60 |
| 84 | ARP-35 | Decanoic A. | 1.5 | 26.0 | Sunflower Oil | 1.58 |
| 85 | ARP-35 | Decanoic A. | 1.5 | 26.0 | Corn Oil | 1.61 |
| 86 | ARP-35 | Decanoic A. | 1.5 | 26.0 | Mazola Oil | 1.59 |
| 87 | ARP-35 | Decanoic A. | 1.5 | 26.0 | Olive Oil | 1.63 |
| 88 | ARP-36 | Decanoic A. | 2.0 | 26.0 | Sylvatal 20S | 1.34 |
| 89 | ARP-36 | Decanoic A. | 2.0 | 26.0 | Sylvatal 20S | 1.35 |
| 90 | ARP-36 | Decanoic A. | 2.0 | 26.0 | Oleic (>97%) | 1.36 |
| 91 | ARP-37 | Decanoic A. | 2.0 | 15.0 | Sylvatal 20S | 1.28 |
| 92 | ARP-51 | Lauric A. | 1.5 | 26.0 | Sylvatal 20S | 1.78 |
| 93 | ARP-61 | Decanoic A. | 2.0 | 100.0 | — | — |
| 94 | ARP-62-1 | Decanoic A. | 3.0 | 100.0 | — | — |
| 95 | ARP-65 | Lauric A. | 1.5 | 50 | Sylvatal 20S | 2.04 |
| 96 | ARP-70 | Stearic A. | 1.5 | 50 | Sylvatal 20S | 2.36 |
| 97 | ARP-76 | Undecylenic | 1.5 | 50 | Sylvatal 20S | 2.02 |
| | | A. | | | | |
| 98 | ARP-77 | Myristic A. | 1.5 | 50 | Sylvatal 20S | 2.15 |
| 99 | ARP-71 | Isostearic A. | 1.5 | 50 | Sylvatal 20S | 2.71 |
| 100 | ARP-72 | Oleic A. | 1.5 | 50 | Sylvatal 20S | 2.58 |
| 101 | ARP-78 | Linoleic A. | 1.5 | 50 | Sylvatal 20S | 2.59 |
| 102 | ARP-83 | Palmitic A. | 1.5 | 50 | Sylvatal 20S | 2.61 |
| ¹“Girulite-8” CaCO₃ powder (d50 = 3.5 microns) ex Polychrom - Israel. | | | | | | |
| ²“Girulite-8” CaCO₃ powder (d50 = 3.5 microns) ex Polychrom - Israel, which was coated using 2 wt. % n-decanoic acid (the coating is conducted by adding slowly the carboxylic acid (e.g.decanic acid) into a well stirred aqueous slurry of the GCC at 90° C. The product is then dewatered and dried at 120° C. for twelve hours). It is worthwhile noting that the S.G. value of this coated | | | | | | |
| #product is now < 2.5 g/cm³ (>2.5 g/cm³ before coating¹). However, this low S.G. value does not at all indicate | | | | | | |
| #that this product after its coating according to a prior art procedure belongs now to the present invention. Its use under the real down-stream conditions does not produce a significant effect, as is revealed in the experimental section. For instance, the hiding powder results obtained with this coated/uncoated product under experimental conditions of Example 19(A) were so low that they were not presented at all in Table 34. | | | | | | |
| ³Commercial PCC - Aragonite; of Specialty Minerals Inc. (SMI); Opacarb ® A40. | | | | | | |
| ⁴Commercial PCC - Aragonite; of Specialty Minerals Inc. (SMI); Opacarb ® A40, which was coated using 2 wt. % n-decanoic acid (the coating is conducted by adding slowly the carboxylic acid (e.g. decanic acid) into a well stirred aqueous slurry of the PCC at 90° C. The product is then dewatered and dried at 120° C. for twelve hours). It is worthwhile noting that the S.G. value of this coated product is | | | | | | |
| #now < 2.5 g/cm³ (>2.5 g/cm³ before coating³). However, this low S.G. value does not | | | | | | |
| #at all indicate that this product after its coating according to a prior art procedure belongs now to the present invention. Its use under the real down-stream conditions does not produce a significant effect, as is revealed in the experimental section. For instance, the hiding powder results obtained with this coated/uncoated product under experimental conditions of Example 19(A) are presented in Table 34. | | | | | | |
| ⁵AR-118, a product of the present invention, mentioned already in EXAMPLE 14 (A), above. | | | | | | |
| ⁶The specific gravity of the liquids were as follows: Oleic A. = 0.887 g/cm³; Tall Oil (Sylvatal 20S ex Arizona Chemical - USA) = 0.930 g/cm³; Refined Canola Oil (ex Milumor - Israel) = 0.910 g/cm³; Refined Corn Oil (ex Milumor - Israel) = 0.910 g/cm³; Refined Corn Oil (Mazola ex Bestfoods - USA) = 0.915 g/cm³; Refined Soybean Oil (ex | | | | | | |
| #Milumor - Israel) = 0.920 g/cm³; Refined Sunflower Oil (ex Milumor - Israel) = 0.918 g/cm³; Cold Pressed olive Oil (ex Shemen Industries - Israel) = 0.893 g/cm³; | | | | | | |

⁷The S.G. analyses were conducted at 21° C. ± 1° C.
N.A.—Not available

### EXAMPLE 15

### Preparation of Exterior White Paint—Hercules Inc

The procedure for the preparation of this paint was obtained from Hercules Inc.; Cellulose & Protein Products D.; Wilmington, Del. 19899 (USA). The procedure followed quite closely the Celanese Resins Formulation No. EP-48-222 for the production of this Exterior White Paint (Vinyl Acetate & Acrylate).

(A) The Ingredients Used for the 52% PVC Paint and their Function are as Follows:

1. Tap water.

2. Nopco NDW defoamer.

3. Cellosize QP 15000 thickener (hydroxy ethyl cellulose).

4. Disperse One (45% N.V.) (dispersant).

5. Synperonic NP10 surfact; wetting agent.

6. Kronos 2160 TiO₂ pigment.

7. Synthetic sodium aluminum silicate (p820) (spacer).

8. Kaolin clay (D₅₀=3.1 micron)(spacer).

9. CaCO₃ (spacer)—A GCC product of Polichrom Ltd., Israel.

10. Talc (D₅₀=12.3 micron)(spacer).

11. PCC—Aragonite of the present invention (samples used contained >80% CaCO₃ in the wet cake products before their drying; no diminution operation took place prior to this use. Namely, the PCC—Aragonite used is not necessarily yet optimized for its purpose).

12. Copolymer vinyl acetate acrylate (55% N.V.) (emulsion).

13. Butyl diglycol acetate solvent (coalescent agent).

14. Kathon LXE preservative.

15. 25% Ammonia (base).

16. Tap water.

Tap water (1), defoamer (2) and thickener (3) were added to a plastic container (d=20 cm; h=30 cm) equipped with a disk (d=8 cm) attached to a Dissolver (Homo Dispers Model HD-550 (0.75 HP) of Hsiangtai Machinery Industry Co. Ltd.; Taiwan). The mixture was stirred at 500 rpm for 5 minutes, after which the dispersant (4) and the wetting agent (5) were added, and stirring was continued at 500 rpm for additional 5 minutes. At this point the stirring speed was increased to 1500 rpm and the respective ingredients for the respective formulations 1-10 were added consecutively, each ingredient over a 5 minute period, according to the order in the above list of reagents (6-16).

The physical properties of the above paints were measured, including the most important property—the hiding power (%) of 90 μm layers of paint were determined with an ACS instrument (Applied Color Systems) and the results are given in the following Tables 19 and 20:

TABLE 19
the results of EXAMPLE 15
Exterior White Paint - Hercules Inc.
Evaluation of the 52% PVC Paints Based on the Precipitated
Aragonite Calcium Carbonate Particles of the Present Invention
(PCC - Aragonite)

| | 1 | 2 | 3 | 4 | 5 | 6 |
|---|---|---|---|---|---|---|
| Raw Material | % (wt) | | | | | |
| Tap Water | 25.0 | 25.0 | 25.0 | 25.0 | 25.0 | 25.0 |
| Defoamer | 0.1 | 0.1 | 0.1 | 0.1 | 0.1 | 0.1 |
| Thickener | 0.3 | 0.3 | 0.3 | 0.3 | 0.3 | 0.3 |
| (15K) | | | | | | |
| Dispersant | 0.9 | 0.9 | 0.9 | 0.9 | 0.9 | 0.9 |
| (45%) | | | | | | |
| Wetting Agent | 0.35 | 0.35 | 0.35 | 0.35 | 0.35 | 0.35 |
| TiO₂; Kronos | 14.0 | 9.0 | 9.0 | 9.0 | 9.0 | 8.4 |
| Silicate | 3.7 | 3.7 | 3.7 | 3.7 | 4.0 | 3.9 |
| CaCO₃ - GCC | 7.0 | — | — | — | — | — |
| Kaolin Clay | 6.5 | 6.5 | 6.5 | 6.5 | 10.7 | 6.8 |
| Talc | 6.3 | — | — | — | — | — |
| PCC- | — | 17.75 | 17.75 | 17.75 | 13.00 | 17.75 |
| Aragonite* | | | | | | |
| Copolymer | 24.5 | 25.5 | 25.5 | 25.5 | 25.4 | 25.4 |
| (55%) | | | | | | |
| Coalescent | 0.1 | 0.1 | 0.1 | 0.1 | 0.1 | 0.1 |
| Agent | | | | | | |
| Preservative | 0.5 | 0.5 | 0.5 | 0.5 | 0.5 | 0.5 |
| Ammonia | 0.3 | 0.3 | 0.3 | 0.3 | 0.3 | 0.3 |
| Tap Water | 10.45 | 10.0 | 10.0 | 10.0 | 10.35 | 10.2 |
| Total | 100 | 100 | 100 | 100 | 100 | 100 |

The Characteristics of the Paint

| | | | | | | |
|---|---|---|---|---|---|---|
| Solids (%) | 50.98 | 50.98 | 50.98 | 50.98 | 50.67 | 50.82 |
| P.V.C. (%) | 51.77 | 51.32 | 51.32 | 51.32 | 51.51 | 51.55 |
| Hiding Power | 94.0 | 94.4 | 94.9 | 95.5 | 95.1 | 94.9 |
| (%) | | | | | | |
| Viscosity | 92.0 | 92.0 | 93.2 | 98.0 | 100.0 | 98.0 |
| (K.U.) | | | | | | |
| Hegman | 4.5 | 5.5 | 5.5 | 5.0 | 4.0 | 4.5 |
| Bulk Density | 1.317 | 1.259 | 1.257 | 1.248 | 1.226 | 1.221 |
| (g/cm³) | | | | | | |
| Saving of TiO₂ | — | 35.7 | 35.7 | 35.7 | 35.7 | 40.0 |
| (%) | | | | | | |
| Weight Saving | — | 4.4 | 4.6 | 5.2 | 6.9 | 7.3 |
| (%) | | | | | | |

| Formulation | | Sample | | Active Agent |
|---|---|---|---|---|
| No. | Pigment* | Code | Active Agent | % (wt) |
| 1 | Reference Paint | — | — | — |
| 2 | PCC - Aragonite | AR-81 | Decanoic acid | 0.5 |
| 3 | PCC - Aragonite | AR-83 | Decanoic acid | 1.0 |
| 4 | PCC - Aragonite | AR-118 | Decanoic acid | 2.0 |
| 5 | PCC - Aragonite | AR-119 | Decanoic acid | 5.0 |
| 6 | PCC - Aragonite | AR-118 | Decanoic acid | 2.0 |

TABLE 20
the results of EXAMPLE 15
Exterior White Paint - Hercules Inc.
Evaluation of the 52% PVC Paints Based on the Precipitated
Aragonite Calcium Carbonate Particles of the Present Invention
(PCC - Aragonite)

| | 1 | 7 | 8 | 9 | 10 |
|---|---|---|---|---|---|
| Raw Material | % (wt) | | | | |
| Tap Water | 25.0 | 25.0 | 25.0 | 25.0 | 25.0 |
| Defoamer | 0.1 | 0.1 | 0.1 | 0.1 | 0.1 |
| Thickener (15K) | 0.3 | 0.3 | 0.3 | 0.3 | 0.3 |
| Dispersant (45%) | 0.9 | 0.9 | 0.9 | 0.9 | 0.9 |
| Wetting Agent | 0.35 | 0.35 | 0.35 | 0.35 | 0.35 |
| TiO₂; Kronos | 14.0 | 7.0 | 7.0 | 6.3 | 12.0 |
| Silicate | 3.7 | 4.8 | 4.2 | 4.2 | 3.7 |
| CaCO₃ - GCC | 7.0 | — | — | — | — |
| Kaolin Clay | 6.5 | 7.1 | 12.5 | 13.1 | 6.5 |
| Talc | 6.3 | — | — | — | — |
| PCC-Aragonite* | — | 17.75 | 13.0 | 13.0 | 15.3* |
| Copolymer (55%) | 24.5 | 25.5 | 26.0 | 26.0 | 24.5 |
| Coalescent Agent | 0.1 | 0.1 | 0.1 | 0.1 | 0.1 |
| Preservative | 0.5 | 0.5 | 0.5 | 0.5 | 0.5 |
| Ammonia | 0.3 | 0.3 | 0.3 | 0.3 | 0.3 |
| Tap Water | 10.45 | 10.3 | 9.75 | 9.85 | 10.45 |
| Total | 100 | 100 | 100 | 100 | 100 |

The Characteristics of the Paint

| | | | | | |
|---|---|---|---|---|---|
| Solids (%) | 50.98 | 50.98 | 50.98 | 50.98 | 50.98 |
| P.V.C. (%) | 51.77 | 51.32 | 51.32 | 51.32 | 51.91 |
| Hiding Power (%) | 94.0 | 94.2 | 94.3 | 94.0 | 92.7 |
| Viscosity (K.U.) | 92.0 | 96.8 | 98.8 | 98.0 | 90.2 |
| Hegman | 4.5 | 4.5 | 4.5 | 4.5 | 4.5 |
| Bulk Density (g/cm³) | 1.317 | 1.179 | 1.159 | 1.224 | 1.300 |
| Saving of TiO₂ (%) | — | 50.0 | 50.0 | 55.0 | 14.3 |
| Weight Saving (%) | — | 10.5 | 12.0 | 7.0 | 1.3 |

| Formulation | | Sample | | Active Agent |
|---|---|---|---|---|
| No. | Pigment* | Code | Active Agent | % (wt) |
| 1 | Reference Paint | — | — | — |
| 7 | PCC - Aragonite | AR-118 | Decanoic acid | 2.0 |
| 8 | PCC - Aragonite | AR-119 | Decanoic acid | 5.0 |
| 9 | PCC - Aragonite | AR-119 | Decanoic acid | 5.0 |
| 10 | PCC - Aragonite | C** | N.A. | N.A. |

**Commercial PCC - Aragonite; of Specialty Minerals Inc. (SMI); Opacarb ® A40.

(B) The ingredients of the 32% PVC Paint and their function are as follows

1. Tap water.

2. Nopco NDW defoamer.

3. Cellosize QP 15000 thickener (hydroxy ethyl cellulose).

4. Disperse One (45% N.V.) (dispersant).

5. Synperonic NP10 surfactant; wetting agent.

6. Kronos 2160 TiO₂ pigment. 7.

7. Synthetic Na—Al silicate (p820) (spacer).

8. Kaolin clay (D₅₀=3.1 micron)(spacer)

9. CaCO₃ (spacer)—a GCC product of Polichrom Ltd., Israel

10. Talc (D₅₀=12.3 micron)(spacer)

11. PCC—aragonite of the present invention (samples used contained >80% CaCO₃ in the wet cake products before their drying; no diminution operation took place prior to this use. However, the PCC—aragonite used is not necessarily yet optimized for its purpose).

12. Propylene glycol (solvent).

13. Copolymer vinyl acetate acrylate (55% N.V.) (emulsion).

14. Butyl diglycol acetate solvent (coalescent agent).

15. Kathon LXE preservative.

16. 25% Ammonia (base).

17. Tap water.

Tap water (1), defoamer (2) and thickener (3) were added to a plastic container (d=20 cm; h=30 cm) equipped with a disk (d=8 cm) attached to a Dissolver (Homo Dispers Model HD-550 (0.75 HP) of Hsiangtai Machinery Industry Co. Ltd.; Taiwan). The mixture was stirred at 500 rpm for 5 minutes, after which the dispersant (4) and the wetting agent (5) were added, and stirring was continued at 500 rpm for additional 5 minutes. At this point the stirring speed was increased to 1500 rpm and the respective ingredients for the respective formulations 1-10 were added consecutively, each ingredient over a 5 minute period, according to the order in the above list of reagents (6-15).

The physical properties of the above paints were measured, including the most important property—the hiding power (%) of 90 μm layers of paint were determined with an ACS instrument (Applied Color Systems) and the results are given in the following Table 21:

TABLE 21
the results of EXAMPLE 15
Exterior White Paint - Hercules Inc.
Evaluation of the 32% PVC Paints Based on the Precipitated Aragonite
Calcium Carbonate Particles of the Present Invention (PCC - Aragonite)

| | 11 | 12 | 13 | 14 | 15 | 16 |
|---|---|---|---|---|---|---|
| Raw Material | % (wt) | | | | | |
| Tap Water | 17.8 | 17.8 | 17.8 | 17.8 | 17.8 | 17.8 |
| Defoamer | 0.15 | 0.15 | 0.15 | 0.15 | 0.15 | 0.15 |
| Thickener | 0.22 | 0.22 | 0.22 | 0.22 | 0.22 | 0.22 |
| (15K) | | | | | | |
| Dispersant | 0.60 | 0.60 | 0.60 | 0.60 | 0.60 | 0.60 |
| (45%) | | | | | | |
| Wetting Agent | 0.34 | 0.34 | 0.34 | 0.34 | 0.34 | 0.34 |
| TiO₂; Kronos | 22.5 | 20.0 | 20.0 | 19.0 | 18.0 | 19.0 |
| Silicate | 2.25 | 2.25 | 2.25 | 2.25 | 2.25 | — |
| CaCO3 - GCC | 5.0 | — | — | — | — | — |
| PCC- | — | 7.5 | 7.5 | 8.5 | 9.25 | 13.0 |
| Aragonite* | | | | | | |
| Propylene | 2.70 | 2.70 | 2.70 | 2.70 | 2.70 | 2.70 |
| Glycol | | | | | | |
| Copolymer | 37.45 | 37.45 | 37.45 | 37.45 | 38.0 | 33.4 |
| (55%) | | | | | | |
| Coalescent | 0.18 | 0.18 | 0.18 | 0.18 | 0.18 | 0.18 |
| Agent | | | | | | |
| Preservative | 0.45 | 0.45 | 0.45 | 0.45 | 0.45 | 0.45 |
| Ammonia | 0.30 | 0.30 | 0.30 | 0.30 | 0.30 | 0.30 |
| Tap Water | 10.06 | 10.06 | 10.06 | 10.06 | 9.76 | 11.86 |
| Total | 100. | 100. | 100. | 100. | 100. | 100. |

The Characteristics of the Paint

| | | | | | | |
|---|---|---|---|---|---|---|
| Solids (%) | 50.35 | 50.35 | 50.35 | 50.35 | 50.35 | 50.37 |
| P.V.C. (%) | 32.59 | 32.64 | 32.64 | 32.93 | 32.64 | 36.73 |
| Hiding | 91.0 | 91.4 | 91.4 | 91.0 | 90.8 | 91.0 |
| Power (%) | | | | | | |
| Viscosity | 87.2 | 98.0 | 97.4 | 96.2 | 87.8 | 90.2 |
| (K.U.) | | | | | | |
| Hegman | 5.5 | 5.5 | 5.5 | 5.5 | 5.0 | 5.5 |
| Bulk Density | 1.18 | 1.128 | 1.127 | 1.109 | 1.005 | 1.073 |
| (g/cm³) | | | | | | |
| Saving of | — | 11.1 | 11.1 | 15.5 | 20.0 | 15.5 |
| TiO₂ (%) | | | | | | |
| Weight | — | 4.4 | 4.5 | 6.0 | 14.8 | 6.8 |
| Saving (%) | | | | | | |

| | | | | Active |
|---|---|---|---|---|
| Formulation | | Sample | Active | Agent |
| No. | Pigment* | Code | Agent | % (wt) |
| 11 | Reference Paint | — | — | — |
| 12 | PCC - Aragonite | AR-118 | Decanoic acid | 2.0 |
| 13 | PCC - Aragonite | AR-119 | Decanoic acid | 5.0 |
| 14 | PCC - Aragonite | AR-119 | Decanoic acid | 5.0 |
| 15 | PCC - Aragonite | AR-119 | Decanoic acid | 5.0 |
| 16 | PCC - Aragonite | AR-118 | Decanoic acid | 2.0 |

Notes:

1. The particulate precipitated aragonite calcium carbonate of the present invention (PCC-Aragonite) can be used to produce paints without a substantial prior size reduction, except that effected by the mixing system of the production of the paint, which is anyway being used in this art to thoroughly disperse the pigments in the various formulations.

2. Though the particulate precipitated aragonite calcium carbonate of the present invention (PCC-Aragonite) is not yet optimized for its use in the production of paints and though the formulations used are by no means optimized, still this product is able to substitute over 50% of the expensive titanium oxide pigment without any deterioration of the resulting paint, as it manifested by the hiding power measured.

3. As the coatings (paints) are being sold and used by volume, and not by weight, the additional saving resulting from using the particulate precipitated aragonite calcium carbonate of the present invention (PCC-Aragonite) can surpass 10% on all the constituents of the coating, including the titanium oxide.

4. For simplicity in formulating the above mentioned paints, dry samples of The particulate precipitated aragonite calcium carbonate of the present invention (PCC-Aragonite), were used. However, wet filter cakes that contain even more water than 20% wt. %, based on wet CaCO₃ cake, can be used, provided that this water is being taken in account. However, on an industrial scale, dry PCC-Aragonite will be rarely used, due to the economy of using the wet product.

(C) A Comparison of Modified Paint Formulations Containing Various GCC/PCC

The experimental of EXAMPLE 15(A) was repeated, except that the paint compositions contained only one selected PCC/GCC pigment (>50 wt %) at a time and the minimum required ingredients that were necessary to prepare these basic modified paint formulations. A standard (STD) interior paint formulation was used as a general reference.

The paint compositions are as follows:

TABLE 22
STD vs. Modified paint formulation of EXAMPLE 15 (C)

| Raw Material | STD | Modified |
|---|---|---|
| Water | 28.25 | 28.87 |
| Defoamer | 0.30 | 0.38 |
| Thickener | 0.20 | 0.09 |
| Dispersant | 0.40 | 0.00 |
| Wetting Agent | 0.30 | 0.00 |
| TiO₂ | 7.5 | 0.00 |
| Silicate | 3.5 | 0.00 |
| Talc | 13.0 | 0.00 |
| G.C.C. | 34.0 | 0.00 |
| AR-pigment | 0.00 | 57.28 |
| Propylene glycol | 1.00 | 0.56 |
| Copolymer - 50% solids | 11.40 | 12.72 |
| Biocide | 0.15 | 0.10 |
| Total | 100.0 | 100.0 |
| % Solids in paint | 63.7 | 64.3 |

The physical and optical properties of the pigments used and the paint obtained are reported as follows:

TABLE 23
the pigments properties in EXAMPLE 15 (C)

| | | | | | S.S.A. | | |
|---|---|---|---|---|---|---|---|
| | Pigment | Active | Dosage | CO₂ | (BET) | PSD⁴ μ | |
| Paint | Code | Agent | (wt %)³ | (v %) | (m²/g) | D₉₀ | D₅₀ |
| STD | — | — | — | — | — | — | — |
| 1 | PCC¹ | — | — | — | 12.0 | 2.0 | 0.8 |
| 2 | GCC² | — | — | — | 4.0 | 5.0 | 2.1 |
| 3 | ARP-29 | Decanoic A. | 0.7 | 26.0 | 5.7 | 4.49 | 1.98 |
| 4 | ARP-31 | Decanoic A. | 1.0 | 26.0 | 6.2 | 3.54 | 1.68 |
| 5 | ARP-34 | Decanoic A. | 1.5 | 100 | 11.1 | 2.68 | 1.27 |
| 6 | ARP-35 | Decanoic A. | 1.5 | 26.0 | 11.5 | 3.0 | 1.65 |
| 7 | ARP-36 | Decanoic A. | 2.0 | 26.0 | 15.4 | 2.51 | 0.73 |
| 8 | ARP-37 | Decanoic A. | 2.0 | 15.0 | 10.9 | 2.20 | 0.93 |
| 9 | ARP-51 | Lauric A. | 1.5 | 26.0 | | 4.14 | 2.39 |
| 10 | ARP-61 | Decanoic A. | 2.0 | 100 | 15.5 | 1.8 | 0.68 |
| 11 | ARP-65 | Lauric A. | 1.5 | 50.0 | 8.2 | 5.4 | 3.1 |
| 12 | ARP-70 | Stearic A. | 1.5 | 50.0 | 4.3 | 5.3 | 1.6 |
| 13 | ARP-71 | Isostearic | 1.5 | 50.0 | 1.9 | 9.3 | 5.9 |
| 14 | ARP-72 | Oleic A. | 1.5 | 50.0 | 3.7 | 10.9 | 6.5 |
| 15 | ARP-76 | Undecylenic | 1.5 | 50.0 | 13.1 | 2.7 | 1.5 |
| | | A | | | | | |
| 16 | ARP-77 | Myristic A. | 1.5 | 50.0 | 5.2 | 3.8 | 2.3 |
| 17 | ARP-83 | Palmitic A. | 1.5 | 50.0 | | | |

¹Commercial stable PCC Slurry of Aragonite (Opacarb ® A40 ex SMI - USA). Calculation of the S.G. of the aragonite particles in a 69.6% commercial slurry resulted in 2.83 g/cm³. Naturally, this slurry was used to produce the paint above.
²CaCO3 (spacer) - a GCC (Calcite ex Polichrom Ltd. - Israel).
³Calculated as the acid form, based on the CaCO3.
⁴The PCC of the present invention has not undergone any size reduction prior to its use, except the size reduction that may happen during regular operations.

TABLE 24
the paint properties in EXAMPLE 15 (C)

| | | | Dosage | | S.G.⁴ | Paint | |
|---|---|---|---|---|---|---|---|
| Paint | | Active | (wt | | 120° C. | | Hiding⁶ |
| Code | Pigment Code | Agent | %)³ | CO₂ (v %) | (g/m³) | Gloss⁵ | Power |
| STD | — | — | — | — | — | 2.1 | 83.7 |
| 1 | PCC¹ | — | — | — | 2.56 | 31.9 | 88.0 |
| 2 | GCC² | — | — | — | 2.63 | 2.2 | 80.9 |
| 3 | ARP-29 | Decanoic | 0.7 | 26.0 | 1.98 | 3.0 | 92.6 |
| | | A. | | | | | |
| 4 | ARP-31 | Decanoic | 1.0 | 26.0 | 1.65 | 3.6 | 96.5 |
| | | A. | | | | | |
| 5 | ARP-34 | Decanoic | 1.5 | 100 | — | 7.5 | 96.3 |
| | | A. | | | | | |
| 6 | ARP-35 | Decanoic | 2.0 | 26.0 | 1.57 | 4.0 | 99.6 |
| | | A. | | | | | |
| 7 | ARP-36 | Decanoic | 2.0 | 26.0 | 1.34 | 5.5 | 98.1 |
| | | A. | | | | | |
| 8 | ARP-37 | Decanoic | 2.0 | 15.0 | 1.28 | 5.2 | 99.5 |
| | | A. | | | | | |
| 9 | ARP-51 | Lauric | 1.5 | 26.0 | 1.78 | 4.3 | 94.8 |
| | | A. | | | | | |
| 10 | ARP-61 | Decanoic | 2.0 | 100 | — | 11.5 | 98.3 |
| | | A. | | | | | |
| 11 | ARP-65 | Lauric | 1.5 | 50.0 | 2.04 | 4.0 | 93.7 |
| | | A. | | | | | |
| 12 | ARP-70 | Stearic | 1.5 | 50.0 | 2.36 | 2.6 | 94.1 |
| | | A. | | | | | |
| 13 | ARP-71 | Isostearic | 1.5 | 50.0 | 2.71 | 1.8 | 70.0 |
| 14 | ARP-72 | Oleic | 1.5 | 50.0 | 2.58 | 1.9 | 74.9 |
| | | A. | | | | | |
| 15 | ARP-76 | Undecylenic | 1.5 | 50.0 | 2.02 | 4.7 | 96.8 |
| | | A.⁷ | | | | | |
| 16 | ARP-77 | Myristic | 1.5 | 50.0 | 2.15 | 3.5 | 95.1 |
| | | A. | | | | | |
| 17 | ARP-83 | Palmitic | 1.4 | 50.0 | 2.61 | 2.0 | 79.3 |
| | | A. | | | | | |
| ¹Commercial PCC a stable slurry of Aragonite (Opacarb ® A40 ex SMI - USA). | | | | | | | |
| ²CaCO3 (spacer) - a GCC (Calcite ex Polichrom Ltd. - Israel). | | | | | | | |
| ³Calculated as the acid form, based on the CaCO3. | | | | | | | |
| ⁴Measured according to Example 14 (A), after drying at 120° C. for twelve hours (c.f. Tables 13 and 15d). | | | | | | | |
| ⁵Measured at 60° with a Glossmeter (Minigloss 101 N ex Sheen Instruments - England). | | | | | | | |
| ⁶The hiding power of a 90 μ layer was measured with a reflectometer (Ref. 310 Sheen-Opac ex Sheen Instruments - England). | | | | | | | |
| ⁷This compound, for example, can be brominated and thus serves also as a flame retardant. | | | | | | | |
| Notes: | | | | | | | |
| 1. The gloss increases as the v % of the CO₂ in the feed gas increases. The gloss increases as the wt. % of the active agent increases. The gloss increases as the specific gravity (S.G.) of the PCC decreases. The gloss increases as the specific gravity (S.G.) of the PCC decreases. These facts are particularly important in controlling the PCC of the present invention, and more specifically, in formulating high-gloss paper coatings on the one hand and it is particularly | | | | | | | |
| # important in formulating low-gloss paints on the other hand. | | | | | | | |

2. The opacity increases as the wt % of the active agent increases. The opacity increases as the V % of the air in the feed gas increases. The opacity increases as the specific gravity (S.G.) of the PCC decreases. n-Decanoic acid seems to exhibit, thus far, the best performance, however, the optimal w % seems to be in the range between 1.5 wt % to 3 wt % for this purpose of forming products of high hiding power.

### EXAMPLE 16

### The Preparation of the Plastic (Polypropylene Copolymer—PP) Formulations

The composition of the various formulations was as follows: 40% Filler, 0.3% antioxidant (irganox B225 ex Ciba Specialty Chemicals—Switzerland), 0.5% lubricant (Wax PE 520 ex Hoechst-Celanese—USA) and 59.2% polypropylene copolymer (Capilene-TR50 ex Carmel Olefins—Israel).

(A) Preparation of the Parficillate Precipitated Aragonite

Three samples of particulate precipitated aragonite of the present invention were used and three of the top quality commercial samples were used for comparison. The preparation parameters of the aragonite samples and their properties are given in Table 25, as follows:

The Process Set Points—Continuous Mode of Operation:

1. Rotor Speed=4000 rpm (Tip Speed˜10 m/sec.)

2. pH=9.5.

3. Temperature=90° C.

4. Carbon dioxide flow rate=180 L.P.H. (liters/hour).

5. Aqueous calcium hydroxide slurry (of Shfeya) −10 wt. %=˜6 L.P.H. (to maintain the preset pH value).

6. Active agent concentration=decanoic acid; 0.5; 1; 2 wt. % based on CaCO₃.

TABLE 25
the results of EXAMPLE 16(A)

| | | | | Aragonite | | | SSA |
|---|---|---|---|---|---|---|---|
| | Sample | | | Aragonite + Calcite | | B** | (BET) |
| # | Code | Active agent | (wt. %) | (1st)XRD | D₉₀¹ μ | (%) | m²/g |
| 1 | AR-213 | Decanoic Acid | 0.5* | 93-96% | 5.92 | 95.8 | 3.6 |
| 2 | AR-214 | Decanoic Acid | 1.0* | 93-96% | 4.38 | 98.3 | 6.4 |
| 3 | AR-215 | Decanoic Acid | 2.0* | 96-98% | 1.56 | 98.2 | 12.8 |

*50 ppm of phosphoric acid were used in addition to the decanoic acid to increase the aspect ratio of the acicular aragonite.
**Brightness.
¹The PCC of the present invention has not undergone any size reduction prior to its use, except the size reduction that may happen during regular operations.

(B) Compounding of the Plastic (Polypropylene Copolymer) Formulation.

The formulations were processed in a co-rotating twin-screw compounder (L/D=24 ex Dr. Collin - Germany). The compounding conditions are given in Table 26, as follows:

TABLE 26
the compounding conditions of EXAMPLE 16(B)

| | | Melt | Screws | | |
|---|---|---|---|---|---|
| | | Temp. | Speed | Pressure | Torque |
| # | Filler | (° C.) | (rpm) | (bar) | (N.m) |
| 1 | AR-215 | 203 | 200 | 16 ± 5 | 42 ± 5 |
| 2 | AR-214 | 203 | 200 | 15 ± 5 | 40 ± 2 |
| 3 | AR-213 | 202 | 200 | 18 ± 5 | 43 ± 2 |
| 4 | OM-95^{A} | 201 | 175 | 30 ± 5 | 63 ± 2 |
| 5 | UPCC^{B} | 203 | 174 | 30 ± 5 | 60 ± 3 |
| 6 | UTALC^{C} | 202 | 195 | 17 ± 2 | 49 ± 2 |

^{A}Commercial ultrafine stearic acid coated - UFT 95 GCC natural calcite (95 wt % pass 2 μ size) ex Omya-Pluess-Staufer - Switzerland.
^{B}Commercial ultrafine stearic acid coated - Ultraflex PCC calcite ex SMI - USA.
^{C}Commercial ultrafine talc - Ultratalc 609 ex SMI - USA.
Note:
The results of the compounding step, in Table 16b, indicate that the PCC of the present invention is superior over the commercial products of the top qualities and top prices in the market.

(C) Injection of the Plastic (Polypropylene Copolymer) Formulations

The resulting granules were fed to an injection machine (25 t ex Dr. Boy—Germany). Specimens of 127×12.7×3.2 mm we produced. The injection conditions are given in Table 27 as follows:

TABLE 27
the compounding conditions of EXAMPLE 16c

| | | | Injection | Injection | Injection |
|---|---|---|---|---|---|
| | | Temp. | Speed | Pressure | Pressure |
| # | Filler | (° C.) | (cm³/sec.) | (bar) | (bar) |
| 1 | AR-215 | 170-180 | 66 | 250 | 250 |
| 2 | AR-214 | 170-180 | 66 | 250 | 250 |
| 3 | AR-213 | 170-180 | 66 | 250 | 300 |
| 4 | OM-95^{A} | 170-180 | 66 | 250 | 300 |
| 5 | UPCC^{B} | 170-180 | 66 | 400 | 400 |
| 6 | UTALC^{C} | 170-180 | 66 | 400 | 400 |

^{A}a commercial ultrafine stearic acid coated GCC ex Omya-Pluess-Staufer - Switzerland.
^{B}a commercial ultrafine stearic acid coated - Ultraflex PCC ex SMI - USA.
^{C}a commercial ultrafine talc - Ultratalc 609 ex SMI - USA.
Note:
Only OM-95 behaves quite close to the PCC of the present invention (AR-213, Ar-214 and AR-215) in the injection compartment. The relatively (very) expensive talc is unable to compete with AR-213, Ar-214 and AR-215.

(D) The Mechanical Tests

The resulting specimens were conditioned at 25° C. under 50% RH for at least 72 hrs. before testing them.

Two test were performed as follows:

Flexure testing (3 point) was conducted according to ASTM D-790.

Impact testing—Izod notched was conducted according to ASTM D-256.

The results are given in table 28, as follows:

TABLE 28
the results of the mechanical tests of EXAMPLE 16d

| | | Flex. | |
|---|---|---|---|
| | | Modulus | Impact |
| # | Filler | (Mpa) | (J/m) |
| 0 | —* | 793 | 538 |
| 1 | AR-215 | 1603 ± 60 | 225 ± 21.7 |
| 2 | AR-214 | 1872 ± 90 | 281 ± 18.7 |
| 3 | AR-213 | 1735 ± 31 | 397 ± 14.5 |
| 4 | OM-95^{A} | 1107 ± 20 | 192 ± 20.4 |
| 5 | UPCC^{B} | 1006 ± 13 | 51 ± 6.4 |
| 6 | UTALC^{C} | 1749 ± 54 | 151 ± 5.3 |
| *Capilene - TR50 as reported by its producer. | | | |
| ^{A}a commercial ultrafine stearic acid coated GCC ex Omya-Pluess-Staufer - Switzerland. | | | |
| ^{B}a commercial ultrafine stearic acid coated - Ultraflex PCC ex SMI - USA. | | | |
| ^{C}a commercial ultrafine talc - Ultratalc 609 ex SMI - USA. | | | |
| Notes: | | | |
| ¹Fillers are usually added to the polypropylene (PP) formulations to increase their flexural modulus. In the proper loading range, the higher the concentration of the filler, the higher is the flexural modulus. However, as the concentration of the filler increases, the Izod impact characteristics are decreased dramatically. Namely, the final loading of the filler in the polymer is the result of optimizing both characteristics of the final (consumer) products. Under the same experimental | | | |
| # conditions, the particulate precipitated calcium carbonate of the present invention (AR-213, Ar-214 and AR-215) are by far superior over commercial products of the highest quality in the market. | | | |

²The overall properties of the PCC of the present invention are superior over the commercial products of top qualities in the market, as it may leads to faster operations and to better (consumer) products.

### EXAMPLE 17

### Adsorption Experiments Using the PCC of the Present Invention

The PCC/GCC particles of the prior art can adsorb limited quantities of liquids and in all cases that will take place quite fast onto their surface.

The PCC of the present invention exhibits a varied behavior, depending on he environment at which these particles are located.

(A) The PCC. Particles of the Present Invention in the Gas Phase

The results that were obtained by using the gas pycnometer to determine the specific gravity (S.G.) of the product of the present invention (c.f. EXAMPLE 14(D)) do not indicate at all that these particles contain some kind of “pores” or “bubbles” or “blisters” to any extend. However, the following results will demonstrate that this view is entirely wrong.

(B) The PCC Particles of the Present Invention in Stable Aqueous Dispersions

The Process Set Points—Continuous Mode of Operation:

1. Rotor Speed=1200 rpm (Rotor Diameter 10 cm)

2. pH 9.5±0.2

3. Temperature 85° C.±2

4. Carbon dioxide flow rate=2.5 m³/hr.

5. Aqueous calcium hydroxide slurry (of Shfeya) −10 wt. %=˜80-100 L.P.H. (to maintain the preset pH value).

6. CO₂ (v %) in the feed gas=30%

7. Active agent concentration=decanoic acid; 1.5 wt. % based on CaCO₃.

8. Reactor Volume=50 1. (Diameter=30 cm).

9. The product (of the present invention)—AR-P-73

Preparation of Stable Slurries (60-70 wt %) in Water

The wet cake of ARP-73, which was obtained after dewatering of the product, was mixed with about 2 wt % dispersant (resulting in calculated ˜1:1 dry weight ratio; Dispex N-40 (˜45 wt % solids) ex Allied Colloids—GB) and water) in a 10 l. (d=30 cm) tank that was equipped with laboratory dissolver (DVH-020-18/6; 2.5 HP ex a Dantco Inc.—USA; rotor diameter=10 cm) for 60 mins. at 1200 rpm. The resulting slurries exhibit the following characteristics:

TABLE 29
the results of EXAMPLE 17 (B)

| | | S.G. of Slurry | | |
|---|---|---|---|---|
| | Solids | (g/cm³)⁴ | | S.G. of Solids |
| Sample | In Slurry | After | After | ARP-73 |
| Code | (wt %)¹ | Preparation¹ | 7 Days² | (g/cm³)¹ |
| ARP-73-1 | 60.2 | 1.47 | 1.47 | 2.13 |
| ARP-73-2 | 69.3 | 1.61 | 1.61 | 2.20 |
| ARP-73-3 | 63.0 | 1.55 | 1.55 | 2.30 |
| | | | 1.68³ | >2.79³ |
| ¹Was measured and/or immediately after the preparation of the slurry, but no efforts were made to minimize the S.G. values of the PCC. | | | | |
| ²Was measured and calculated, but no efforts were made to minimize the S.G. values of the PCC. | | | | |
| ³The addition of 2 wt % sodium dioctylsulfosuccinate (75% in ethanol; ex Cytec - USA) - a potent wetting agent - to the water at the end of the preparation stage (+ a short stirring thereafter), led to the formation of a very dense and viscose mass after 7 days (most of the water have been absorbed by the PCC of the present invention!), | | | | |
| #which could not be stirred. This mass was disintegrated by adding more of the wetting agent and stirring the mass. The S.G. of the slurry was then measured³ (1.68 g/cm³) and the calculated³ S.G. of the PCC was >2.79 g/cm³. Such behavior is unprecedented in the prior art of PCC/GCC products. | | | | |

⁴The low S.G. values are also observed in e.g. the paint compositions containing the products of the present invention, which gives rise to an additional advantage, as was explained already in EXAMPLE 15.

(C) Dispersions of the PCC Particles of the Present Invention in Organic Solvents

Attempts to obtain stable slurries of the PCC of the present invention in organic solvents such as alcohols (e.g. methanol, ethanol, isopropanol), ketones (e.g. acetone, methyl ethyl ketone), esters (e.g. methyl acetate, ethyl acetate), aromatic solvents (e.g. toluene, xylene, chlorobenzene, o-dichlorobenzene), and many others, result in the formation of mixtures in which the specific gravity (S.G.) of the PCC particles is >2.5 g/cm³ (usually, water and oils (e.g. those that are mentioned as liquids in Examples 14(A) and 14(H)), permeate very slowly during very long periods and in some cases it is impractical to measure their permeation rates). More polar solvents such as ethylene glycol penetrate more slowly into the PCC of the present invention, and eventually the S.G. values reach the ultimate value that characterizes calcite, and especially aragonite, calcium carbonate (namely, ˜2.7-2.9 g/cm³, depending on the specific crystallographic purity of the tested products). Naturally, the increase of the S.G. is dependent on many factors such as pressure, temperature, viscosity, surface tension, purity, and naturally the quality of the PCC product of the present invention.

It is worthwhile noting that the unique properties of the original PCC particles of the present invention are fully restored once the organic solvent is evaporated to dryness, and this product can be used in the same application or another one as the original sample. More specifically, after the removal of e.g. acetone, toluene, or ethyl acetate, all the properties of a used sample e. g. of AR-120, matched the original sample.

The phenomenon described in this EXAMPLE 17 leads to the conclusion that the PCC of the present invention can readily be used as an adsorbent for liquids (solvent), as a carrier (encapsulant) for liquids and solids (by dissolving them in a suitable solvent; allowing the solution to penetrate into the “pores” of the PCC; and removing the solvent by e.g. evaporation or dissolution of the solvent in another solvent that reduces the solubility of the substrate. The PCC of the present invention can encapsulate many compounds, including e.g. pharmaceuticals (medicines), agrochemicals, flavors, fragrances and sunscreen agents (this PCC itself is particularly suitable for protecting the human skin, once its particles are fine-tuned for that purpose. Due to the trapped gas (air) in the PCC of the present invention, this task of fine-tuning the PSD to meet the requirements to protect from incoming light at 300-400 mμ, is expected to be much less expensive than of e. g. the TiO₂, which is used for this purpose quite often). Therefore, the PCC of the present invention offers two functions in one material, namely, encapsulation and efficient light dispersion). Moreover, the “porous” nature of this PCC makes it a preferable candidate to serve as a filler, a builder and/or an anticaking agent in e. g. powder detergents, etc.

To summarize, the PCC of the present invention can serve in any capacity that calcium carbonate particles of the prior art serve, and additionally it possesses many advantages due to its “porous” nature.

The results of mixing experiments at which a sovent only or a solution is used to form a thick and dense viscose mass are reported as follows:

TABLE 30
the results of EXAMPLE 17 (C)

| | PCC/GCC¹ | Liquid | | | Residual |
|---|---|---|---|---|---|
| # | Product | (Solvent) | Substrate-(wt %) | Ratio wt/wt² | Liquid² |
| 1 | ARP-73 | Water | DOSSNa-2 | 1.0 | − |
| 2 | “Girulite-8” | Methanol | — | 1.2 | + |
| 3 | Opacarb ® A40 | Methanol | — | 1.2 | + |
| 4 | ARP-35 | Methanol | — | 1.2 | − |
| 5 | ARP-37 | Methanol | — | 1.2 | − |
| 6 | ARP-76 | Methanol | — | 1.2 | − |
| 7 | “Girulite-8” | Methanol³ | p-Anisaldehyde-5 | 1.2 | + |
| 8 | Opacarb ® A40 | Methanol³ | p-Anisaldehyde-5 | 1.2 | + |
| 9 | ARP-35 | Methanol^{3,4} | p-Anisaldehyde-5 | 1.2 | − |
| 10 | ARP-37 | Methanol^{3,4} | p-Anisaldehyde-5 | 1.2 | − |
| 11 | ARP-76 | Methanol^{3,4} | p-Anisaldehyde-5 | 1.2 | − |
| 12 | “Girulite-8” | Acetone³ | HBCD-5 | 1.2 | + |
| 13 | Opacarb ® A40 | Acetone³ | HBCD-5 | 1.2 | + |
| 14 | ARP-35 | Acetone^{3,5} | HBCD-5 | 1.2 | − |
| 15 | ARP-37 | Acetone^{3,5} | HBCD-5 | 1.2 | − |
| 16 | ARP-76 | Acetone^{3,5} | HBCD-5 | 1.2 | − |
| 17 | “Girulite-8” | Ethyl Acetate³ | Caffeine-0.5 | 1.2 | + |
| 18 | Opacarb ® A40 | Ethyl Acetate³ | Caffeine-0.5 | 1.2 | + |
| 19 | ARP-35 | Ethyl Acetate^{3,6} | Caffeine-0.5 | 1.2 | − |
| 20 | ARP-37 | Ethyl Acetate^{3,6} | Caffeine-0.5 | 1.2 | − |
| 21 | ARP-76 | Ethyl Acetate^{3,6} | Caffeine-0.5 | 1.2 | − |
| 17 | “Girulite-8” | Acetonitrile³ | Pd (II) Diacetate-2 | 1.2 | + |
| 18 | Opacarb ® A40 | Acetonitrile³ | Pd (II) Diacetate-2 | 1.2 | + |
| 19 | ARP-35 | Acetonitrile^{3,7} | Pd (II) Diacetate-2 | 1.2 | — |
| 20 | ARP-37 | Acetonitrile^{3,7} | Pd (II) Diacetate-2 | 1.2 | — |
| 21 | ARP-76 | Acetonitrile^{3,7} | Pd (II) Diacetate-2 | 1.2 | — |
| ¹The respective powder was mixed thoroughly with the liquid (solvent; solution) in a tightly closed 100 cm³ erlenmeyer and allowed to stand for one hour at 21° C. ± 1° C. Test #1 lasted longer, as water permeates much slower - described in EXAMPLE 17 (B). | | | | | |
| ²The weight ratio of liquid/powder (which was de-agglomerated and sieves through a 0.6 mm screen. The fines that passed the screen were used). | | | | | |
| ³At the end of each test, the presence of a liquid layer on top of the slurry and its flowability was marked by a plus sign, while the absence of such a layer and the formation of a thick viscose mass was marked by a minus sign. | | | | | |
| ⁴The products of the present invention were then dried, de-agglomerated and sieved through a 0.6 mm screen. The fines that passed the screen were stored for stability tests. This experiment was repeated using the following substrates: p-Anisaldehyde (a flavor & fragrance); 2-Ethylhexyl trans-Cinnamate (a sunscreen agent); (-) Menthon (a pharmaceutical & fragrance); Menthol (a flavor & fragrance); Anise Alcohol (ex Koffolk - Israel) (a flavor & fragrance); | | | | | |
| # Methyl Raspberry Ketone (a flavor & fragrance); and Lilial (a fragrance). | | | | | |

⁵The products of the present invention were then dried, de-agglomerated and sieved through a 0.6 mm screen. The fines that passed the screen were stored for stability tests. This experiment was repeated using the following substrates: Hexabromocyclododecane (Syntex HBCD ™ ex Albemarle - U.S.A.) (a flame retardant for plastics); and NeendX ™ex Albemarle - U.S.A. (a non-halogen flame retardant for plastics); Diazinon (Diazol ex Makhteshim-Agan - Israel) (an insecticide).
⁶The products of the present invention were then dried, de-agglomerated and sieved through a 0.6 mm screen. The fines that passed the screen were stored for stability tests. This experiment was repeated using the following substrates: Caffeine (a pharmaceutical); and (-) Menthon (a pharmaceutical and fragrance).
⁷The products of the present invention were then dried, de-agglomerated and sieved through a 0.6 mm screen. The fines that passed the screen were stored for for further calcination and hydrogenation (other precious metals could be used in a similar manner).
Notes:
1. The “porous” product of the present invention may absorb considerable quantities of solvents (>50% of its weight).
2. The existence of “pores” in the PCC of the present invention has been established by indirect evidence, However, it is not of much consequence if we are still not too accurate in depicting the exact properties that are responsible for the effects that were encountered.

### EXAMPLE 18

### Preparation of Ultra-Lightweight Coated (ULWC) Paper

(A) The Pigments

Using methods that are known to the paper industry, a series of PCC pigments of the present invention (AR), with variations in particle size and distribution, was compared to commercial pigments that are customarily being used in the prior art. The AR products were used, as obtained in their production process, for comparison with Opacarb A40 (a commercial product ex SMI—USA). Characterization data is as follows:

TABLE 31
characterization of the pigments of EXAMPLE 18:

| Pigment | AR-110B | AR-F1 | AR-245-S | A40 |
|---|---|---|---|---|
| Identification | 4449-90.3 | 4449-90.4 | 3911-17 | 4327-48 |
| PH | — | 7.7 | 9.6 | 10.3 |
| % solids | 72.7 | 71.8 | 72.9 | 70.8 |
| Brook. 20 | 15150 | 10400 | 8040 | 220 |
| Visc. 50 | 6800 | 4480 | 3832 | 111 |
| 100 | 3700 | 3480 | 2204 | 110 |
| Hercules | 290 | ND | 441 | 1440 |
| PSD¹ @ 90 | 2.25 | 1.83 | 2.52 | 1.76 |
| 50 | 0.81 | 0.69 | 1.69 | 0.43 |
| 20 | 0.41 | 0.35 | 1.40 | 0.27 |
| 10 | 0.29 | 0.24 | 1.32 | 0.21 |
| SSA¹ | 7.5 | 6.8 | 6.8 | 12.2 |
| SD(90/20)² | 2.34 | 2.29 | 1.34 | 2.55 |
| Dry Rd | 95.0 | 94.3 | 95.3 | 95.9 |
| a | −0.6 | −0.5 | −0.6 | −0.1 |
| b | 0.3 | 0.8 | 1.0 | 0.8 |

¹It is worthwhile noting that the particles of the AR series are much larger than those of A40, which is also being reflected in the SSA values, respectively.
²SD (90/20) is used instead of GSD (84/16) to reflect the particle size distribution because d84 and d16 values were not available for the experimental pigments.

The clay control formulation developed with CPI consisted of 85 parts Kaowhite delaminated clay, 5 parts Ansilex 93 calcined clay and 10 parts TiO₂. Carbonates were used at 33 parts replacing an equal amount of delaminated clay. Binders and additives included Styronal 4606 SX latex and PG290 starch at 9 parts each and 0.7 parts Nopcote C-104 calcium stearate. Solids were adjusted to 60%. All formulations were coated at 2500 fpm on CPI groundwood stock (28#) to bracket the target of 3.5#/R. Coated sheets were calendered to achieve a gloss of 40 for the lowest weight clay control sample. Conditions were 2 nips at 600 pli and 150° F.

(B) Pigment and Coating Color

The paste-like consistency of the AR-F1 sample made determination of Hercules rheology of this pigment impossible. The high Brookfield viscosities of the AR series is most likely due to their unique and unusual thickening ability. A check of adequate dispersants and dispersant levels on these pigments is not presented herein, because of the proprietary nature of the dispersant package. The results are as follows:

TABLE 32
coating color results of EXAMPLE 18:

| Formu- | | | | | |
|---|---|---|---|---|---|
| lation | 4576-10-1 | 4576-10-2 | 4576-10-8 | 4576-10-9 | 4576-10-10 |

| | | | | | |
|---|---|---|---|---|---|
| Pigment | Clay | Opacarb | 4449-90.3 | 4449-90.4 | 3911-17 |
| | Control | A40 | AR-110B | AR-F1 | AR-245S |
| PH | 8.5 | 8.5 | 8.5 | 8.5 | 8.5 |
| % solids | 60.8 | 60.6 | 60.4 | 60.4 | 60.7 |
| Brook. 10 | 5680 | 5100 | 5100 | 5220 | 4800 |
| Visc. 20 | 3600 | 3100 | 3000 | 3150 | 2860 |
| 50 | 1940 | 1700 | 1528 | 1680 | 1484 |
| 100 | 1320 | 1000 | 950 | 1050 | 882 |
| Hercules | 50.0 | 50.7 | 36.1 | 40.3 | 50.0 |
| Rd | 81.6 | 83.6 | 83.6 | 83.5 | 84 |
| L | 90.4 | 91.4 | 91.4 | 91.4 | 91.7 |
| A | −1.7 | −1.8 | −1.7 | −1.7 | −1.7 |
| B | 4.7 | 3.9 | 3.8 | 3.9 | 3.6 |

Note:
Although AR-F1 displayed very high pigment Brookfield viscosity, no problems were observed when it was formulated into a coating.
Additionally, improvements in Hercules viscosity were observed with this pigment as well as with AR-110B after make down.
Hercules viscosity of AF-245S was equivalent to that of the control formulation.

(C) Coated Sheet Results

All data are interpolated to a value of 3.5#/R using linear regression of properties as a function of pigment coating weight. The results are as follows:

TABLE 33
coated sheet results interpolated to 3.5 #/R of EXAMPLE 18:

| Formu- | | | | | |
|---|---|---|---|---|---|
| lation | 4576-10-1 | 4576-10-2 | 4576-10-8 | 4576-10-9 | 4576-10-10 |
| | Clay | Opacarb | 4449-90.3 | 4449-90.4 | 3911-17 |
| Pigment | Control | A40 | AR-110B | AR-F1 | AR-245S |
| Brightness | 70.9 | 72.7 | 72.9 | 73.1 | 73.2 |
| Hunter L | 87.9 | 88.5 | 88.6 | 88.8 | 88.6 |
| a | −0.22 | −0.26 | −0.19 | −0.19 | −0.25 |
| b | 6.56 | 6.20 | 6.12 | 5.92 | 6.03 |

Notes:
1. Base on the results obtained and considering the facts: i. that the AR-products have been used as obtained in their production process, ii. that their particles were consideably larger (and their SSA values were considerably smaller) than the control samples, and iii. that the AR products and processes are not yet optimized for the purpose of making paper formulations, the AR products offer excellent pigments for the paper industry.
2. The brightness of the AR-coated papers is at least as good as that of the OPACARB A40 PCC sample, but is definitely better than other controls.

### EXAMPLE 19

### Coatings Made with Single Pigments—Comparison of Hiding Power (H.P.) Values

The H.P. of coatings that are made with single commercial pigments are compared. The pigments in this experiment include top quality commercial titanium dioxide pigments, top quality commercial CaCO₃ pigments and a precipitated particulate CaCO₃ of the present invention. As the coatings in this Example and the H. P. measurements are done under similar conditions, The differences among the various H.P. values reflect, mainly, the differences among the refractive indices of respective pigments (the Lorentz-Lorentz expression of M=[(nₚ/nₒ)²−1]/[(nₚ/nₒ)²+2]; where n, is the refractive index of the respective pigment and nₒ is the refractive index of the medium in which the respective pigments are immersed, is probably one of the best ways to correlate the H.P. of coatings—c.f. Pigment Handbook (Vol. I-III; Edited by T. C. Patton; John Wiley & Sons, New York (1973); Vol. III; Pages 289-290. A graphic illustration of the linear relation H.P. vs M² is given in FIG. 2 on Page 290).

Accordingly, the H.P. values of two coatings, in EXAMPLE 19, that contain top quality TiO₂ pigments are expected to be much higher than any of those coatings that are made with CaCO₃ pigment, only (for TiO₂ (n=2.76—Rutile; in Vol. I; Page 3 of the above Handbook) >> for CaCO₃ (n=1.530, 1.681 and 1.685—Orthorombic Aragonite; in Vol. I; Page 119 of the above Handbook)≅for CaCO₃ (n=1.486, 1.658; Calcite; in Vol. I; Page 119 of the above Handbook)).

It is worth noting that the coatings that are prepared below were formulated for one purpose only—to allow a proper comparison of the effective refractive indices of various pigments, including that of the CaCO₃ of the present invention. These coatings are not at all optimized to serve in the paint industry, but they should serve their purpose of creating a single matrix (with a single nₒ) to all the pigments in test.

(A) The Coating Formulation

Note: The % (wt) that are mentioned below relate to the final weight of the coating formulation, before it is being coated onto the hiding power chart.

To prepare ˜500 g coating in a 1 lit. beaker (d=12 cm), water (up to 100% (wt) of the final formulation), 0.2% (wt) thickener (Cellosize QP 15000 (hydroxy ethyl cellulose); a product of Union Carabide), 0.3% (wt) wetting agent (Nopco NDW; a product of Henkel) and 0.5% dispersant (Dispex N-40; a product of Allied Colloids) are added and the mixer (a dissolver; Hsiangtal; Model HD-550; equipped with saw-blade type rotor of d=9 cm) is operated at 400 rpm until a gel is formed.

55% (wt) of the pigment is then added to the beaker and the mixture is stirred at 1500 rpm for 20 minutes. The mixture is then tested with Hegmann (Sheens apparatus for fine grinding measurement gauge ref 501/100) until >+4 value is reached. The stirrer speed is then lowered to 400 rpm and 20% (wt) resin (Acronal 290D; a product of BASF that contains 50% (wt) acrylic-styrenic resin in water of which its dry form has a refractive index of about 1.5 which is a typical value (1.45-1.55) of many other resins that are being used in the coating industry) is added. The stirrer is operated for 20 minutes and the viscosity of the mixture is measured with a Stormer (Sheen 480). If necessary, about 0.1% (wt) thickener (TT 615 of Akzo) is added to bring the viscosity to 20 poise. Thereby, a coating formulation that contains 55% (wt) pigment is ready for use.

In order to lower the pigment concentation to preset values below 55% (wt), while still maintainig the pigment/resin ratio and the viscosity of the final coating formulation constant, the stirrer is operated at 400 rpm, the proper amount of water is added into the above formulation and about 0.1% (wt) thickener (TT 615; a product of Akzo) is added to bring the viscosity to 20 poise (this amount of the thickener is negligable and does not effect much the pigment concentration in the final coating).

The coating formulation is then mounted (thickness=90 micrometer) on the hiding power chart (Ref 301/2A ex Sheen instruments Ltd.); the coated paper is dried at ambient temperature for 24 hours and then in an oven at 40° C. until no change of its weight is observed.

The coated and dried paper is then subjected to a H.P. measurement using the 310 Sheen-Opac Reflectometer ex Sheen Instruments Ltd.

Each test is repreated three (3) times and the average value is presented in the table 34 and in graph 10, as follows:

(B) The Results

TABLE 34
Hiding Power Values of Coating Formulations
Series 1/Test #

| | S1 104-3 | 104-3-1 | 104-3-2 | 104-3-3 | 104-3-4 | 104-3-5 |
|---|---|---|---|---|---|---|
| Pigment Type | AR-66F - 2% Decanoic A. | | | | | |
| Quantity (g) | | 250 | 285 | 344.7 | 432.8 | 240.8 |
| TT 615 (g) | | 1.7 | 2 | 5.1 | 7 | 12 |
| H₂O Added (g) | | 31.2 | 63.3 | 98.48 | 173.1 | 160.5 |
| Solids (% wt) | 67.3 | 54.8 | 46.3 | 35.6 | 26.1 | 16.6 |
| Pigment (% wt) | 55.86 | 45.48 | 38.68 | 29.55 | 21.66 | 13.78 |
| H.P. | 99 | 95.8 | 91.6 | 85.8 | 75 | 50 |

Series 2/Test #

| | S1 104-5 | 104-5-1 | 104-5-2 | 104-5-3 | 104-5-4 | 104-5-5 |
|---|---|---|---|---|---|---|
| Pigment Type | TiO₂ - Kr 2160 | | | | | |
| Quantity (g) | | 214 | 233.2 | 218 | 219 | 179 |
| TT 615 (g) | | 6 | 6 | 4.3 | 4.8 | 4.8 |
| H₂O Added (g) | | 72.8 | 99.9 | 163 | 73 | 89.5 |
| Solids (% wt) | 67.4 | 48.8 | 34.2 | 18.7 | 15 | 9.8 |
| Pigment (% wt) | 55.94 | 40.50 | 28.39 | 15.52 | 12.45 | 8.13 |
| H.P. | 100 | 95.6 | 87 | 65.8 | 53.4 | 38 |

Series 3/Test #

| | S1 104-24 | 104-24-1 | 104-24-2 | 104-24-3 | 104-24-4 | 104-24-5 |
|---|---|---|---|---|---|---|
| Pigment Type | TiO₂ - Ti Pure R-706 | | | | | |
| Quantity (g) | | 242.9 | 290 | 308.1 | 286.5 | 210.3 |
| TT 615 (g) | | 1 | 1 | 1.2 | 1.2 | 1.3 |
| H₂O Added (g) | | 48.5 | 29 | 132 | 114.6 | 140.2 |
| Solids (% wt) | 67.4 | 55.4 | 51.5 | 35.3 | 23.7 | 13.6 |
| Pigment (% wt) | 55.94 | 45.98 | 42.75 | 29.30 | 19.67 | 11.29 |
| H.P. | 95.3 | 90.6 | 89.3 | 78.2 | 62.3 | 32.4 |

Series 4/Test #

| | S1 104-28 | Sl 104-28-1 | Sl 104-28-2 | Sl 104-28-3 | Sl 104-28-4 | Sl 104-28-5 |
|---|---|---|---|---|---|---|
| Pigment Type | Opacarb A40, Uncoated | | | | | |
| Quantity (g) | | 197.7 | 245.3 | 273.4 | 190.9 | 267.4 |
| TT 615 (g) | | 1 | 1.2 | 1.4 | 1.7 | 2.3 |
| H₂O Added (g) | | 39.54 | 24.93 | 117.17 | 76.36 | 178.26 |
| Solids (% wt) | 67.56 | 53 | 47.97 | 33.33 | 24.38 | 13.8 |
| Pigment (% wt) | 56.07 | 43.99 | 39.82 | 27.66 | 20.24 | 11.45 |
| H.P. | 84.1 | 77.1 | 73.8 | 59.7 | 47.2 | 26.4 |

Series 5/Test #

| | S1 104-25 | Sl 104-25-1 | Sl 104-25-2 | Sl 104-25-3 | Sl 104-25-4 | Sl 104-25-5 |
|---|---|---|---|---|---|---|
| Pigment Type | Opacarb A40, Coated with 2% Decanoic Acid | | | | | |
| Quantity (g) | | 225.8 | 273 | 251 | 260.9 | 238.7 |
| TT 615 (g) | | 1 | 1 | 1.3 | 3.6 | 4.1 |
| H₂O Added (g) | | 45.2 | 27.3 | 107.6 | 104.4 | 159 |
| Solids (% wt) | 68 | 54.8 | 49.2 | 34.2 | 24.7 | 16.2 |
| Pigment (% wt) | 56.44 | 45.48 | 40.84 | 28.39 | 20.50 | 13.45 |
| H.P. | 87 | 77.1 | 74 | 62.3 | 49.4 | 24.1 |

Series 6/Test #

| | S1 104-10 | 104-10-1 | 104-10-2 | 104-10-3 | 104-10-4 | 104-10-5 |
|---|---|---|---|---|---|---|
| Pigment Type | UFT 95 Omya | | | | | |
| Quantity (g) | | 206.3 | 235 | 255.6 | 205.7 | 177.1 |
| TT 615 (g) | | 1 | 1 | 1 | 1.3 | 1.2 |
| H₂O Added (g) | | 45.39 | 23.5 | 109.54 | 82.28 | 118.07 |
| Solids (% wt) | 67.1 | 54.6 | 48.9 | 32.3 | 23.5 | 14.4 |
| Pigment (% wt) | | 45.32 | 40.59 | 26.81 | 19.51 | 11.95 |
| H.P. | | 59.6 | 55.3 | 24.6 | 10.8 | 7 |

Series 7/Test #

| | S1 104-9 | 104-9-1 | 104-9-2 | 104-9-3 | 104-9-4 | 104-9-5 |
|---|---|---|---|---|---|---|
| Pigment Type | Ultrapflex UP | | | | | |
| Quantity (g) | | 175.7 | 177.3 | 193.2 | 270.7 | 195.9 |
| TT 615 (g) | | 1 | 1 | 1.2 | 1.2 | 1.3 |
| H₂O Added (g) | | 12.7 | 17 | 82.8 | 108.3 | 130.6 |
| Solids (% wt) | 59 | 54.9 | 48.8 | 31.4 | 32 | 21 |
| Pigment (% wt) | | 45.57 | 40.50 | 26.06 | 26.56 | 17.43 |
| H.P. | | 46 | 34.8 | 25.2 | 11.6 | 9.9 |

Series 1 - AR - 66F - 2% Decanoic A.; PCC Aragonite produced according to the present invention in a similar manner that is described in EXAMPLE 14A, but using a 50% (V) CO₂ gas. The product was used without any grinding and/or sieving. Dried poducts (e. g. at 120° C. for 12 hours) as well as aqueous slurries could be used, provided that the water content was taken in account.
Series 2 - TiO₂ - Kr 2160; a product of of Kronos.
Series 3 - TiO₂ - Ti Pure R-706; a product of Du Pont (organic treated).
Series 4 - Opacarb A40, Uncoated; a top quality PPC Aragonite product of SMI.
Series 5 - Opacarb A40, Coated with 2% Decanoic Acid; a PPC Aragonite product of SMI (controlled by Minerals Technologies Inc.) that was coated in our laboratory for comparison.
Series 6 - UFT 95 Omya; a top quality GCC Calcite product of Omya that is coated with 1-2% (wt) Stearic Acid.
Series 7 - Ultra-pflex UP; a top quality PCC Calcite product of SMI that is coated with 1-2% (wt) Stearic Acid.
Notes:
1. The H.P. values of the PCC of the present invention (Series 1) are quite comparable to those of the commertial TiO₂ pigments of Kronos (Series 2), but exceed those of the commertial TiO₂ pigments of Du Pont (Series 3), which is unexpected if only the well know refractive index of aragonite PCC is considered.
2. The H.P. values of the PCC of the present invention (Series 1) exceed those of all the top quality commertial CaCO₃ pigments (Series 4-7). That includes the results obtained for OPACARB A40 that was coated with 2% (wt) decanoic acid, which is unexpected if only the well know refractive index of aragonite PCC is considered.
3. The H.P. values of the PCC of the present invention (Series 1) are not yet optimized (at least, with respect to the optimal PSD).
4. The results of Series 1-3 (are also presented graphically in FIG. 10) can be explained as follows:

a. The large quantity of gas bubbles that are trapped around/within he Aragonite particles/crystals of the present invention reduce nₒ (or in other words: the large quantity of gas bubbles that are trapped around/within the Aragonite particles/crystals of the present invention increase the effective refractive index nₚ) in the above Lorentz-Lorentz equation. Thus, giving rise to H.P. values (Series 1) that can only be observed when using top quality commercial TiO₂ pigments (Series 2 and 3). The incorporation of air into the Aragonite powders that are obtained according to the present invention and into their products is corroborated many times by experimental resuls all along the description of this invention and

b. In addition, the huge number of very tiny crystals that can now be observed in the SEM pictures of the product of the present invention (FIGS. 11 and 12—done at an amplilification of ×100,000 to ×200,000, respectively) form the well known acicular (needle) shape Aragonite crystals (as they are presented in FIGS. 4, 6 and 8 at a magnification that does not exceed ˜×20,000). This new and surprising microstructure, which can not be observed in the SEM pictures (FIGS. 4, 6 and 8) and which is not present in e.g. OPACARB A40, the commercial product of SMI (FIGS. 13 and 14—done at an amplification of ×110,000 and ×200,000, respectively), can explain the enhanced dispersion of light that is being exhibited by the product of the present invention. This microstructure can enhance the dispersion of light though trapping of gas bubbles in the narrow indentations (e.g. holes and tunnels) and by forcing multiple events of light dispersion, provided that its producer is aware of these facts, is equipped with the proper methodology and simple tests that enable him to identify the product of the present invention (especially, the specific gravity of the product, as is described in EXAMPLES 14(A), 14(C) and 14(E)), and takes the proper care to handle this product in the down-stream operations (mainly, by avoiding the displacement the trapped gas bubbles in the tiny voids when high opacity and low specific gravity products are desired)

The fact that the product of the present invention is capable of competing quite effectively with the top quality commercial TiO₂ pigments in dispersing of light in e.g. coatings by combining the effect of trapped air bubbles in narrow voids and the effect of very large numbers of light dispersion events, which are caused by this unique microstructure of the product of the present invention, is unpreceded in the literature concerning CaCO₃ products. It is quite possible that in the near future these unexpected phenomena will lead to products of which effective refractive indices will even exceed that of TiO₂ (i.e. once the process of the present invention will be optimized with respect to active agents, processing conditions and optimal PSD).

5. EXAMPLE 19(A) can serve as a test to determine which CaCO₃ particles belong to the present invention. Namely, a coating that includes a single product of CaCO₃ at ˜55% (wt) and exhibiting a H.P. value that is not less than 90 will reflect the fact that it belongs to the present invention. This include mixtures of CaCO₃, as each test will be conducted using a definite product that contains only CaCO₃.

While the invention has been described with respect to a limited number of embodiments, it will be appreciated that many variations, modifications and other applications of the invention may be made.

1. A composition of matter comprising precipitated aragonite calcium carbonate having a specific gravity below about 2.5 g/cm³.

2. A composition of matter according to claim 1, wherein the specific gravity is less than about 2.3 g/cm³.

3. A composition of matter according to claim 1, wherein the specific gravity is less than about 2.1 g/cm³.

4. A composition of matter according to claim 1, wherein the specific gravity is less than about 2 g/cm³.

5. A composition of matter according to claim 1, wherein the specific gravity is less than about 1.8 g/cm³.

6. A composition of matter according to claim 1, wherein the specific gravity is less than about 1.5 g/cm³.

7. A composition of matter according to claim 1, wherein the particulate precipitated aragonite calcium carbonate of the invention, has a specific gravity below about 2.5 g/cm³, when determined under the following conditions:
(a) after drying for 12 hours at 120° C.; or (b) after drying for 12 hours at 120° C. and subsequently ignited for 8 hours at 500° C.

8. A composition of matter according to claim 1, comprising at least one carboxylic acid calcium salt, wherein the carboxylic acid is of the formula RCOOH, wherein R may be a saturated or unsaturated, optionally substituted aliphatic group, e.g. a hydrocarbon group, that contains 7-21 carbon atoms or carboxylate salt, ester, anhydride, acyl halide or ketene thereof

9. A composition of matter according to claim 1, comprising one or more carboxylic acid calcium salt, wherein the carboxylic acid is of the formula CₙH_{2n±1}COOH, wherein n is 8-17, or their carboxylate salt, ester, anhydride, acyl halide or ketene thereof.

10. A composition of matter according to claim 1, comprising one or more carboxylic acid calcium salt, wherein the carboxylic acid is of the formula CH₃(CH₂)ₙCOOH, wherein n is 7-16, or their carboxylate salts, esters, anhydrides, acyl halides or ketene thereof.

11. A composition of matter according to claim 1, having a hiding power of at least 90.

12. A process for producing a particulate precipitated aragonite calcium carbonate, which comprises reacting an aqueous calcium hydroxide slurry with a gas selected from the group consisting of carbon dioxide and a gas containing it and selecting the process parameters such that the resulting aragonite calcium carbonate has a specific gravity of less than about 2.5 g/cm³.

13. A process according to claim 12, wherein the process parameters comprising adding at least one active agent selected from carboxylic acids of formula RCOOH, wherein R may be a saturated or unsaturated, optionally substituted aliphatic group, e.g. a hydrocarbon group, that contains 7-21 carbon atoms or carboxylate salts, esters, anhydrides, acyl halides or ketenes thereof

14. A process according to claim 13, wherein said at least one active agent is selected from the group consisting of carboxylic acids of formula CₙH_{2n±1}COOH, wherein n is 8-17, or carboxylate salts, esters, anhydrides, acyl halides or ketenes thereof.

15. A process according to claim 14, wherein:
said at least one active agent is selected from the group consisting of carboxylic acids of formula CH₃(CH₂)ₙCOOH, where n is 7-16, their carboxylate salts, their acid anhydrides, their esters, their acyl halides and their ketenes of the formula CH₃(CH₂)ₙC═C═O, where n is 6-15;
said at least one active agent has a concentration within the range between about 0.2 wt. % and about 10 wt. %, calculated as carboxylic acid(s) and based on the weight of calcium carbonate;
said slurry contains calcium hydroxide in a concentration within the range of from about 3 to about 30 wt. %;
said slurry has a pH range of from about 8 to about 11; and
the reaction is carried out at a temperature in the range between about 60° C. and the boiling temperature of the reaction mixture.

16. A process according to claim 12, being a continuous or semi-continuous (intermittent) process.

17. A process according to claim 12, wherein the slurry is mixed with a mixer having a peripheral speed (tip-speed) above about 5 m/sec.

18. A process according to claim 12, wherein said at least one active agent is added in a manner selected from introduction into a carbonation reactor and premixing with said calcium hydroxide slurry prior to reaction with said gas.

19. A process according to claim 15 wherein
said at least one active agent is selected from the group consisting of carboxylic acids of formula CH₃(CH₂)ₙCOOH, where n is 7-12, and the calcium salts thereof;
said concentration of the at least one active agent is within the range between about 0.3 wt. % and about 5 wt. %, calculated as carboxylic acid(s) and based on the weight o calcium carbonate;
said slurry contains calcium hydroxide in a concentration within the range of from about 4 to about 20 wt. %;
said pH is within the range of from about 9 to about 10;
said temperature is in the range between about 80° C. and the boiling temperature of the reaction mixture;
said mode of operation is a continuous mode of operation.

20. A process according to claim 19, wherein:
said active agent is selected from the group consisting of nonanoic acid, decanoic acid, undecanoic acid dodecanoic acid and the calcium salts thereof;
said concentration of said at least one active agent is within the range between about 0.4 wt. % and about 3 wt. %, calculated as carboxylic acid and based on the weight of calcium carbonate;
said temperature is in the range between about 90° C. and the boiling temperature of the reaction mixture; and
said slurry contains calcium hydroxide in a concentration within the range of from 5 wt. % to 15 wt. %.

21. A process according to claim 20, wherein:
said active agent is selected from the group consisting of decanoic acid and the calcium salts thereof;
said concentration of said at least one active agent is within the range between about 0.4 wt. % and about 3 wt. %, calculated as carboxylic acid and based on the weight of calcium carbonate;
said temperature is in the range between about 90° C. and the boiling temperature of the reaction mixture; and
said slurry contains calcium hydroxide in a concentration within the range of from about 5 wt. % to about 15 wt. %.

22. A process according to claim 14, wherein:
said at least one active agent is selected from the group consisting of carboxylic acids of formula CₙH₂ₙ₋₁COOH, where n is 8-17, their carboxylate salts, their acid anhydrides, their esters, their acyl halides and their ketenes of the formula CₙH₂ₙ₋₁C═C═O, where n is 7-16;
said concentration of the at least one active agent is within the range between about 0.2 wt. % and about 10 wt. %, calculated as carboxylic acid(s) and based on the weight of calcium carbonate;
said slurry contains calcium hydroxide in a concentration within the range of from about 3 to about 30 wt. %;
said pH is within the range of from about 8 to about 11;
said temperature is in the range between about 60° C. and the boiling temperature of the reaction mixture;
said mode of operation is selected from a continuous and a semi-continuous (intermittent) mode of operation;
said mixer peripheral speed (tip-speed) is above 5 m/sec;
said at least one active agent is added in a manner selected from introduction into the carbonation reactor and premixing with said calcium hydroxide slurry prior to reaction with said gas.

23. A process according to claim 22, wherein:
said active agent is selected from the group consisting of undecylenic acid and the calcium salts thereof;
said concentration of said at least one active agent is within the range between about 0.4 wt. % and about 3 wt. %, calculated as carboxylic acid and based on the weight of calcium carbonate;
said temperature is in the range between about 80° C. and the boiling temperature of the reaction mixture;
said pH is within the range of from about 9 to about 10; and
said slurry contains calcium hydroxide in a concentration within the range of from about 5 to about 15 wt. %.

24. A process according to claim 12, which is conducted as a flotation process in a flotation cell.

25. A process according to claim 24, and substantially as hereinbefore described with reference to FIG. 3 of the attached drawings.

26. A particulate precipitated aragonite according to claim 9, which contains at least one calcium salt of carboxylic acids selected from those of the general formulae CₙH₂₊₁COOH and CₙH₂ₙ₋₁COOH, where n is 8-17, in an amount between 0.2 and 10 wt. %, calculated as carboxylic acid(s) and based on the weight of calcium carbonate.

27. A particulate precipitated aragonite according to claim 10, which contains at least one calcium salt of carboxylic acids selected from those of the general formula: CH₃(CH₂)ₙCOOH, where n=7-16, in an amount between about 0.2 and about 10 wt. %, calculated as carboxylic acid(s) and based on the weight of calcium carbonate.

28. A particulate precipitated aragonite according to claim 9, which contains at least one calcium salt of carboxylic acids selected from nonanoic acid, decanoic acid, undecanoic acid, dodecanoic acid, tetradecanoic acid, octadecanoic acid and undecylenic acid, in an amount between 0.2 and 10 wt. %, calculated as carboxylic acid(s) and based on the weight of calcium carbonate

29. A process according to claim 12, wherein each said specific gravity is determined substantially as described in Example 14(A).

30. A particulate precipitated aragonite according claim 1, wherein said specific gravity is determined substantially as described in Example 14(A).

31. A particulate precipitated aragonite according claim 7, wherein said specific gravity is determined substantially as described in Example 14(A) and 14(C).

32. A particulate precipitated aragonite according claim 11, wherein the hiding power is determined substantially as described in Example 19(A).

33. A substantially pure particulate precipitated aragonite according to claim 1, wherein the crystallographic purity (aragonite/(aragonite+calcite)) is at least 95%.

34. A particulate precipitated aragonite according to claim 1, wherein crystallographic purity (aragonite/(aragonite+calcite)) is at least 90%.

35. A particulate precipitated aragonite according to claim 1, wherein the crystallographic purity (aragonite/(aragonite+calcite)) is less than 90%.

36. A particulate precipitated aragonite according to claim 1, wherein the crystallographic purity (aragonite/(aragonite+calcite)) is more than 75%.

37. A particulate precipitated aragonite according to claim 1, wherein the crystallographic purity (aragonite/(aragonite+calcite)) is more than 50%.

38. A process for producing a particulate precipitated aragonite calcium carbonate, wherein said comprises reacting an aqueous calcium hydroxide slurry with a gas selected from the group consisting of carbon dioxide and a gas containing it, wherein the parameters of said process, including at least one active agent, mode of operation, operating concentrations of raw materials, operating temperature, operating mixer speed and operating pH, wherein said at least one active agent is selected from the group consisting of nonanoic acid, decanoic acid, undecanoic acid, dodecanoic acid, tetradecanoic acid, octadecanoic acid, and undecylenic acid, their carboxylate salts, their acid anhydrides, their esters, their acyl halides and their ketenes.

39. A process according to claim 38, wherein said process is further characterized by the following features:
said concentration of the at least one active agent is within the range between about 0.2 wt. % and about 10 wt. %, calculated as carboxylic acid(s) and based on the weight of calcium carbonate;
said slurry contains calcium hydroxide in a concentration within the range of from 3 to 30 wt. %;
said pH is within the range of from 8 to 11;
said temperature is in the range between 60° C. and the boiling temperature of the reaction mixture;
said mode of operation is selected from a continuous and a semi-continuous (intermittent) mode of operation;
said mixer peripheral speed (tip-speed) is above 5 m/sec.;
said at least one active agent is added in a manner selected from introduction into the carbonation reactor and premixing with said calcium hydroxide slurry prior to reaction with said gas.

40. A process according to claim 39, wherein said process is further characterized by at least one of the following features:
said at least one active agent is selected from the group consisting of nonanoic acid, decanoic acid, undecanoic acid, dodecanoic acid, tetradecanoic acid, octadecanoicacid, and undecylenic acid, and the calcium salts thereof;
said concentration of the at least one active agent is within the range between 0.3 wt. % and 5 wt. %, calculated as carboxylic acid(s) and based on the weight of calcium carbonate;
said slurry contains calcium hydroxide in a concentration within the range of from 4 to 20 wt. %;
said pH is within the range of from 9 to 10;
said temperature is in the range between 80° C. and the boiling temperature of the reaction mixture;
said mode of operation is a continuous mode of operation.

41. A process according to claim 40, wherein said process is further characterized by at least one of the following features, namely:
said active agent is selected from the group consisting of nonanoic acid, decanoic acid, undecanoic acid, undecylenic acid and the calcium salts thereof;
said concentration of said at least one active agent is within the range between about 0.4 wt. % and about 3 wt. %, calculated as carboxylic acid and based on the weight of calcium carbonate;
said temperature is in the range between about 90° C. and the boiling temperature of the reaction mixture; and
said slurry contains calcium hydroxide in a concentration within the range of from about 5 to about 15 wt. %.

42. A process according to claim 41, wherein said process is further characterized by at least one of the following features, namely:
(a) said active agent is selected from the group consisting of decanoic acid, undecylenic acid, and the calcium salts thereof,
(b) said concentration of said at least one active agent is within the range between 0.4 wt. % and 3 wt. %, calculated as carboxylic acid and based on the weight of calcium carbonate,
(c) said temperature is in the range between 90° C. and the boiling temperature of the reaction mixture, and
(d) said slurry contains calcium hydroxide in a concentration within the range of from 5 to 15 wt. %.

43. A process according to claim 38, wherein said process is conducted as a flotation process in a flotation cell.

44. A process according to claim 43, and substantially as hereinbefore described with reference to FIG. 3 of the attached drawings.

45. A particulate precipitated aragonite produced by the process of claim 38.

46. A substantially pure particulate precipitated aragonite according to claim 45, wherein the crystallographic purity (aragonite/(aragonite+calcite)) is at least 95%.

47. A particulate precipitated aragonite according to claim 45, wherein the crystallographic purity (aragonite/(aragonite+calcite)) is at least 90%.

48. A particulate precipitated aragonite according to claim 45, wherein the crystallographic purity (aragonite/(aragonite+calcite)) is more than 75%.

49. A particulate precipitated aragonite according to claim 45, wherein the crystallographic purity (aragonite/(aragonite+calcite)) is more than 50%.

50. A particulate precipitated aragonite according to claim 45, wherein the crystallographic purity (aragonite/(aragonite+calcite)) is more than 40%.

51. A particulate precipitated aragonite according to claim 1, of which SEM picture is substantially similar to those in FIGS. 11 and 12.

52. A particulate precipitated aragonite obtained by the process of claim 12, wherein the SEM picture is substantially similar to those in FIGS. 11 and 12.

53. A particulate precipitated aragonite of claim 45, wherein the SEM picture is substantially similar to those in FIGS. 11 and 12.

54. A particulate precipitated aragonite wherein the SEM picture of said particulate is substantially similar to those in FIGS. 11 and 12.

55. A composition which comprises a particulate precipitated aragonite as defined in any one of claims 1 to 11, 26 to 28 or 45 to 54, wherein said composition being selected from a coating composition, a paper composition, a plastics composition, a rubber composition, an adsorbent composition, a powder detergent composittion, a pharmaceutical composition, an agrochemical composition, a flavor composition, a fragrance composition, a food composition, a feed composition, a sunscreen composition or a conductive powder composition.

56. A composition according to claim 55, wherein said composition comprises substantially dry particulate precipitated aragonite.

57. A composition according to claim 55, wherein said composition is selected from a coating composition, a paper composition, a pharmaceutical composition, an agrochemical composition, a flavor composition, a fragrance composition, a food composition, a feed composition or a sunscreen composition, and which comprises particulate precipitated aragonite in aqueous dispersion.
